(19) United States
(12) Patent Application Publication
(10) Pub. No.: US 2022/0304307 A1
(43) Pub. Date: Sep. 29, 2022

(54) SYNERGISTIC MIXTURES FOR ARTHROPOD TOXICITY AND REPELLENCY

(71) Applicant: University of Florida Research Foundation, Incorporated, Gainesville, FL (US)

(72) Inventors: Liu Yang, Gainesville, FL (US); Gary Richoux, Gainesville, FL (US); Edmund J. Norris, Gainesville, FL (US); Jeffrey R. Bloomquist, Gainesville, FL (US)

(21) Appl. No.: 17/630,843

(22) PCT Filed: Jul. 30, 2020
(86) PCT No.: PCT/US2020/044292

§ 371 Date: Jan. 27, 2022

Related U.S. Application Data
(60) Provisional application No. 62/880,503, filed on Jul. 30, 2019.

(51) Int. Cl. A01N 53/00; C07C 69/743

(52) U.S. Cl. CPC A01N 53/00; C07C 69/743; C07C 2601/02; C07C 2601/04; C07C 2601/08; C07C 2601/14

(57) ABSTRACT

The subject matter described herein is directed to compounds, synergistic compositions, and methods for repelling arthropods. The compositions comprise acids, esters, aldehydes, or alcohols of pyrethroid-type compounds. The compounds, particularly the pyrethroid-type acids, demonstrate excellent repellency when applied both alone and in synergistic compositions.

### CROSS-REFERENCE TO RELATED APPLICATIONS

This application claims the benefit of priority to U.S. Provisional Patent Application No. 62/880,503, filed on Jul. 30, 2019, the content of which is incorporated by reference herein in its entirety for all purposes.

### GOVERNMENT INTEREST

This invention was made with government support under 58-0208-5-01 & 59-6036-8-001 awarded by The United States Department of Agriculture, Agriculture Research Services and under U01 CK000510 awarded by The United States Center for Disease Control. The government has certain rights in the invention.

### FIELD

The subject matter described herein is directed to arthropod repellent compounds and synergistic compositions, particularly those based on acids of pyrethroids.

### BACKGROUND

Biting insects are a nuisance and are also known to be carriers of disease. Compositions that repel insects, particularly insect pests such as mosquitoes, flies and fleas, are widely used to prevent animals from being bitten by such insects. The importance of preventing biting is essentially two-fold. Firstly, there are a number of insects which are capable of infecting animals with disease causing parasites, an example being the transmission of Plasmodium falciparum by mosquitoes to cause malaria. Secondly, in many cases whether disease is transmitted or not, the bite can be extremely irritating.

Insect repellents that act to repel insects from surfaces, including the skin of animals, are commercially available. They are designed to reduce or prevent the tendency of the insect to contact the surface, thereby preventing the biting. Over the years, a number of different oils, greases, ointments, sprays, and powders have been employed as insect repellents with varying degrees of success. Other delivery methods, such as fans, heated emanators, and burning coils, have also been developed with the purpose of repelling insects. Oil of citronella was reported to be an effective insect repellent as early as 1901. Another natural product, nicotine from tobacco, was used as a repellent as long ago as 1760. Since World War II, a number of synthetic insect repellents have been introduced.

One such repellent is N,N-diethyl-m-toluamide (DEET). It is a synthetic insect repellent that has demonstrated superior insect repellency in comparison to other repellents, including natural insect repellents. However, there is concern that repeated DEET exposure may have harmful consequences. For example, possible DEET side effects are irritability, confusion, insomnia, and even seizures. For these reasons, caution is generally recommended with regard to the use of DEET as an insect repellent. This is especially true when DEET is applied to children, who are more susceptible to the potentially injurious effects of exposure to DEET.

Pyrethrins, which are cyclopropanecarboxylates, the naturally occurring extracts from the blossoms of pyrethrum flowers (Chrysanthemum cinerariae-folium) grown mainly in East Africa, were widely used as insecticides before the advent of synthetic materials such as DDT. The pyrethrins are effective in killing a wide range of insect species, and they also function as insect repellents [Burden, Pest Control, 43, 16 (1975)]. Although they display relatively low toxicity toward mammals and do not leave harmful residues, they undergo rapid biodegradation, they have poor photooxidative stability, their availability is uncertain, and it is costly to extract and process them.

Efforts have been underway to produce synthetic cyclopropanecarboxylate insecticides, pyrethroids, which would overcome these disadvantages. Pyrethroids are known to have significant insecticidal effects and are effective in repelling and/or killing blood sucking insects. 5-Benzyl-3-furylmethyl chrysanthemate (resmethrin) [Elliott, et al., Nature, 213, 493 (1967)], a powerful contact insecticide, was an early success. A notable result of these efforts was the discovery of the pyrethroid, 3-phenoxybenzyl 3-(β,β-dichlorovinyl)-2,2-dimethylcyclopropanecarboxylate (permethrin), having the combination of toxicity and knockdown attributes of the natural pyrethrins and resmethrin together with a previously unattained level of photooxidative stability [Elliott, et al., Nature, 246, 169 (1973); S. African Patent 73/3528]. Subsequently, permethrin was determined to be the best insect repellent for clothing and field treatment of U.S. military combat uniforms, and has been used as the standard repellent treatment of uniforms since 1991 (Armed Forces Pest Management Board Technical Guide No. 36, Personal Protective Measures against Insects and other Arthropods of Military Significance).

Of course, the repellent treatment will need to be re-applied. For economic and safety reasons, it would be beneficial to be able to use the least amount of active agents that provide the desired efficacy. The same is true for spatial repellents, vapor-active and deployed to keep a room, house, or tent, free of biting insects. Therefore, what is needed are highly efficacious compounds, especially those that can synergize with other compounds, whereby the concentrations and amounts of compounds required for efficacy is lessened. The subject matter described herein addresses the shortcomings of the art.

### BRIEF SUMMARY

In certain embodiments, the subject matter described herein is directed to a compound of Formula I

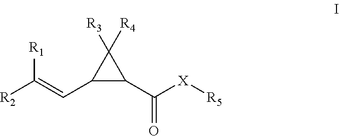

wherein R₁ and R₂ are each independently selected from the group consisting of hydrogen, halo, haloalkyl, haloaryl, C₆-C₁₂ heteroaryl, C₆-C₁₂ aryl, alkenyl, alkynyl, and C₁-C₃ alkyl; or

wherein R₁ and R₂ are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₃ and R₄ are each independently selected from hydrogen, haloalkyl, alkenyl, alkynyl, and C₁-C₆ alkyl;

R₅ is selected from the group consisting of linear or branched C₅-C₁₂ alkyl, haloalkyl, alkenyl, alkynyl, C₃-C₅ cycloalkyl, C₃-C₁₂ cycloalkylalkyl, C₆-C₁₂ heteroaryl, and C₃-C₁₂ heterocycloalkyl; and

X is selected from the group consisting of O, NH, and S.

In certain embodiments, the subject matter described herein is directed to a composition comprising a compound of Formula II′ or a compound of Formula III′:

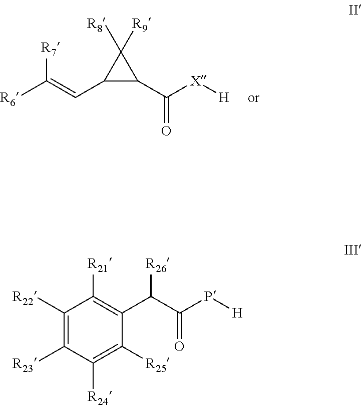

where R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl, and

a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1,

wherein the composition is synergistic, in admixture with a carrier.

In certain embodiments, the subject matter described herein is directed to a composition comprising a compound of Formula IV, a compound of Formula V, or a compound of Formula VI:

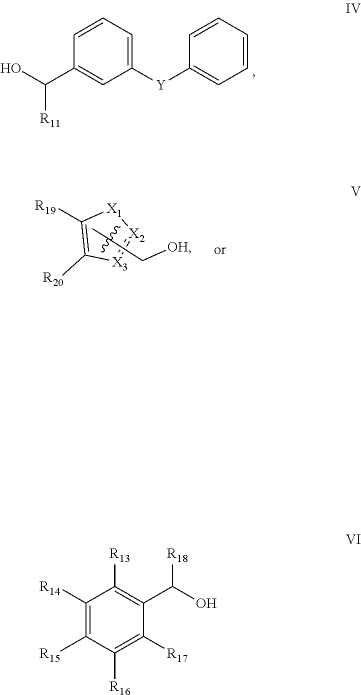

wherein Y is selected from the group consisting of O, NH, and S;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, haloalkyl, alkenyl, alkynyl, C₁-C₆ alkyl, and C₁-C₃ alkyl-alkoxy;

R₁₈ is selected from the group consisting of hydrogen, haloalkyl, alkenyl, alkynyl, and linear or branched C₁-C₆ alkyl; and

a compound of Formula II′ or a compound of Formula III′:

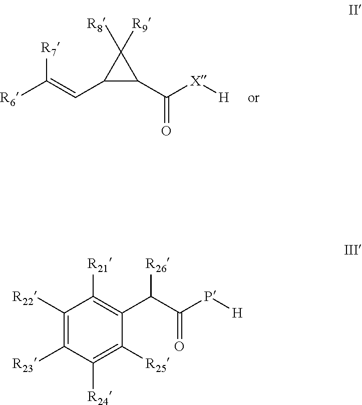

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl,

wherein the composition is synergistic, in admixture with a carrier.

In certain embodiments, the subject matter described herein is directed to a composition comprising a compound of Formula VII

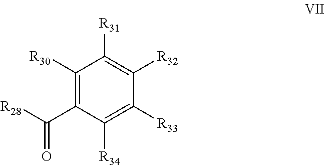

wherein R₂₈ is selected from the group consisting of hydrogen, haloalkyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

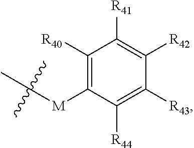

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, alkoxy, halo, haloalkyl, alkenyl, alkynyl, cyano, hydroxy, and

a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, VUAA-1, nootkatone, a compound of Formula II′, a compound of Formula III′, a compound of Formula IV, a compound of Formula V, and a compound of Formula VI, wherein the composition is synergistic, in intimate admixture with a carrier.

In certain embodiments, the subject matter described herein is directed to a composition comprising a compound of Formula I′ and a compound of Formula II′ or a compound of Formula III′, wherein the composition is synergistic, in admixture with a carrier.

In certain embodiments, the subject matter disclosed herein is directed to a method of repelling an arthropod from an object or locus, comprising contacting said object or locus with a composition comprising a compound of Formula II′ or a compound of Formula III′ and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, nootkatone, diethyl carbate, geraniol, citronellol, citronellal, citral, oil of lemon eucalyptus, cinnamaldehyde, and VUAA-1, wherein the composition is synergistic, in admixture with a carrier.

In certain embodiments, the subject matter disclosed herein is directed to a method of repelling an arthropod from an object or locus, comprising contacting said object or locus with a composition comprising a compound of Formula IV, a compound of Formula V, or a compound of Formula VI and a compound of Formula II′ or a compound of Formula III′, wherein the composition is synergistic, in admixture with a carrier.

In certain embodiments, the subject matter disclosed herein is directed to a method of repelling an arthropod from an object or locus, comprising contacting said object or locus with a composition comprising a compound of Formula VII and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, nootkatone, VUAA-1 a compound of Formula II′, a compound of Formula III′, a compound of Formula IV, a compound of Formula V, and a compound of Formula VI, wherein the composition is synergistic, in admixture with a carrier.

In certain embodiments, the subject matter disclosed herein is directed to a method of repelling an arthropod from an object or locus, comprising contacting said object or locus with a composition comprising a compound of Formula I′ and a compound of Formula II′ or a compound of Formula III′, wherein the composition is synergistic, in admixture with a carrier.

In certain embodiments, the subject matter disclosed herein is directed to a method for controlling one or more arthropods, comprising contacting the one or more arthropods with a synergistic composition comprising a compound of Formula II′ or a compound of Formula III′ and a pyrethroid, wherein the synergistic composition produces, when the one or more arthropods are brought into contact with the synergistic composition, a combined toxicant effect greater than the sum of the separate toxicant effects from the compound of Formula II′ or Formula III′ and the pyrethroid, at comparable concentrations.

In certain embodiments, the subject matter disclosed herein is directed to a method for controlling one or more arthropods, comprising exposing said one or more arthropods to a synergistic composition comprising a compound of Formula II′ or a compound of Formula III′ and a pyrethroid, wherein the synergistic composition produces, when said one or more arthropods are exposed to the synergistic composition, a combined toxicant effect greater than the sum of the separate toxicant effects from the compound of Formula II′ or Formula III′ and the pyrethroid, at comparable concentrations.

In certain embodiments, the subject matter disclosed herein is directed to a method of repelling an arthropod from an object or locus, comprising contacting said object or locus with a composition comprising a carrier and a compound of Formula II, Formula III, Formula IV, Formula V, Formula VI, or Formula VII

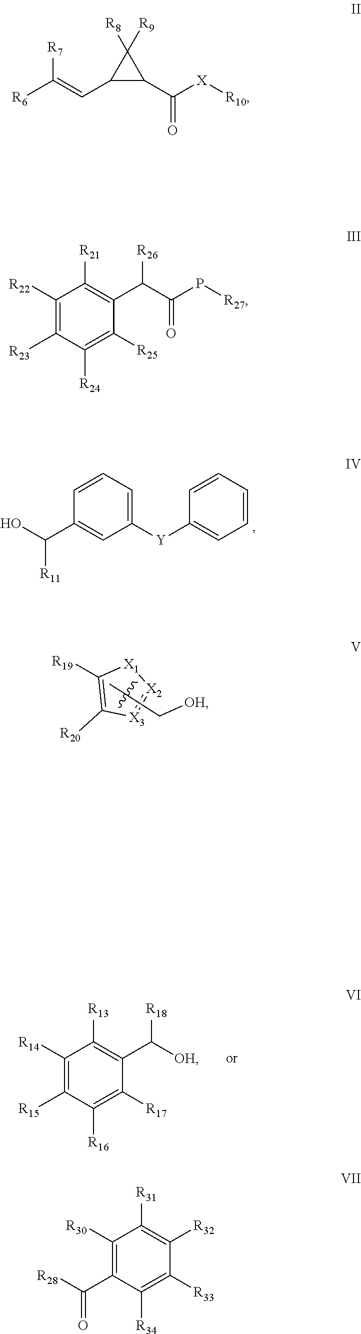

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R₆ and R₇ are taken together with the carbon to which they are attached to form a C₆-C₁₂ heteroaryl, C₆-C₁₂ aryl, C₃-C₁₂ cycloalkyl, haloaryl, or C₃-C₂ heterocycloalkyl;

R₈ and R₉ are each independently selected from hydrogen haloalkyl, alkenyl, alkynyl, and C₁-C₆ alkyl;

R₁₀ is selected from the group consisting of hydrogen, haloalkyl, alkenyl, alkynyl, linear or branched C₁-C₁₂ alkyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, haloalkyl, alkenyl, alkynyl, alkoxy, halo, C₁-C₆ alkyl, and C₁-C₃ alkyl-alkoxy;

P is selected from the group consisting of O, NH, and S;

R₂₆ is selected from the group consisting of hydrogen, haloalkyl, haloalkyl, alkenyl, alkynyl, and linear or branched C₁-C₆ alkyl;

R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, haloalkyl, alkenyl, alkynyl, C₁-C₆ alkyl, CN, halo, and haloalkyl;

R₁₉ and R₂₀ are each independently selected from the group consisting of hydrogen, C₁-C₆ alkyl, cycloalkylalkyl, arylalkyl, heterocycloalkyl, heteroarylalkyl, cycloalkyl, halo, haloalkyl, alkenyl, alkynyl, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, alkoxy, halo, haloalkyl, alkenyl, alkynyl, cyano, hydroxy, and

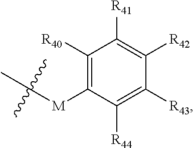

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy.

In certain embodiments, the subject matter described herein is directed to a composition comprising an essential oil and a compound selected from the group consisting of a pyrethroid, a compound of Formula II, Formula III, Formula IV, Formula V, Formula VI, and Formula VII, wherein the composition is synergistic, in admixture with a carrier.

In certain embodiments, the subject matter described herein is directed to a method of repelling an arthropod from an object or locus, comprising contacting said object or locus with a composition comprising an essential oil, and a second compound selected from the group consisting of a pyrethroid, a compound of Formula II, Formula III, Formula IV, Formula V, Formula VI, and Formula VII, wherein the composition is synergistic, in admixture with a carrier.

Other embodiments are also described.

### BRIEF DESCRIPTION OF THE DRAWINGS

FIG. 1 shows an example of DEET and control responses in a glass tube assay. Mosquitoes moved away from the DEET treated side (top right), while remaining evenly distributed on either side of the midline for acetone controls (bottom). The EC₅₀ for DEET in the assay was 39 μg/cm². The tubes as shown were held horizontally. With certain compounds, the tubes were held vertically, during which they provided a more consistent response. Without wishing to be bound by theory, it is understood that these response-orientation results are due to the compounds' different chemical diffusion properties.

FIG. 2 shows the repellency proportion of intact commercial pyrethroids and pyrethrum after 1 hr of exposure at 100 μg/cm² measured as a proportion of flies on the chemically-treated side, where a score of 0.5=no repellency and 0=full repellency. Bars are mean±SEM. Asterisks indicate a significant difference between tested chemicals and acetone control in a T-test (***P<0.001, ****P<0.0001).

FIG. 3 shows the repellency proportion of several pyrethroid acids after 1 hr of exposure at 100 μg/cm² measured as a proportion of flies on the chemically-treated side, where a score of 0.5=no repellency and 0=full repellency. Bars are mean±SEM. Asterisks indicate a significant difference between tested chemicals and acetone control in a T-test (***P<0.001, ****P<0.0001).

FIG. 4 shows a plot of several pyrethroid and pyrethrin-based acid concentration-response curves for Ae. aegypti female repellency in glass tubes. Symbols are the mean with SEM bars omitted for clarity. The dashed line represents the 50% level of repellency. MFA=metofluthrin acid, BFA=bifenthrin acid, PhenA=phenothrin acid, TCA=1-R-trans-chrysanthemic acid, TFA=transfluthrin acid, DMA=deltamethrin acid, FTFA=FTFA, and FVA=fenvalerate acid.

FIG. 5 shows how in mixtures, TFA (50 μg/cm²), which is inactive by itself, synergizes some commercial and experimental (VUAA-1) repellents, as well as a contact irritant (permethrin). The dashed horizontal line indicates the 20% repellency level and numbers in parentheses for each compound indicate said treatment in μg/cm². Numbers are absent for permethrin and VUAA-1 because repellency was not observed at the highest concentration tested (1-2 mg/cm²). Bars are means 1 SEM. Asterisks indicate significant difference between tested chemicals alone, compared to the responses observed in combination with TFA (*P<0.05, **P<0.01, ***P<0.001, ****P<0.0001).

FIG. 6 shows an experiment with TFA and TCA demonstrating a leftward shift to lower concentrations in the response curves for 2-undecanone.

FIG. 7A shows the repellency observed when DEET (18 μg/cm²) is applied side-by-side (S) or mixed (M) with TFA (50 μg/cm²). At these two fixed concentrations, a synergistic effect was observed when applied side-by-side.

FIG. 7B shows the repellency observed when DEET (18 μg/cm²) is applied side-by-side (S) or mixed (M) with TFA (50 μg/cm²). Shown are DEET concentration-response curves±TFA under both filter treatment regimes. Symbols are means±SEM, with curves fit as described in Example 2.

FIG. 8 shows the repellency of transfluthrin (TF) alone and when mixed with TFA. Symbols are means±SEM. The EC₅₀ for transfluthrin alone was 0.5 μg/cm² and 0.11 when mixed with TFA. Thus, the synergism ratio is about 5.

FIG. 9A shows the synergistic repellency interactions of TFA with citronella, when the latter is applied at a nominal EC₂₀, the concentration that gives 20% repellency. Symbols are means±SEM.

FIG. 9B shows the absence of synergistic interactions of transfluthrin (TF) and citronella. Symbols are means 1 SEM.

FIG. 10A shows EAG responses (blank substracted) of compounds (10 mg) (TF, TFA, and TCA) compared to the no odorant blank (CTL). Mean EAG amplitudes of compounds (10 mg) are shown as bars±SEM and ***P<0.001 is from t-test comparisons of matched responses for each compound (chemical vs. control and OR vs. PR).

FIG. 10B shows EAG responses of OR (susceptible) and PR (resistant) strains of Ae. aegypti.

FIG. 11A shows repellency of pyrethroid alcohols screened at 100 μg/cm². Bars are means±SEM. (**P<0.01; ***P<0.001). Asterisks indicate significant difference (*P<0.05) at the indicated concentrations of TF-OH.

FIG. 11B shows transfluthrin alcohol (TF-OH) synergized by 50 μg/cm² TFA. Symbols are means±SEM.

FIG. 12A shows mixture studies with GMR 134 in combination with TFA (50 μg/cm²) and TCA (10 μg/cm²). Symbols are means 1 SEM. GMR 134 had an EC₅₀=6.5 μg/cm², +TFA=0.4, +TCA=0.7.

FIG. 12B shows mixture studies with GMR 134 in combination with citronella (20 μg/cm²). Symbols are means 1 SEM. GMR 134 had an EC₅₀=6.5 μg/cm², and +citronella=0.41, giving a synergism ratio of 16.

FIG. 13 illustrates the evaluation of synergistic repellency of pyrethroid alcohol mixtures with GMR 134, all screened alone at 100 μg/cm² and when mixed with GMR 134 at 2 μg/cm². Bars are means±SEM. Vertical bars denote paired comparisons of the different alcohols alone and with GMR 134.

FIG. 14 shows that pyrethroid-type acids, such as TFA and TCA, can synergize one another. Symbols are means±SEM. TCA EC₅₀=20 μg/cm² and +TFA=1.3 μg/cm², giving a synergism ratio=15.

FIG. 15 shows an illustrative concentration-response curve for GMR 125 against Ae. aegypti females, along with a generic structure for TFA esters and the structure of GMR 125.

FIG. 16 shows how the pyrethroid acid TFA can synergize its cyclopropyl ester (GMR126). Symbols are means 1 SEM. GMR 126 EC₅₀=8.5 μg/cm² and +TFA=0.35 μg/cm², giving a synergism ratio=24.

FIG. 17A shows the strong synergistic effect of citronella on the concentration-dependence of VUAA-1, where VUAA-1 is applied alone (open squares) and under co-exposure with citronella (closed circles). Symbols are means±SEM.

FIG. 17B shows the differential repellency effects of citronella (no effect) and TFA (synergism) with DEET, applied alone (grey bars=means±SEM), and in combination applied side by side (white bars=means±SEM).

FIG. 18 shows screening of TFA esters and certain pyrethroid acids for synergism with citronella. Bars are means±SEM, where white bars represent compound alone and grey bars with added citronella. The dashed line is the response to 20 μg/cm² citronella in these experiments and the number in parentheses is the EC₂₀ of the tested ester in g/cm². Asterisks indicate statistical significance (*P<0.05, **P<0.01, ***P<0.001).

FIG. 19 shows the screening of TFA esters for possible synergism with citronella. Bars are means±SEM; white and grey bars as defined previously. The dashed line is the response to g/cm² citronella in these experiments and the number in parentheses is the EC₂₀ of the tested ester in g/cm². The only strong positive response was with TFA (***P<0.001).

FIG. 20A shows how TFA (50 μg/cm²) synergizes the 24 h toxicity of transfluthrin. Symbols are means±SEM.

FIG. 20B shows how citronella synergizes the toxicity of metofluthrin. Symbols are means±SEM.

FIG. 21 shows concentration-response curves for repellent mixtures of GMR 134 with and without TFA (50 μg/cm²) against the insecticide-susceptible Orlando (OR) and pyrethroid-resistant Puerto Rico (PR) strains of Ae. aegypti. Symbols are means t SEM. Note that the resistance in the Puerto rico strain has little effect on this synergistic mixture.

FIG. 22 shows un-substituted benzaldehyde repellency on the Orlando strain of Ae. aegypti mosquitoes and its synergism by TFA. Symbols are means 1 SEM.

FIG. 23 shows concentration-response curves for repellency to Ae. aegypti females by GMR 126 alone and with TFA (50 μg/cm²) applied as a mixture (M) or separate (S) treated halves of the same filter. Symbols are mean±SEM. Synergism is reduced, but not eliminated by separating the treatments.

FIG. 24 shows a graph displaying the mortality of plant essential oils applied at various concentrations alone or in combination with 0.1 μg/cm² metrofluthrin in vapor toxicity assays on insecticide-susceptible Aedes aegypti (Orlando strain) females. Co-toxicity factors (CF) are reported as a measure of synergism, as described by Mansour et al. 1966, Toxicological studies on the Egyptian cotton Leafworm, Prodenia litura. VI. Potentiation and antagonism of organophosphorus and carbamate insecticides. J. Econ Entomol 59(2): 307-311. CF values greater than 20 are considered synergistic.

FIG. 25 shows a graph displaying the mortality of plant essential oils applied at various concentrations alone or in combination with 0.1 μg/cm² metrofluthrin in vapor toxicity assays on insecticide-susceptible Anopheles gambiae (G3 strain) females. Synergism interpreted as described in the legend to FIG. 24.

FIG. 26 shows a plot displaying the synergistic effect of metrofluthrin in combination with citronella oil at its EC₂₀ for repellency and at the higher concentration of 100 μg/cm² against the susceptible Orlando strain of strains of Ae. aegypti.

FIG. 27 shows a plot displaying the synergistic effect of metrofluthrin in combination with citronella oil against the pyrethroid-resistant Puerto Rico strain of Ae. aegypti.

FIG. 28 shows a plot displaying the synergistic effect of empenthrin in combination with citronella oil against the pyrethroid-resistant Puerto Rico strain Aedes aegypti.

### DETAILED DESCRIPTION

Described herein are compounds, compositions, and methods that provide desired insect repellency. The compositions comprise acids, esters, aldehydes, amides, oximes, sulfamides, sulfonamides, or alcohols of pyrethroid-type compounds. The compounds demonstrate excellent repellency both alone and in synergistic compositions with other compounds. In certain embodiments, the pyrethroid-type compounds can also synergize the toxicity of pyrethroids, thereby more effectively controlling insects.

Pyrethroids typically contain ester linkages (Table 1-1). Carboxylesterase hydrolysis of pyrethroids is considered a detoxication reaction that will produce a pyrethroid acid and alcohol. Although pyrethroids such as transfluthrin and metofluthrin are often utilized in commercial repellents, it was generally accepted that the acids and alcohols of these pyrethroids were devoid of any biological activity, including repellency or toxicity. Without wishing to be bound by theory, it is understood that resistant strains of insects have up-regulated esterases as a mechanism of resistance, although binding to the esterase regardless of whether any hydrolysis occurs may be a contributing factor (K Wang, Y Huang, X Li, and M Chen, Functional Analysis of a Carboxylesterase Gene Associated With Isoprocarb and Cyhalothrin Resistance in Rhopalosiphum padi (L.). Frontiers in Physiology 9:992, 2018). As described herein, however, it has been found that pyrethroid alcohol and acids unexpectedly have repellent bioactivity. As such, the subject matter described herein is directed to methods of repelling insects using compositions comprising acids, alcohols, aldehydes, or esters of pyrethroid compounds. In certain embodiments, the repellent bioactivity of the pyrethroid-type acid, alcohol, aldehyde, or ester is greater than that of the parent pyrethroid compound. Furthermore, the methods and compositions disclosed herein are effective, even at low concentrations.

TABLE 1-1
Common Pyrethroids

| Pyrethroid | Structure |
|---|---|
| Pyrethrin I from natural pyrethrum | 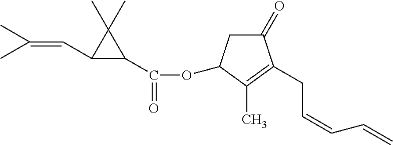 [SMILES: C=C/C=C\CC1=C(C)C(OC(=O)C2C(C=C(C)C)C2(C)C)CC1=O] |
| Tetramethrin | 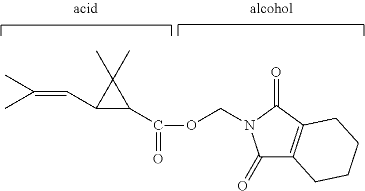 [SMILES: CC(C)=CC1C(C(=O)OCN2C(=O)C3=C(CCCC3)C2=O)C1(C)C] |
| Transfluthrin | 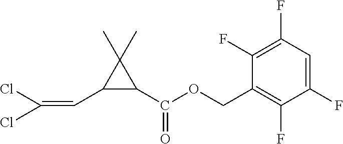 [SMILES: CC1(C)C(C=C(Cl)Cl)C1C(=O)OCc1c(F)c(F)cc(F)c1F] |
| Metofluthrin | 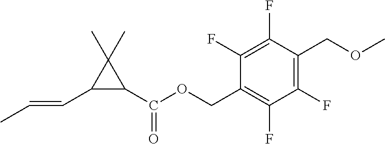 [SMILES: C/C=C/C1C(C(=O)OCc2c(F)c(F)c(COC)c(F)c2F)C1(C)C] |
| Bifenthrin | 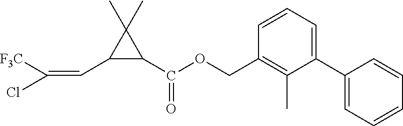 [SMILES: Cc1c(COC(=O)C2C(/C=C(/Cl)C(F)(F)F)C2(C)C)cccc1-c1ccccc1] |
| Kadethrin | 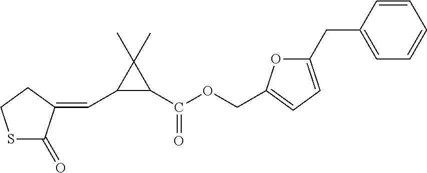 [SMILES: CC1(C)C(/C=C2\CCSC2=O)C1C(=O)OCc1ccc(Cc2ccccc2)o1] |
| Phenothrin | 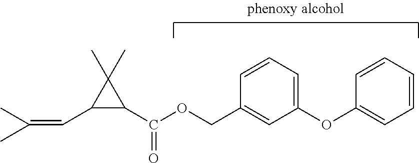 [SMILES: CC(C)=CC1C(C(=O)OCc2cccc(Oc3ccccc3)c2)C1(C)C] |
| Deltamethrin | 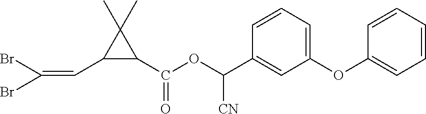 [SMILES: CC1(C)C(C=C(Br)Br)C1C(=O)OC(C#N)c1cccc(Oc2ccccc2)c1] α-cyano-3-phenoxybenzyl alcohol |
| Fenvalerate | 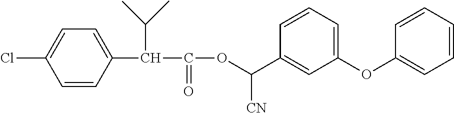 [SMILES: CC(C)C(C(=O)OC(C#N)c1cccc(Oc2ccccc2)c1)c1ccc(Cl)cc1] α-cyano-3-phenoxybenzyl alcohol |

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±50%. In certain other embodiments, the term “about” includes the indicated amount ±20%. In certain other embodiments, the term “about” includes the indicated amount ±10%. In other embodiments, the term “about” includes the indicated amount ±5%. In certain other embodiments, the term “about” includes the indicated amount 1%. In certain other embodiments, the term “about” includes the indicated amount ±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), 5 to 12 carbon atoms (i.e., C₅-C₁₂), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Arylhalo” refers to an aryl that is substituted with one or more halo substituents. In certain embodiments, the arylhalo may be substituted with 1, 2, 3, 4, 5, or 6 halo substituents.

“Arylalkyl” or “Aralkyl” refers to the group “aryl-alkyl-”, such as benzyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Alkyl-alkoxy” refers to the group “-alkyl-alkoxy.”

“Cycloalkylalkyl” refers to the group “cycloalkyl-alkyl-”.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen.

“Hydroxyalkyl” refers to an alkyl group as defined above, wherein one or more (e.g., 1 to 6, or 1 to 3) hydrogen atoms are replaced by a hydroxy group.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocycle ring contains 4- or 6-ring atoms, it is also referred to herein as a 4- or 6-membered heterocycle. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-.”

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfonyl are methylsulfonyl, ethylsulfonyl, phenylsulfonyl and toluenesulfonyl.

The compounds of the invention, or their agrochemically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic, optically pure, and enantiomerically/diastereomerically enriched forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

As used herein, “OR” refers to the Orlando insecticide-susceptible strain of Ae. aegypti.

As used herein, “PR” refers to the pyrethroid-resistant Puerto Rico (PR) strain of Ae. aegypti.

As used herein, “pyrethrum,” or “natural pyrethrins” refers to a botanical mixture of naturally derived insecticides commonly used in insect control. It is a mixture of six closely related chemical forms extracted from the Chrysanthemum plant (Pyrethrin I, Cinerin I, Jasmolin I, Pyrethrin II, Cinerin II, and Jasmolin II).

Additional definitions may also be provided below as appropriate.

### II. Compounds

In certain embodiments, the subject matter described herein is directed to compounds of Formula I:

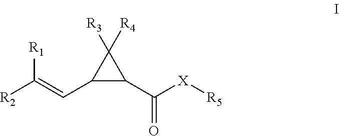

wherein R₁ and R₂ are each independently selected from the group consisting of hydrogen, halo, haloalkyl, C₆-C₁₂ heteroaryl, C₆-C₁₂ aryl, haloaryl, alkenyl, alkynyl, and C₁-C₃ alkyl; or

wherein R₁ and R₂ are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₃ and R₄ are each independently selected from hydrogen, haloalkyl, alkenyl, alkynyl, and C₁-C₆ alkyl;

R₅ is selected from the group consisting of linear or branched C₅-C₁₂ alkyl, haloalkyl, alkenyl, alkynyl, C₃-C₅ cycloalkyl, C₃-C₁₂ cycloalkylalkyl, C₆-C₁₂ heteroaryl, and C₃-C₁₂ heterocycloalkyl; and

X is selected from the group consisting of O, NH, and S.

In certain embodiments, R₃ and R₄ are each C₁-C₃ alkyl.

In certain embodiments, R₃ and R₄ are each methyl.

In certain embodiments, R₁ and R₂ are each halo.

In certain embodiments, R₁ and R₂ are each independently selected from chloro, bromo, and fluoro.

In certain embodiments, R₁ and R₂ are each independently chloro.

In certain embodiments, X is O.

In certain embodiments, X in NH.

In certain embodiments, R₅ is selected from the group consisting of linear C₅-C₁₂ alkyl, haloalkyl, and C₃-C₅ cycloalkyl.

In certain embodiments, R₅ is selected from the group consisting of pentyl, trifluoroethyl, cyclobutyl, cyclopentyl, and cyclopropyl.

In certain embodiments, the compound of Formula I is selected from the group consisting of:

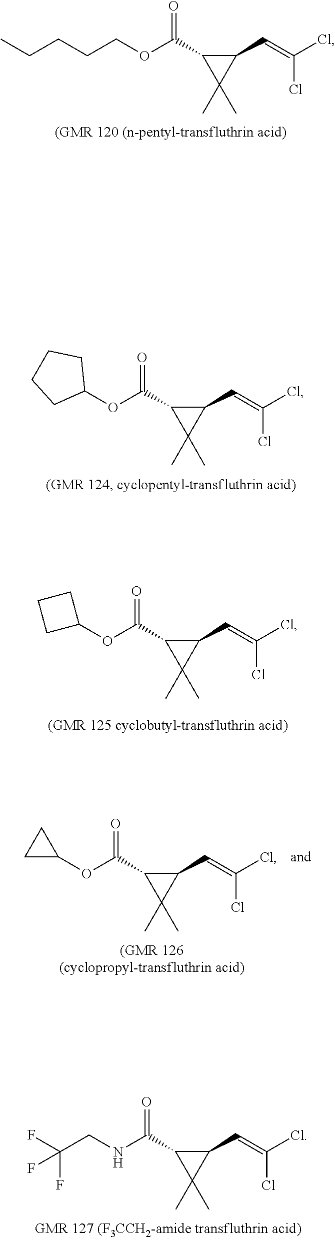 [SMILES: CCCCCOC(=O)[C@@H]1[C@@H](C=C(Cl)Cl)C1(C)C ; CC1(C)[C@H](C=C(Cl)Cl)[C@H]1C(=O)OC1CCCC1 ; CC1(C)[C@H](C=C(Cl)Cl)[C@H]1C(=O)OC1CCC1 ; CC1(C)[C@H](C=C(Cl)Cl)[C@H]1C(=O)OC1CC1 ; CC1(C)[C@H](C=C(Cl)Cl)[C@H]1C(=O)NCC(F)(F)F]

In certain embodiments, the subject matter described herein is directed to compounds of Formula II:

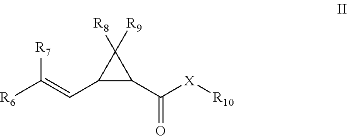

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R₆ and R₇ are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₈ and R₉ are each independently selected from hydrogen and C₁-C₆ alkyl;

R₁₀ is selected from the group consisting of hydrogen, haloalkyl, alkenyl, alkynyl, linear or branched C₁-C₁₂ alkyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, alkoxy, halo, C₁-C₆ alkyl, haloalkyl, alkenyl, alkynyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl;

R₂₇ is selected from the group consisting of hydrogen, haloalkyl, alkenyl, alkynyl, linear or branched C₁-C₁₂ alkyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl; and

In certain embodiments, X is O.

In certain embodiments, R₁₀ is selected from hydrogen, haloalkyl, alkenyl, alkynyl, C₁-C₁₂ alkyl, or C₃-C₁₂ cycloalkyl.

In certain embodiments, R₈ and R₉ are each methyl.

In certain embodiments, R₆ and R₇ are each independently selected from halo or C₁-C₃ alkyl.

In certain embodiments, the compound of Formula II is selected from the group consisting of:

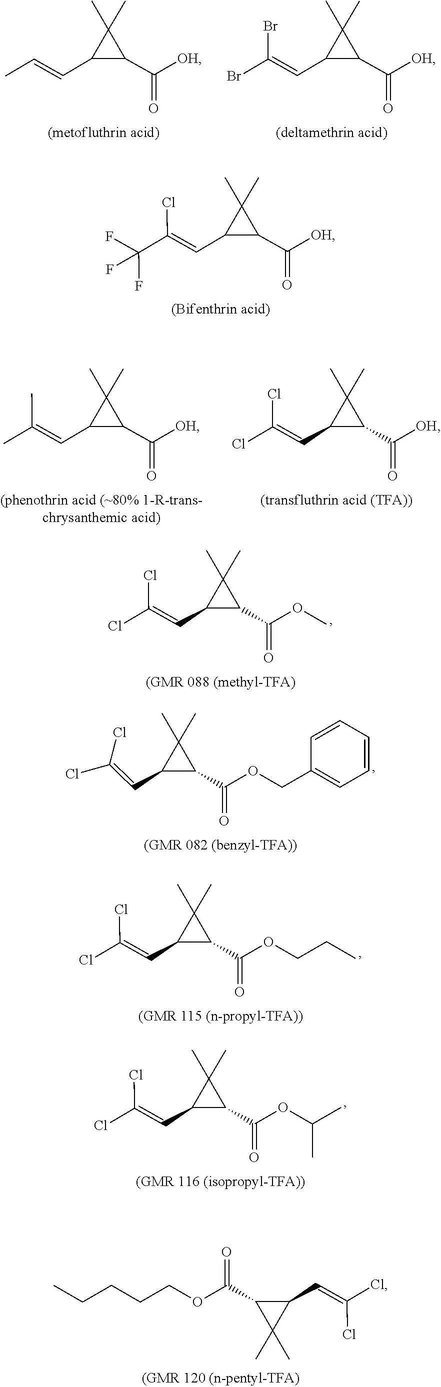

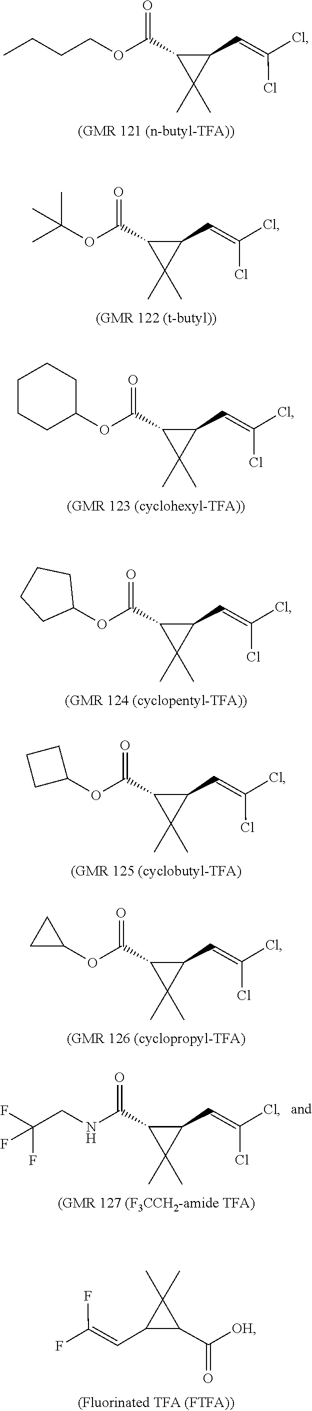

In certain embodiments, the subject matter described herein is directed to compounds of Formula III

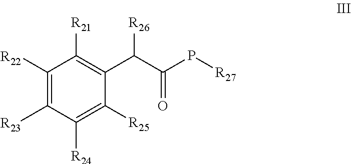

wherein R₂₁, R₂₂, R₂₃, R₂₄, and R₂₅ are each independently selected from the group consisting of hydrogen, alkoxy, halo, haloalkyl, alkenyl, alkynyl, C₁-C₆ alkyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl; and

R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

In certain embodiments, R₂₁, R₂₂, R₂₃, R₂₄, and R₂₅ are each independently selected from hydrogen and halo.

In certain embodiments, R₂₃ is halo. In certain embodiments, R₂₃ is chloro.

In certain embodiments, R₂₁, R₂₂, R₂₄, and R₂₅ are each hydrogen.

In certain embodiments, P is O.

In certain embodiments, R₂₆ is branched C₁-C₆ alkyl. In certain embodiments, R₂₆ is isopropyl.

In certain embodiments, R₂₇ is hydrogen.

In certain embodiments, the compound of Formula III is

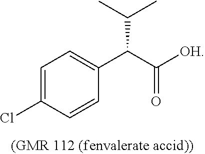

In certain embodiments, the subject matter described herein is directed to compounds of Formula IV:

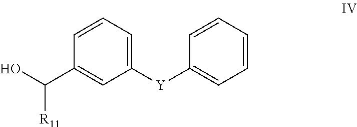

wherein Y is selected from the group consisting of O, NH, and S; and
R₁₁ is selected from the group consisting of hydrogen, C₁-C₆ alkyl, alkenyl, alkynyl, CN, halo, and haloalkyl;

In certain embodiments, Y is O.

In certain embodiments, R₁₁ is CN.

In certain embodiments, the compound of Formula IV is

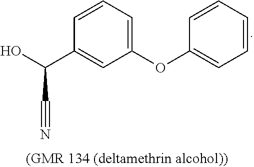 [SMILES: N#C[C@@H](O)c1cccc(Oc2ccccc2)c1]

In certain embodiments, R₁₁ is H.

In certain embodiments, the compound of Formula IV is

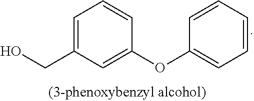 [SMILES: OCc1cccc(Oc2ccccc2)c1]

In certain embodiments, the subject matter described herein is directed to compounds of Formula V:

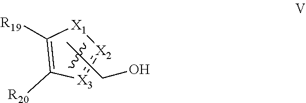

wherein R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

In certain embodiments, X₁ is O.

In certain embodiments, the double bond between X₂ and X₃ is present, wherein X₃ is C (with —COH attached), and X₂ is CH.

In certain embodiments, R₁₉ is benzyl.

In certain embodiments, the compound of Formula V is

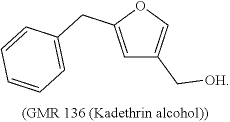 [SMILES: OCc1coc(Cc2ccccc2)c1]

In certain embodiments, X₂ is N, which is substituted by —COH.

In certain embodiments, X₁ and X₃ are each C═O, wherein the double bond between X₂ and X₃ is absent.

In certain embodiments, R₁₉ and R₂₀ are taken together with the carbon to which each is attached to form a cyclohexyl ring.

In certain embodiments, the compound of Formula V is

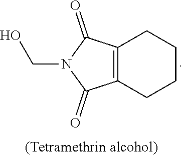 [SMILES: O=C1C2=C(CCCC2)C(=O)N1CO]

In certain embodiments, the subject matter described herein is directed to compounds of Formula VI:

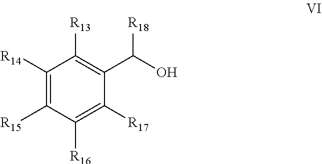

wherein R₁₃, R₁₄, R₁₅, R₁₆, and R₁₇ are each independently selected from the group consisting of hydrogen, alkoxy, halo, haloalkyl, alkenyl, alkynyl, C₁-C₆ alkyl, and C₁-C₃ alkyl-alkoxy; and

R₁₈ is selected from the group consisting of hydrogen, haloalkyl, and linear or branched C₁-C₆ alkyl.

In certain embodiments, R₁₈ is hydrogen.

In certain embodiments R₁₃, R₁₄, R₁₆, and R₁₇ are each halo. In certain embodiments R₁₃, R₁₄, R₁₆, and R₁₇ are each fluoro.

In certain embodiments, R₁₅ is hydrogen. In certain embodiments, R₁₅ is alkyl-alkoxy.

In certain embodiments, the compound of Formula VI is selected from

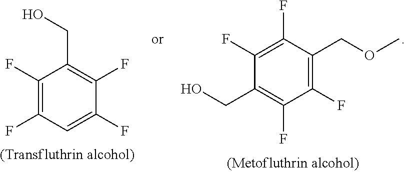

In certain embodiments, the subject matter described herein is directed to compounds of Formula VII:

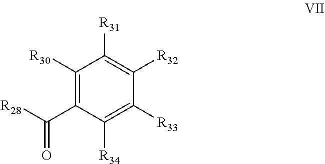

wherein R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy; and

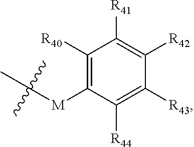

wherein M is selected from the group consisting of O, NH, and S; and

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, and hydroxy.

In certain embodiments, R₃₀, R₃₁, R₃₂, and R₃₄ are each hydrogen.

In certain embodiments, R₃₃ is

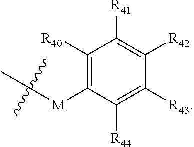

In certain embodiments, R₄₀, R₄₁, R₄₂, R₄₃, and R₄₄ are each hydrogen.

In certain embodiments, M is O.

In certain embodiments, R₂₈ is hydrogen.

In certain embodiments, the compound of Formula VII is

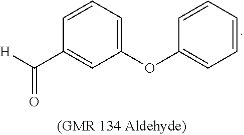 [SMILES: O=Cc1cccc(Oc2ccccc2)c1]

In certain embodiments, R₃₀, R₃₁, R₃₂, R₃₃, and R₃₄ are each hydrogen.

In certain embodiments, the subject matter disclosed herein is directed to compounds of Formula I′:

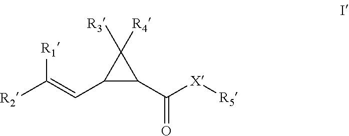

wherein R_{1′} and R_{2′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{1′} and R_{2′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{3′} and R_{4′} are each independently selected from hydrogen, haloalkyl, alkenyl, alkynyl, and C₁-C₆ alkyl;

R_{5′} is selected from the group consisting of linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl; and

X′ is selected from the group consisting of O, NH, and S.

In certain embodiments, X′ is O.

In certain embodiments, R_{3′} and R_{4′} are each methyl.

In certain embodiments, R_{5′} is linear or branched C₁-C₆ alkyl.

In certain embodiments, the subject matter disclosed herein is directed to compounds of Formula II′:

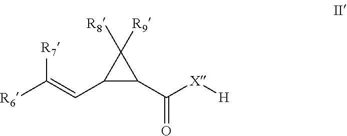

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen haloalkyl, alkenyl, alkynyl, and C₁-C₆ alkyl; and

X″ is selected from the group consisting of O, NH, and S.

In certain embodiments, X″ is O.

In certain embodiments, R_{6′} and R_{7′} are each independently selected from halo or C₁-C₆ alkyl.

In certain embodiments, R_{6′} and R_{7′} are each halo. In certain embodiments, R_{6′} and R_{7′} are each chloro.

In certain embodiments, R_{8′} and R_{9′} are each methyl.

In certain embodiments, the compound of Formula II is

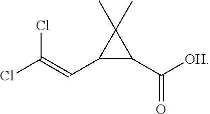

In certain embodiments, the subject matter disclosed herein is directed to compounds of Formula III′:

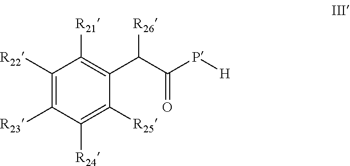

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S; and

R_{26′} is selected from the group consisting of hydrogen, haloalkyl, alkenyl, alkynyl, and linear or branched C₁-C₆ alkyl.

In certain embodiments, P′ is O.

In certain embodiments, R_{23′} is halo. In certain embodiments, R_{23′} is chloro.

In certain embodiments, R_{21′}, R_{22′}, R_{24′}, and R_{25′} are each hydrogen.

In certain embodiments, R_{26′} is branched C₁-C₆ alkyl. In certain embodiments, R_{26′} is isopropyl.

In certain embodiments, R_{27′} is hydrogen.

In certain embodiments, the compound of Formula III′ is

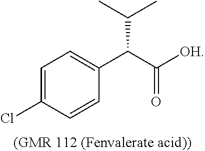

### III. Compositions

The repellent compositions described herein may take various forms, including solutions, dusts, granular formulations, and emulsions. They may be prepared from concentrates, such as emulsifiable concentrates and wettable powders. In addition to the active agents, the compositions can also comprise various inert ingredients, depending upon the form taken by the composition.

If prepared from an emulsifiable concentrate, the composition may contain a surfactant such as, for example, a mixture of a polyethylene oxide with a blend of oil soluble non-ionic and anionic sulfonates. The surfactant normally comprises between about 1 and 15% by weight of the emulsifiable concentrate.

Wettable powders can also be used to make the repellent compositions. Compositions so made may be applied to the area to be protected as emulsions in water or other liquid diluents. Typical among the carriers employed in wettable powders are walnut flour, cane sugar, fuller's earth, attapulgite clays, kaolin clays, silicas and other highly absorbent, readily wetted carriers. The wettable powders themselves generally are prepared to contain about 5 to 80% by weight of the active component, depending on the absorbency of the carrier. A wettable powder usually also contains a small amount of a surfactant.

Granular repellent compositions, wherein the active component is carried on relatively coarse particles as the carrier, are also useful in repelling crawling insect pests. Dry dusts, in which the active component is admixed with finely divided solids such as talc, attapulgite clay, kieselguhr, and other organic and inorganic solids, which act as carriers for the active component, also find utility. These finely divided solids usually have an average particle size of less than about 50 microns.

Pressurized sprays such as aerosols, in which the active component is present in solution or in a finely divided form, may also be used.

The concentration of the active agent(s) in any of the repellent compositions may vary in the range from about 0.00001% to about 10% by weight, depending on the formulation. A very broad latitude in the type of repellent composition and the concentration of the active agent(s) within the aforesaid range is possible.

In certain embodiments, the compositions described herein comprise two or more compounds or active agents wherein the combination of the two or more compounds or active agents is synergistic. In certain embodiments, the two or more compounds or active agents each independently have a concentration of about 0.1, 1.0, 2, 3, 4, 5, 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 800, or 1000 μg/cm².

In certain embodiments, the subject matter disclosed herein is directed to a compound of Formula I, in intimate admixture with a carrier.

In certain embodiments, the compositions disclosed herein comprise a compound of Formula I and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, wherein the composition is synergistic, in intimate admixture with a carrier, in any molar or weight ratio.

In certain embodiments, the compositions disclosed herein comprise about 0.01-99.99%, 1-99%, 5-95%, 10-90%, 20-80%, 30-70%, 40-60%, or 50% of a compound of Formula II or Formula III and 99.99-0.01%, 99-1%, 95-5%, 90-10%, 80-20%, 70-30%, 60-40%, or 50% of a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, wherein the composition is synergistic, in intimate admixture with a carrier. In certain embodiments, the compound of Formula II or Formula III comprises about 1%, 2%, 3%, 4%, 5%, 10%, 15%, 20%, 2%5, 30%, 40%, 50%, 60%, 65%, 70%, 75%, 80%, 90%, 95%, 96%, 97%, or 98% of the composition in combination with a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, and VUAA-1, wherein the composition is synergistic, in intimate admixture with a carrier.

In certain embodiments, the compositions disclosed herein comprise a compound of Formula II or Formula III and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, wherein the composition is synergistic, in intimate admixture with a carrier, in any molar or weight ratio.

In certain embodiments, the compositions disclosed herein comprise about 0.01-99.99%, 1-99%, 5-95%, 10-90%, 20-80%, 30-70%, 40-60%, or 50% of a compound of Formula II′ or Formula III′ and 99.99-0.01%, 99-1%, 95-5%, 90-10%, 80-20%, 70-30%, 60-40%, or 50% of a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, and VUAA-1, wherein the composition is synergistic, in intimate admixture with a carrier.

In certain embodiments, the compound of Formula II′ or Formula III′ comprises about 1%, 2%, 3%, 4%, 5%, 10%, 15%, 20%, 2%5, 30%, 40%, 50%, 60%, 65%, 70%, 75%, 80%, 90%, 95%, 96%, 97% or 98% of the composition in combination with a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, in intimate admixture with a carrier.

In certain embodiments, the compositions disclosed herein comprise a compound of Formula II′ or a compound of Formula III′ a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, and VUAA-1, wherein the composition is synergistic, in intimate admixture with a carrier, in any weight or molar ratio.

In certain embodiments, the compositions disclosed herein comprise about 0.01-99.99%, 1-99%, 5-95%, 10-90%, 20-80%, 30-70%, 40-60%, or 50% of a compound of Formula IV, Formula V, or Formula VI, and 99.99-0.01%, 99-1%, 95-5%, 90-10%, 80-20%, 70-30%, 60-40%, or 50% of a compound of Formula II′ or Formula III′, wherein the composition is synergistic, in intimate admixture with a carrier. In certain embodiments, the compound of Formula IV, Formula V, or Formula VI comprises about 1%, 2%, 3%, 4%, 5%, 10%, 15%, 20%, 2%5, 30%, 40%, 50%, 60%, 65%, 70%, 75%, 80%, 90%, 95%, 96%, 97%, or 98% of the composition in combination with a compound of Formula II′ or Formula III′, wherein the composition is synergistic, in intimate admixture with a carrier.

In certain embodiments, the compositions disclosed herein comprise a compound of Formula IV, Formula V, or Formula VI, and a compound of Formula II′ or Formula III′, wherein the composition is synergistic, in intimate admixture with a carrier, in any weight or molar ratio.

In certain embodiments, the compositions disclosed herein comprise about 0.01-99.99%, 1-99%, 5-95%, 10-90%, 20-80%, 30-70%, 40-60%, or 50% of a compound of Formula VII and about 99.99-0.01%, 99-1%, 95-5%, 90-10%, 80-20%, 70-30%, 60-40%, or 50% of a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, a compound of Formula II′, a compound of Formula III′, a compound of Formula IV, a compound of Formula V, and a compound of Formula VI, wherein the composition is synergistic, in intimate admixture with a carrier. In certain embodiments, the compound of Formula VII comprises about 1%, 2%, 3%, 4%, 5%, 10%, 15%, 20%, 2%5, 30%, 40%, 50%, 60%, 65%, 70%, 75%, 80%0, 90%, 95%, 96%, or 98% of the composition in combination with a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, and VUAA-la compound of Formula II′, a compound of Formula III′, a compound of Formula IV, a compound of Formula V, and a compound of Formula VI.

In certain embodiments, the compositions disclosed herein comprise a compound of Formula VII and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, a compound of Formula II′, a compound of Formula III′, a compound of Formula IV, a compound of Formula V, and a compound of Formula VI, wherein the composition is synergistic, in intimate admixture with a carrier, in any molar or weight ratio.

In certain embodiments, the compositions disclosed herein comprise about 0.01-99.99%, 1-99%, 5-95%, 10-90%, 20-80%, 30-70%, 40-60%, or 50% of a compound of Formula I′ and about 99.99-0.01%, 99-1%, 95-5%, 90-10%, 80-20%, 70-30%, 60-40%, or 50% of a compound of Formula II′ or Formula III′, wherein the composition is synergistic, in intimate admixture with a carrier. In certain embodiments, the compound of Formula I′ comprises about 1%, 2%, 3%, 4%, 5%, 10%, 15%, 20%, 2%5, 30%, 40%, 50%, 60%, 65%, 70%, 75%, 80%, 90%, 95%, 96%, or 98% of the composition in combination with a compound of Formula II′ or III′, wherein the composition is synergistic, in intimate admixture with a carrier.

In certain embodiments, the compositions disclosed herein comprise a compound of Formula I′ and a compound of Formula II′ or Formula III′, wherein the composition is synergistic, in intimate admixture with a carrier, in any molar or weight ratio.

In certain embodiments, the compositions disclosed herein comprise two compounds of Formula II, two compounds of Formula III, or one compound of Formula II and one compound of Formula III, and a carrier, wherein the composition is synergistic.

In certain embodiments, the compositions disclosed herein comprise about 0.01-99.99%, 1-99%, 5-95%, 10-90%, 20-80%, 30-70%, 40-60%, or 50% of an essential oil; and about 99.99-0.01%, 99-1%, 95-5%, 90-10%, 80-20%, 70-30%, 60-40%, or 50% of a compound selected from the group consisting of a pyrethroid, a compound of Formula II, Formula III, Formula IV, Formula V, Formula VI, and Formula VII, wherein the composition is synergistic, in intimate admixture with a carrier.

In certain embodiments, the compositions disclosed herein comprise an essential oil and a compound selected from the group consisting of a pyrethroid, a compound of Formula II, a compound of Formula III, a compound of Formula IV, a compound of Formula V, a compound of Formula VI, and a compound of Formula VII, wherein the composition is synergistic, in intimate admixture with a carrier, in any molar or weight ratio. In certain embodiments of this embodiment, the essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil. In certain embodiments of this embodiment, the essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil. In certain embodiments of this embodiment, wherein said second compound is a pyrethroid, said pyrethroid is selected from the group consisting of permethrin, tetramethrin, metofluthrin, bifenthrin, kadethrin, allethrin, cyfluthrin, deltamethrin, empenthrin, cypermethrin, cyphenothrin, esfenvalerate, fenvalerate, flumethrin, tefluthrin, phenothrin, and transfluthrin. In certain embodiments of this embodiment, wherein said second compound is a pyrethroid, said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.

In certain embodiments, the subject matter disclosed herein is directed to a composition comprising about 0.01-99.99%, 1-99%, 5-95%, 10-90%, 20-80%, 30-70%, 40-60%, or 50%, of a compound of Formula II, a compound of Formula II′, a compound of Formula III′, or a compound of Formula III; and about 99.99-0.01%, 99-1%, 95-5%, 90-10%, 80-20%, 70-30%, 60-40%, or 50% of a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, wherein the composition is synergistic, in intimate admixture with a carrier.

“Synergistic” as used herein refers to a combination which is more effective than the additive effects of the two or more single agents. A determination of a synergistic interaction between two active compounds may, in some embodiments, be based on the results obtained from the experimental information described herein. The results of these experiments can be analyzed using, for example, the Bliss activity method or co-toxicity factors (CF), described further herein.

### IV. Methods of Repelling Arthropods

In certain embodiments, the subject matter described herein is directed to a method of repelling an arthropod from an object or locus, comprising contacting said object or locus with any compound or composition disclosed herein. In certain embodiments, the arthropod is repelled from an adjacent area to said locus. In embodiments, the arthropod is not physically contacted with said object or locus.

In certain embodiments, the subject matter described herein is directed to a method of repelling arthropods, comprising contacting an object or locus with any compound or composition disclosed herein, wherein the arthropods are repelled from an adjacent area to said object or locus, wherein the arthropods are not physically contacted with the object or locus.

In certain embodiments, the subject matter described herein is directed to a method of providing any compound or composition disclosed herein in an arthropod repellent medium from which a vaporized compound or composition disclosed herein can be dispersed. Non-limiting examples of suitable media include alcohols such as ethanol, glycerin and polyethylene glycol; ketones such as acetone; ethers such as tetrahydrofuran and dioxane; aliphatic hydrocarbons or petroleum distillates such as gasoline, naphtha, mineral spirits, tar, hexane, kerosene, toluene, xylene, limonene, turpentine, paraffin and petroleum benzene; esters such as ethyl acetate; and essential oils such as pine oil or citronella oil,

In certain embodiments, vaporization of the repellent may be assisted by thermal volatilization. Thermal volatilization may proceed by flame, ionizing radiation, oven, sunlight, electrical pulse, laser, gas heating element, or electric-powered heating element, such as induction heating, chemical reaction, microwave irradiation, ultrasound or a mixture thereof.

In certain embodiments, the compositions or compounds disclosed herein can formulated for use in a vaporizer, evaporator, fan, heat, candle, or wicked apparatus.

The spatial arthropod repellent compounds disclosed herein may be formulated into any suitable composition to dispense a suitable amount of the repellent compound into an environmental area in which it is desired to repel arthropods, wherein the arthropods do not come into contact with the locus. This repellency effective amount would typically range from about 1 mg/cm² down to about 1 μg/cm² or from about 100 g/cm² down to about 30 ng/cm² of the base area of the environmental area in which repellency is to be sought. In certain embodiments, the spatial arthropod repellent compounds disclosed herein may be formulated into any suitable composition to dispense a suitable amount of the repellent compound onto a locus, wherein the insects are repelled at least 1 cm, 2 cm, 1 in, 2 in, 3 in, 4 in, 5 in, 6 in, 7 in, 8 in, 9 in, 10 in, 11 in, 1 foot, 2 feet, 3 feet, 4 feet, 5 feet, 6 feet, 7 feet, 8 feet, 9 feet, 10 feet, 11 feet, 12 feet, 13 feet, 14 feet, 15 feet, 16 feet, 17 feet, 18 feet, 19 feet, 20 feet, 21 feet, 22 feet, 23 feet, 24 feet, or 25 feet from the locus. In certain embodiments, arthropod repellent compounds disclosed herein are effective both in the vapor phase as spatial repellents and on contact.

In certain embodiments, the subject matter disclosed herein is directed to methods for repelling one or more insects using the compositions described herein.

In repelling insect pests according to the methods, the locus from which such insect pests are to be repelled will be contacted with a repellent amount of the compound of Formula I, Formula II, Formula III, Formula IV, Formula V, Formula VI, Formula VII, Formula I′, Formula II′, Formula III′, or a synergistic composition. A repellent amount of the compound of Formula I Formula II, Formula III, Formula IV, Formula V, Formula VI, Formula VII, Formula I′, Formula II′, Formula III′, or a synergistic composition can vary somewhat, depending on the species of insect to be repelled, the nature of the locus, including the type of surface, from which the insects are to be repelled, and so forth, but generally between about 1 mg/m² and 200 mg/m² is a repellent amount. As is well known in the art, the degree of effectiveness of the repellent may vary with the formulation and the method of application.

In carrying out the methods, the compound of Formula I, Formula II, Formula III, Formula IV, Formula V, Formula VI, Formula VII, Formula I′, Formula II′, Formula III′, or a synergistic composition may be applied in any suitable fashion to the area in which the repellency of insect pests, including acarids, is desired. Means of effective applications are well known in the art.

In certain embodiments, the subject matter disclosed herein is directed to a method of repelling a species of insect from a locus to which said species of insect is normally attracted, comprising contacting on said locus a non-toxic effective insect repellent composition comprising a compound of Formula II′ or a compound of Formula III′ in an amount of about 0.01-99.99%, 1-99%, 5-95%, 10-90%, 20-80%, 30-70%, 40-60%, or 50%; and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1 in an amount of about 99.99-0.01%, 99-1%, 95-5%, 90-10%, 80-20%, 70-30%, 60-40%, or 50%, wherein the composition is synergistic, in intimate admixture with a carrier.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula II′ or a compound of Formula III′.

In certain embodiments of the methods described herein, the compositions comprise two or more compounds or active agents wherein the combination of the two or more compounds or active agents is synergistic. In certain embodiments, the two or more compounds or active agents each independently have a concentration of about 0.1, 1.0, 2, 3, 4, 5, 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 800, or 1000 μg/cm².

In certain embodiments, the subject matter disclosed herein is directed to a method of repelling a species of insect from a locus to which said species of insect is normally attracted, comprising contacting on said locus a non-toxic effective insect repellent composition comprising a compound of Formula IV, a compound of Formula V, or a compound of Formula VI in an amount of about 0.01-99.99%, 1-99%, 5-95%, 10-90%, 20-80%, 30-70%, 40-60%, or 50%; and a compound of Formula II′ or a compound of Formula III′ in an amount of about 99.99-0.01%, 99-1%, 95-5%, 90-10%, 80-20%, 70-30%, 60-40%, or 50%, wherein the composition is synergistic, in intimate admixture with a carrier.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula IV.

In certain embodiments, the subject matter disclosed herein is directed to a method of repelling a species of insect from a locus to which said species of insect is normally attracted, comprising contacting on said locus a non-toxic effective insect repellent composition comprising a compound of Formula VII in an amount of about 0.01-99.99%, 1-99%, 5-95%, 10-90%, 20-80%, 30-70%, 40-60%, or 50%; and a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, and VUAA-1, a compound of Formula II′, a compound of Formula III′, a compound of Formula IV, a compound of Formula V, and a compound of Formula VI in an amount of about 99.99-0.01%, 99-1%, 95-5%, 90-10%, 80-20%, 70-30%, 60-40%, or 50%, wherein the composition is synergistic, in intimate admixture with a carrier.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula VII.

In certain embodiments, the subject matter disclosed herein is directed to a method of repelling a species of insect from a locus to which said species of insect is normally attracted, comprising contacting on said locus a non-toxic effective insect repellent composition comprising a compound of Formula I′ in an amount of about 0.01-99.99%, 1-99%, 5-95%, 10-90%, 20-80%, 30-70%, 40-60%, or 50%, and a compound of Formula II′ or a compound of Formula III′ in an amount of about 99.99-0.01%, 99-1%, 95-5%, 90-10%, 80-20%, 70-30%, 60-40%, or 50%, wherein the composition is synergistic, in intimate admixture with a carrier.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula I′.

In certain embodiments, the subject matter described herein is directed to a method of repelling an arthropod from a locus to which said arthropod is normally attracted, comprising exposing said arthropod to any single compound described herein. In certain embodiments, the arthropod is exposed to said single compound in the form of a vapor active repellent. In certain embodiments, the benzaldehyde derivatives in Table 8 are repellents. In certain embodiments, the benzaldehyde derivatives in Table 8 are specifically vapor active repellents.

In certain embodiments, the arthropods repelled by the compounds, compositions, or methods disclosed herein are insects. In certain embodiments, the insects repelled by the compounds, compositions, or methods disclosed herein are Hemiptera Insects, selected from Delphacidae (planthoppers), such as Laodelphax striatellus (small brown planthopper), Nilaparvata hugens (brown planthopper), Sogatella furcifera (white-backed rice planthopper); Deltocephalidae (leafhoppers), such as Nephotettix cincticeps (green rice leafhopper), Recilia dorsalis (zig-zag rice leaf hopper), Nephotettix virescens (green rice leafhopper); Aphididae (aphids), stink bugs, Aleyrodidae (whiteflies), scales, Tingidae (lace bugs), or Psyllidae (suckers).

In certain embodiments, the insects repelled by the compounds, compositions, or methods disclosed herein are Lepidoptera Insects, selected from Pyralidae, such as Chilo suppressalis (rice stem borer), Cnaphalocrocis medinalis (rice leafroller), Plodia interpunctella (Indian meal moth); Noctuidae, such as Spodoptera litura (tobacco cutworm), Pseudaletia separata (rice armyworm), Mamestra brassicae (cabbage armyworm); Pieridae, such as Pieris rapae crucivora (common cabbageworm); Tortricidae, such as Adoxophyes spp.; Carposinidae; Lyonetiidae; Lymantriidae; Plusiinae; Agrotis spp. such as Agrotis segetum (turnip cutworm), or Agrotis ipsilon (black cutworm); Helicoverpa spp.; Heliothis spp.; Plutella xylostella; Parnara guttata (rice skipper); Tinea pellionella (casemaking clothes moth); or Tineola bisselliella (webbing clothes moth).

In certain embodiments, the insects repelled by the compounds, compositions, or methods disclosed herein are Diptera Insects, Culex spp., such as Culex pipiens pallens (common mosquito), Culex tritaeniorhynchus, Aedes spp., such as Aedes aegypti, Aedes albopictus; Anopheles spp., such as Anopheles sinensis; Chironomidae (midges); Muscidae, such as Musca domestica (housefly), Muscina stabulans (false stablefly), Fannia canicularis (little housefly); Calliphoridae; Sarcophagidae; Anthomyiidae, such as Delia platura (seedcorn maggot), Delia antiqua (onion maggot); Tephritidae (fruit flies); Drosophilidae; Psychodidae (moth flies); Tabanidae; Simuliidae (black flies); Stomoxyidae (stable flies); Phoridae; or Ceratopogonidae (biting midges).

In certain embodiments, the insects repelled by the compounds, compositions, or methods disclosed herein are Coleoptera Insects (Beetles), several nonlimiting examples of which include Corn rootworms, such as Diabrotica virgifera (western corn rootworm), Diabrotica undecimpunctata howardi (southern corn rootworm); Scarabaeidae (scarabs), such as Anomala cuprea (cupreous chafer), Anomala rufocuprea (soybean beetle); Curculionidae (weevils), such as Sitophilus zeamais (maize weevil), Lissorhoptrus oryzophilus (ricewater weevil), ball weevil, Callosobruchus chinensis (adzuki bean weevil); Dermestidae, such as Authrenus verbasci (varied carpet beetle), Attagenus unicolor japonicus (black carpet beetle); Tenebrionidae (darkling beetles), such as Tenebrio molitor (yellow mealworm), or Tribolium castaneum (red flour beetle); Chrysomelidae (leaf beetles) such as Oulema oryzae (rice leaf beetle), Phyllotreta striolata (striped flea beetle), Aulacophora femoralis (cucurbit leaf beetle); Anobiidae; Epilachna spp. such as Epilachna vigintioctopunctata (twenty-eight-spotted ladybird); Lyctidae (powderpost beetles), Bostrychidae (false powderpost beetles), or Cerambycidae, Paederus fuscipes (robe beetle).

In certain embodiments, the insects repelled by the compounds, compositions, or methods disclosed herein are Dictyoptera Insects, such as the following: Blattella germanica (German cockroach); Periplaneta filiginosa (smokybrown cockroach); Periplaneta americana (American cockroach); Periplaneta brunnea (brown cockroach); or Blatta orientalis (oriental cockroach).

In certain embodiments, the insects repelled by the compounds, compositions, or methods disclosed herein are Thysanoptera Insects (Thrips), such as Thrips palmi, Flankliniella occidentalis (western flower thrips), or Thrips hawaiiensis (flower thrips).

In certain embodiments, the insects repelled by the compounds, compositions, or methods disclosed herein are Hymenoptera Insects, such as Formicidae (ants); Vespidae (hornets); Polistes spp. (long-legged wasps); Bethylidae; or Tenthredinidae (sawflies), such as Athalis rosae ruficornis (cabbage sawfly).

In certain embodiments, the insects repelled by the compounds, compositions, or methods disclosed herein are Orthoptera Insects, such as Gryllotalpidae (mole crickets); or Acrididae (grasshoppers).

In certain embodiments, the insects repelled by the compounds, compositions, or methods disclosed herein are Siphonaptera Insects (Fleas), such as Ctenocephalides canis (dog flea); Ctenocephalides felis (cat flea); or Pulex irritans.

In certain embodiments, the insects repelled by the compounds, compositions, or methods disclosed herein are Anoplura Insects (Lice), such as Pediculus corporis (body louse); Pediculus humanus (head louse); or Pthirus pubis (crab louse).

In certain embodiments, the insects repelled by the compounds, compositions, or methods disclosed herein are Isoptera Insects, such as Reticulitermes speratus; Coptotermes formosanus (Formosan subterranean termite).

In certain embodiments, the insects repelled by the compounds, compositions, or methods disclosed herein are Harmful Acarina, such as Ixodidae (Ticks): Boophilus microplus; Haemaphysalis longiconis Tetranychidae (spider mites): Tetranychus cinnabarimus (carmine spider mite); Tetranychus urticae (two-spotted spider mite); Tetranychus kanzawai (Kanzawa spider mite); Panonychus citri (citrus red mite); Panonychus ulmi (European red mite); House-dust Mites: Acaridae such as Tyrophagus putrescentiae (copra mite), Aleuroglyphus ovatus (brown legged grain mite); Dermanyssidae such as Dermatophagoides farinae (American house dust mite), Dermatophagoides pteronyssinus; mites parasitizing honeybees, such as Varroa jacobsoni; Euvarroa sinhai, Acarapis woodi; Tropilaelaps clareae; Glycyphagidae, such as Glycyphagus privatus, Glycyphagus domesticus, Glycyphagus destructor; Cheyletidae, such as Chelacaropsis malaccensis, Cheyletus fortis; Tarsonemidae; Chortoglyphus spp.; Haplochthonius spp. Chilognatha (millipedes), such as Oxydus spp.; Chilopoda (centipedes), such as red centipede; wood lice, such as Porcellio spp., Porcellionides spp.; and pill bugs, such as Armadillidium spp.

In certain embodiments, the arthropod repelled by the compounds, compositions, or methods disclosed herein is selected from the group consisting of a fly, spider, butterfly, crab, mosquito, centipede, tick, millipede, and scorpion. In certain embodiments, the arthropod repelled by the compounds, compositions, or methods disclosed herein is selected from the group consisting of a fly, spider, butterfly, crab, mosquito, centipede, tick, millipede, scorpion, roache, ant, termite, silverfish, and wasp. In certain embodiments, the arthropod is an insect.

In certain embodiments, the insect is a mosquito. In certain embodiments, the mosquito genera is selected from the group consisting of Culex, Anopheles, and Aedes. In certain embodiments, the mosquito is of the species, Aedes aegypti.

In certain embodiments, the subject matter described herein is directed to a method of controlling one or more insects, comprising contacting the insects with a synergistic composition comprising a compound of Formula II′ or a compound of Formula III′ in an amount of about 0.01-99.99%, 1-99%, 5-95%, 10-90%, 20-80%, 30-70%, 40-60%, or 50% and a pyrethroid in an amount of about 99.99-0.01%, 99-1%, 95-5%, 90-10%, 80-20%, 70-30%, 60-40%, or 50%, wherein the synergistic composition produces, when the insects are brought into contact with the synergistic composition, a combined toxicant effect greater than the sum of the separate toxicant effects from the compound of Formula II′ or Formula III′ and the pyrethroid, at comparable concentrations.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula II′ or Formula III′.

In certain embodiments, the subject matter described herein is directed to a method of repelling a species of insect from a locus to which said species of insect is normally attracted, comprising contacting on said locus a non-toxic effective insect repellent composition comprising an essential oil in an amount of about 0.01-99.99%, 1-99%, 5-95%, 10-90%, 20-80%, 30-70%, 40-60%, or 50%, and a second compound in an amount of about 99.99-0.01%, 99-1%, 95-5%, 90-10%, 80-20%, 70-30%, 60-40%, or 50%, selected from the group consisting of a pyrethroid, a compound of Formula II, Formula III, Formula IV, Formula V, Formula VI, and Formula VII, wherein the composition is synergistic, in intimate admixture with a carrier.

As used herein, “controlling one or more insects” or “controlling one or more arthropods” refers to mitigating or reducing a population of insects or a population of arthropods, respectively. In certain embodiments, the synergistic methods disclosed herein achieve a combined toxicant effect greater than 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% than the sum of the separate toxicant effects from the compound of Formula II′ or Formula III′ and the second compound, at comparable concentrations.

In certain embodiments, the toxicant effect refers to the vapor toxicity of the synergistic composition.

### V. Methods of Preparing Compounds and Agrochemically Acceptable Salts Thereof

Flash column chromatography (CombiFlash Rf 200i) was performed using silica gel (RediSep Rf Gold Normal-Phase Silica) purchased from TELEDYNE ISCO. Thin layer chromatography (TLC) was performed on silica gel matrix on TLC Al foils with fluorescent indicator 254 nm, purchased from Sigma-Aldrich. All hexane used was a mixture of hexanes. Reagents were purchased from Sigma-Aldrich and were used without purification, unless otherwise noted.

Exemplary Methods for Preparing Compounds of Formula I

Method A. Briefly, thionyl chloride (1.4 equiv) was added dropwise to a solution of carboxylic acid (1 equiv) in methanol (5 mL/mmol). The mixture was vigorously stirred and monitored by TLC. Upon completion, the crude mixture was concentrated under reduced pressure with a rotoevaporator and purified on a silica gel column (Tetrahedron 69 (2013) 10946-10954).

Method B. Alcohol (1 equiv), carboxylic acid (1.2 equiv) and 4-dimethylaminopyridine (0.15) were dissolved in dichloromethane (1 mL/mmol) and cooled to 0° C. N,N-dicyclohexylcarbodiimide (1.05 equiv) in dichloromethane (2 mL/mmol) was added dropwise over 5 minutes, and the mixture was stirred for 0.25 hours at 0° C. The mixture was warmed to room temperature and stirred for 16 hours. The mixture was cooled at 0° C. for one hour and then filtered. The filtrate was washed with hexane, and the mother liquor was collected and concentrated in vacuo. The crude mixture was placed directly on and purified by silica gel column chromatography (Klimavicz et al. 2018).

Method C. Carboxylic acid (1 equiv) was added to a suspension of potassium carbonate (1.21 equiv) in acetone (1.6 mL/mmol) at room temperature and stirred for 0.5 hours. The mixture was cooled to 0° C., alkyl halide (1.53 equiv) was added, and the reaction was refluxed overnight for 16 hours. The mixture was cooled to room temperature and filtered through a celite plug. The mother liquor was concentrated under reduced pressure and purified by silica gel chromatography (G. Esteban et al. Tetrahedron 54 (1998) 197-212). Compositions comprising two or more compounds can be prepared by contacting the two or more compounds, optionally in the presence of a suitable carrier.

Exemplary Procedure for the Hydrolysis of Pyrethroids

Method D. Pyrethroid (1 equiv) was suspended in NaOH_{(aq)} or KOH_{(aq)} (1M, 5 equiv) and methanol (10 mL/mmol) and refluxed for 16 hours. The mixture was cooled to room temperature, concentrated by rotoevaporation, and extracted with ethyl acetate. The organic layer obtained through extraction from the basic aqueous layer was collected, dried with magnesium sulfate, filtered, concentrated in vacuo, and purified by silica gel column chromatography to afford the hydrolyzed alcohol. The basic aqueous layer was then collected and acidified with 1M HCl. The now acidic aqueous layer was then extracted with ethyl acetate. The organic layer obtained through extraction from the acidic aqueous layer was collected, dried with magnesium sulfate, filtered, concentrated in vacuo, and purified by silica gel column chromatography to afford the hydrolyzed carboxylic acid.

Method E. KOH (10 equiv) was added to a solution of pyrethroid (1 equiv) in MeOH/H₂O (5 mL/mmol) and stirred for 16 hours at room temperature. The mixture was concentrated by rotoevaporation, and extracted with ethyl acetate. The organic layer obtained through extraction from the basic aqueous layer was collected, dried with magnesium sulfate, filtered, concentrated in vacuo, and purified by silica gel column chromatography to afford the hydrolyzed alcohol. The basic aqueous layer was then collected and acidified with 1M HCl. The now acidic aqueous layer was then extracted with ethyl acetate. The organic layer obtained through extraction from the acidic aqueous layer was collected, dried with magnesium sulfate, filtered, concentrated in vacuo, and purified by silica gel column chromatography to afford the hydrolyzed carboxylic acid.

Exemplary Procedure for Conversion of Cyanohydrin to Aldehyde and Carboxylic Acid

Method F. KOH (5 equiv) was added to a solution of cyanohydrin (1 equiv) in 1/1 Dioxane/H₂O (10 mL/mmol), and the solution was stirred for 16 hours. The mixture was extracted with ethyl acetate. The organic layer obtained through extraction from the basic aqueous layer was collected, dried with magnesium sulfate, filtered, concentrated in vacuo, and purified by silica gel column chromatography to afford the aldehyde. The basic aqueous layer was then collected and acidified with 1M HCl_{(aq)}. The now acidic aqueous layer was then extracted with ethyl acetate. The organic layer obtained through extraction from the acidic aqueous layer was collected, dried with magnesium sulfate, filtered, concentrated in vacuo, and purified by silica gel column chromatography to afford the hydrolyzed carboxylic acid.

Compounds can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database). DTT refers to dithiothreitol. DHAA refers to dehydroascorbic acid.

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I, Formula II, Formula III, Formula IV, Formula V, Formula VI, Formula VII, Formula I′, Formula II′, or Formula III′ may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or agrochemically acceptable salts thereof.

Compositions comprising two or more compounds can be prepared simply by contacting one with the other.

The General Procedures and Examples provide exemplary methods for preparing compounds. Those skilled in the art will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

### VI. Kits

In another aspect, described herein are articles of manufacture, for example, a “kit,” containing materials useful for repelling one or more insects. According to one embodiment, a kit may comprise (a) a first container with a compound of Formula II contained therein, such as transfluthrin acid; and (b) a second container comprising a second compound, such as DEET, contained therein.

The kit may further comprise a label or package insert, on or associated with the containers. The term “package insert” is used to refer to instructions customarily included in commercial packages of insect repellent products, that contain information about the usage, applications, and/or warnings concerning the use of such insect repellent products. The kit may include an applicator for spreading the compounds onto a surface. The label or package insert indicates that the composition is used for repelling the insect of choice, such as mosquitos.

The kit may further comprise directions for the application of the compound of Formula II and the second compound. For example, the kit may further comprise directions for the simultaneous, sequential or separate application of the first and second compounds.

In certain other embodiments, wherein the kit comprises a composition of Formula II and a second compound, such as DEET, the kit may comprise a container for containing the separate compositions such as a divided bottle or a divided foil packet, however, the separate compositions may also be contained within a single, undivided container. Typically, the kit comprises directions for the application of the separate components. The kit form is particularly advantageous when the separate components are preferably applied in separate steps (e.g., not in mixed compositions), but separately on a surface, side-by-side.

### VII. Pyrethroids

As used herein, non-limiting examples of pyrethroids include permethrins, tetramethrin, metoflurthrin, bifenthrin, kappa-bifenthrin, kadethrin, allethrin, bioallethrin, cyfluthrin, beta-cyfluthrin, deltamethrin, cypermethrin, alpha-cypermethrin, beta-cypermethrin, theta-cypermethrin, zeta-cypermethrin, cyphenothrin, esfenvalerate, fenvalerate, flumethrin, tefluthrin, kappa-tefluthrin, phenothrin, etofenprox, fluvalinate, acrinathrin, halfenprox, flubrocythrinate, bioethanomethrin, brofenvalerate, brofluthrinate, bromethrin, butethrin, chlorempenthrin, cylethrin, cycloprothrin, cyhalothrin, gamma-cyhalothrin, lambda-cyhalothrin, dimethfluthrin, dimethrin, empenthrin, chloroprallethrin, fenfluthrin, fenpirithrin, fenpropathrin, flucythrinate, fluvalinate, tau-fluvalinate, furamethrin, furethrin, heptafluthrin, imiprothrin, japothrins, methothrin, metofluthrin, epsilon-metofluthrin, momfluorothrin, epsilon-momfluorothrin, pentmethrin, biopermethrin, transpermethrin, profluthrin, proparthrin, pyresmethrin, renofluthrin, meperfluthrin, resmethrin, bioresmethrin, cismethrin, terallethrin, tetramethylfluthrin, tralocythrin, tralomethrin, valerate, flufenprox, halfenprox, protrifenbute, silafluofen, sulfoxime, thiofluoximate, and transfluthrin.

### VIII. Essential Oils

As used herein, non-limiting examples of essential oils include Almond Oil Bitter, Almond Oil Sweet, Amyris Oil W.I., Angelica Root Oil, Angelica Seed Oil, Anise Oil Chinese, Anise Oil Spanish, Anise Oil Vietnam, Aniseed Oil, Aniseed Oil Terpenes, Apricot Kernel Oil, Armoise Oil, Artemesia Afra, Asafoetida Oil, Avocado Oil, Balsam Copaiba, Balsam Copaiba Oil, Balsam Fir Canada, Balsam Peru, Balsam Peru Oil, Balsam Tolu PG, Basil Oil Comores Type, Basil Oil Indian, Basil Oil Linalool Type, Bay Oil Terpeneless Type, Bay Oil W.I., Bay Oil W.I. Bleached & Filtered, Bay Terpenes PG, Beeswax Absolute, Benzoin Siam Resin, Bergamot Oil, Bergamot Oil B.F., Bergamot Oil Residues, Bergamot Oil Terpenes, Birch Tar Crude, Birch Tar Rectified, Black Currant Bud Absolute, Blood Orange Oil, Bois de Rose Oil, Boronia Absolute Tasmanian, Brominated Vegetable Oil, Buchu Leaf Oil Betulina, Buchu Leaf Oil Crenulata, Buchu Oil, Sulfur Fractions 40%, Cabreuva Oil, Cade Oil, Cade Oil Rectified, Cajeput Oil, Calamus Oil, Camphor Oil 1.070, Camphor Oil Chinese, Camphor Oil White Taiwan, Cananga Oil, Caraway Oil, Cardamom Oil, Carrot Seed Oil, Carvene, Cascarilla Bark Oil, Cassia Oil, Catnip Oil, Cedarleaf Oil, Cedarwood Oil (i.e. Cedarwood Oil Atlas, Cedarwood Oil Chinese, Cedarwood Oil Texas, Cedarwood Oil Texas Redistilled, and Cedarwood Oil Virginiana), Celery Seed Oil, Chamomile Oil Blue, Chamomile Oil Roman, Chamomile Oil Wild Maroc, Cinnamon Bark Oil, Cinnamon Leaf Oil Ceylon, Cistus Absolute, Cistus Oil, Citronella Oil Java 85/35, Citronella Oil Chinese 85/35, Citronella Oil Ceylon, Citronella Terpenes, Clove Bud Indonesian, Clove Bud Oil Prime, Clove Leaf Oil Indonesian 70/80, Clove Leaf Oil Madagascar 80/82, Clove Leaf Oil Redistilled, Clove Stem Oil, Clove Terpenes, Coffee Oil, Cognac Oil Green, Cognac Oil White, Coriander Herb Oil (Cilantro), Coriander Seed Oil, Cornmint Oil 50% Dementh. India, Cornmint Oil 50% Dementh. China, Cornmint Oil Rectified, Cubeb Oil, Cumin Seed Oil, Cypress Oil, Davana Oil, Dillseed Oil, Dillweed Oil, Eucalyptus Citriodora Brazil, Eucalyptus Citriodora Chinese, Eucalyptus Oil 70/75, Eucalyptus Oil 80/85, Eucalyptus Oil Terpenes, Eucalyptus Smithii Oil S.A., Evening Primrose, Fennel Oil, Fennel Oil Bitter, Fennel Oil Sweet, Fir Balsam Absolute, Fir Balsam Concrete, Fir Balsam Canadian, Fir Needle Oil Canadian, Fir Needle Oil Siberian, Fir Needle Oil Chinese, Galangal Root Oil, Galbanum Oil, Galbanum Resinoid, Garlic Oil Chinese, Garlic Oil Mexican, Genet Absolute, Geranium Oil Chinese, Geranium Oil Egyptian, Ginger Oil Chinese, Ginger Oil Fresh, Ginger Oil Indian, Grapefruit Oil 5×, Grapefruit Oil Pink C.P., Grapefruit Oil Washed, Grapefruit Oil White C.P., Grapefruit Terpenes, Grapeseed Oil, Gum Benzoin Siam Resin, Gurjon Balsam Oil, Guaiacwood Oil, Helichrysum Oil, Hemlock Oil, Ho Leaf Oil, Hop Oil, Horseradish Oil, Howood Oil 95%, Hyssop Oil, Jasmine Absolute, Jojoba Oil Purified White, Juniperberry Oil, Labdanum Gum Refined, Lanyana Oil, Laurel Leaf Oil, Lavandin Abrialis, Lavandin Grosso, Lavender Absolute Bulgarian, Lavender Oil 16%, Lavender Oil 40/42 French, Lavender Oil Bulgarian, Lavender Oil Spike, Lemon Essence Oil, Lemon Oil Argentina, Lemon Oil Brazil, Lemon Oil California 5×, Lemon Oil California Type, Lemon Oil Distilled, Lemon Oil Israeli, Lemon Oil Washed, Lemon Terpenes, Lemongrass Oil Guatemalan, Lemongrass Oil East Indian, Lemongrass Terpenes, Lemongrass Oil Terpeneless, Lime Essence Oil Phase, Lime Oil Mexican C.P., Types A & B, Lime Oil Distilled Mexican, Lime Oil Distilled Terpenes, Lime Oil Expressed Terpenes, Lime Oil Peru Distilled, Lime Oil Washed, Lime Sesqui Fractions, Lime Terpenes, Litsea Cubeba Oil, Litsea Terpenes, Lovage Leaf Oil, Lovage Root Oil, Mandarin Essence Oil Phase, Mandarin Oil Brazil, Mandarin Oil Green, Mandarin Oil Italian, Mandarin Oil Red Argentina, Mandarin Terpenes, Mandarin Petitgrain Terpeneless, Marjoram Oil Spanish, Marjoram Oil Sweet Egyptian, Massoia Bark Oil, Melissa Oil, Mentha Arvensis Oil, Mentha Citrata Oil, Mentha Piperita Oil, Milfoil Oil, Mimosa Absolute, Mousse de Arbre, Mousse de Pin Absolute, Mustard Oil Natural, Mustard Oil Synthetic, Myrrh Oil, Myrtle Oil, Neroli Oil, Nutmeg Oil, Nutmeg Oil Ceylon, Nutmeg Oil E.I., Nutmeg Terpenes, Oakmoss Absolute Green, Ocotea Cymbarum, Olibanum Oil, Olibanum Resin, Opoponax Oil, Onion Oil Egyptian, Onion Oil Mexican, Orange Juice Oil BJ N&A, Orange Essence Oil Phase, Orange Oil 5×, Orange Oil 10×, Orange Oil Bitter Ivory Coast Type, Orange Oil Bitter West Indian, Orange Oil Bitter Brazil, Orange Oil S.A., Orange Oil Midseason, Orange Oil Valencia, Orange Oil Terpeneless, Orange Terpenes, Organic Artemesia Oil, Organic Balsam Copaiba Oil, Organic Bois de Rose Oil, Organic Dillweed Oil, Organic Juniper Berry Oil, Organic Lavender Oil, Bulgarian, Organic Peppermint Oil, Organic Tea Tree Oil, Organic Wormwood Oil European, Organic Zdravetz Oil, Origanum Oil, Orris Root Concrete 8% Irone, Palmarosa Oil, Parsley Leaf Oil, Parsley Seed Oil, Patchouli Oil Indonesian, Patchouli Oil Light, Patchouli Oil Molecular Distilled, Pennyroyal Oil, Pepper Oil Black, Peppermint Oil Hotchkiss Type, Peppermint Oil Mitcham Type, Peppermint Oil Piperita Chinese, Peppermint Oil Piperita Indian, Peppermint Oil Piperita Redistilled, Peppermint Oil Piperita Williamette, Peppermint Oil Piperita Yakima, Peppermint Oil Terpenes, Perilla Oil, Petitgrain Oil S.A., Petitgrain Terpenes, Pimento Berry Oil, Pimento Leaf Oil, Pink Pepper Oil (Schinus Molle), Pinus Oil Pumilio, Pinus Oil Sylvestris, Rose Absolute Bulgarian, Rose Absolute Maroc, Rose Concrete Bulgarian, Rose Oil Bulgarian, Rose Oil Maroc, Rose Oil Turkish, Rose Water Concentrate, Rosemary Oil Maroc, Rosemary Oil Spanish, Rosemary Oil Tunisian, Rue Oil, Sage Oil 30%, Sage Oil 50%, Sage Oil Clary, Sage Oil Spanish, Sandalwood Oil Australian, Sandalwood Oil East Indian, Sandalwood Oil Indonesian, Sandalwood Oil Sri Lanka, Sassafras Oil, Savory Oil, Siamwood Oil, Spearmint Oil 65% Indian, Spearmint Oil Chinese 60%, Spearmint Oil Chinese 80%, Spearmint Oil Native, Spearmint Oil Terpeneless, Spearmint Terpenes, Spike Lavender Oil, Spikenard Oil, Spruce Absolute, Spruce Oil Canadian, Styrax Gum Honduras, Styrax Oil Honduras, Tagetes Oil Argentina, Tagetes Oil South African, Tagetes Oil Zimbabwe, Tangerine Oil 5×, Tangerine Oil 10×, Tangerine Oil CP China, Tangerine Oil CP Cravo, Tangerine Oil CP Florida, Tangerine Terpenes, Tarragon Oil, Tea Tree Oil, Thuja Oil, Thyme Oil Red, Thyme Oil White, Tolu Balsam, Tonka Bean Absolute, Perfume Grade, Treemoss Absolute, Tuberose Absolute, Valerian Root Oil, Vetiver Oil Indonesian, Vetiver Terpenes, Vetiver Oil Brazil, Violet Leaf Absolute, Wintergreen Oil Redistilled, Wormwood Oil American, Wormwood Oil European, Yarrow Oil (Milfoil), Ylang Oil #1, Ylang Oil #2, Ylang Oil #3, Ylang Oil Extra, and Zdravetz Oil.

### IX. Additional Repellents

As described herein, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, nootkatone, and VUAA-1 are chemical compounds with repellent properties.

The IUPAC name for methyl jasmonate is Methyl (1R,2R)-3-Oxo-2-(2Z)-2-pentenyl-cyclopentaneacetate. Methyl jasmonate is a volatile organic compound often used in plant defense and many diverse developmental pathways, such as seed germination, root growth, flowering, fruit ripening, and senescense (Cheong, Jong-Joo; Choi, Yang Do (July 2003). “Methyl jasmonate as a vital substance in plants”. Trends in Genetics. 19(7): 409-413.)

IR3535 is the trade name for ethyl butylacetylaminopropionate. IR3535 is an insect repellent.

2-undecanone is also known as methyl nonyl ketone or IBI-246. It a colorless oil typically used as an insect repellent.

Picaridin is also known as Icaridin. It IUPAC name is 1-(1-Methylpropoxycarbonyl)-2-(2-hydroxyethyl)piperidine. Picaridin is a near colorless and odorless insect repellent.

p-menthane-3,8-diol is also known aspara-menthane-3,8-diol, PMD, or menthoglycol. Its IUPAC name is 2-(2-Hydroxypropan-2-yl)-5-methylcyclohexan-1-ol.

The IUPAC name for alpha-terpineyl isovalerate is 2-(4-methylcyclohex-3-en-1-yl)propan-2-yl 3-methylbutanoate.

The IUPAC name for VUAA1 is 2-(4-Ethyl-5-(pyridin-3-yl)-4H-1,2,4-triazol-3-ylthio)-N-(4-ethylphenyl)acetamide. VUAA1 is a chemical compound that works by over activating an insect's olfactory senses, causing a repellent effect (Jones, P. L., Pask, G. M., Rinker, D. C., & Zwiebel, L. J. (2011). PNAS, 108(21), 8821-8825.).

The subject matter described herein includes the following embodiments:

1. A compound of Formula I

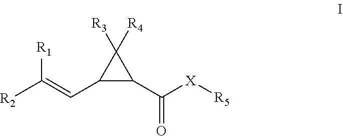

wherein R₁ and R₂ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R₁ and R₂ are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₃ and R₄ are each independently selected from hydrogen, haloalkyl, alkenyl, alkynyl, and C₁-C₆ alkyl;

R₅ is selected from the group consisting of linear or branched C₅-C₁₂ alkyl, haloalkyl, alkenyl, alkynyl, C₃-C₅ cycloalkyl, C₃-C₁₂ cycloalkylalkyl, C₆-C₁₂ heteroaryl, and C₃-C₁₂ heterocycloalkyl; and

X is selected from the group consisting of O, NH, and S.

2. The compound of embodiment 1, wherein R₃ and R₄ are each C₁-C₃ alkyl.
3. The compound of embodiment 1 or 2, wherein R₃ and R₄ are each methyl.
4. The compound of any one of embodiments 1-3, wherein R₁ and R₂ are each halo.
5. The compound of any one of embodiments 1-4, wherein R₁ and R₂ are each independently selected from chloro, bromo, iodo, and fluoro.
6. The compound of any one of embodiments 1-5, wherein R₁ and R₂ are each independently chloro.
7. The compound of any one of embodiments 1-6, wherein X is O.
8. The compound of any one of embodiments 1-7, wherein X in NH.
9. The compound of any one of embodiments 1-8, wherein R₅ is selected from the group consisting of linear C₅-C₁₂ alkyl, haloalkyl, and C₃-C₅ cycloalkyl.
10. The compound of any one of embodiments 1-9, wherein R₅ is selected from the group consisting of pentyl, trifluoroethyl, cyclobutyl, cyclopentyl, and cyclopropyl.
11. The compound of any one of embodiments 1-10, wherein said compound is selected from the group consisting of:

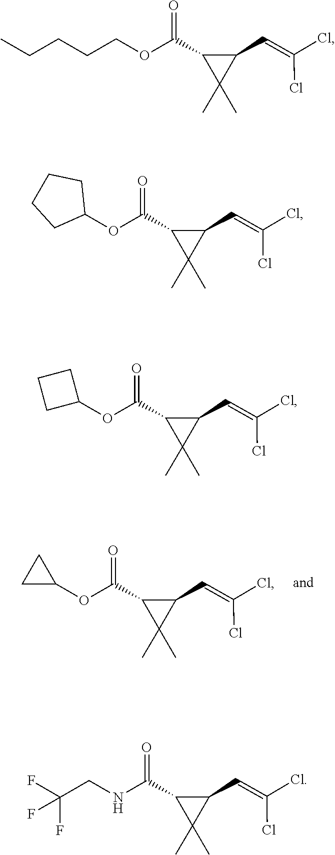 [SMILES: CCCCCOC(=O)[C@@H]1[C@@H](C=C(Cl)Cl)C1(C)C ; CC1(C)[C@H](C=C(Cl)Cl)[C@H]1C(=O)OC1CCCC1 ; CC1(C)[C@H](C=C(Cl)Cl)[C@H]1C(=O)OC1CCC1 ; CC1(C)[C@H](C=C(Cl)Cl)[C@H]1C(=O)OC1CC1 ; CC1(C)[C@H](C=C(Cl)Cl)[C@H]1C(=O)NCC(F)(F)F]

12. A composition comprising a compound of any one of embodiments 1-11 and a carrier.
13. A composition comprising about 0.01-99.99% of a compound of Formula II′ or a compound of Formula III′:

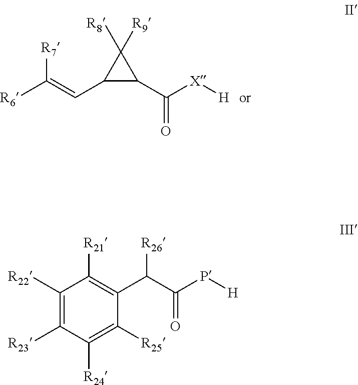

where R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl, and

about 99.99-0.01% of a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1,

wherein the composition is synergistic, in intimate admixture with a carrier.

14. The composition of embodiment 13, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.
15. The composition of embodiment 13 or 14, wherein said essential oil is citronella oil.
16. The composition of any one of embodiments 13-15, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.
17. The composition of any one of embodiments 13-16, wherein said composition comprises a compound of Formula II′, wherein X″ is O.
18. The composition of any one of embodiments 13-17, wherein R_{8′} and R_{9′} are each methyl.
19. The composition of any one of embodiments 13-18, wherein said compound of Formula II′ is

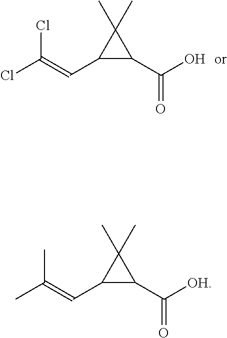

20. The composition of any one of embodiments 13-19, comprising about 0.01-99.99% of

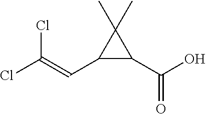

and about 99.99-0.01% transfluthrin.
21. The composition of any one of embodiments 13-20, comprising about 0.01-99.99%

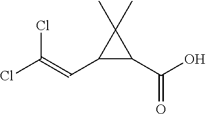

and about 99.99-0.01% citronella oil.
22. The composition of any one of embodiments 13-21, wherein said composition comprises about 0.01-99.99%% of a compound of Formula II′ and about 99.99-0.01% citronella oil.
23. A composition comprising about 0.01-99.99% of a compound of Formula IV, a compound of Formula V, or a compound of Formula VI:

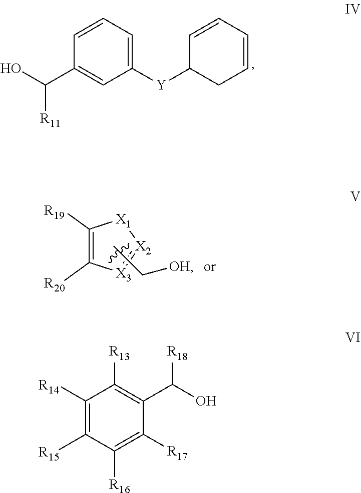

wherein Y is selected from the group consisting of O, NH, and S;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, and linear or branched C₁-C₆ alkyl; and

about 0.01-99.99% of a compound of Formula II′ or a compound of Formula III′:

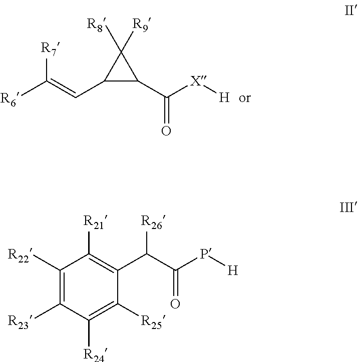

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl,

wherein the composition is synergistic, in intimate admixture with a carrier.

24. The composition of embodiment 23, wherein said composition comprises about 20-80% of a compound of Formula IV, a compound of Formula V, or a compound of Formula VI, and about 80-20% of a compound of Formula II′ or a compound of Formula III′.
25. The composition of embodiment 23 or 24, wherein said composition comprises about 40-60% of a compound of Formula IV, a compound of Formula V, or a compound of Formula VI, and about 60-40% of a compound of Formula II′ or a compound of Formula III′.
26. The composition of any one of embodiments 23-25, wherein said composition comprises about 0.01-99.99% a compound of Formula VI and about 99.99-0.01% a compound of Formula II′.
27. The composition of any one of embodiments 23-26, wherein said compound of Formula VI is

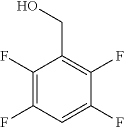 [SMILES: OCc1c(F)c(F)cc(F)c1F]

and said compound of Formula II′ is

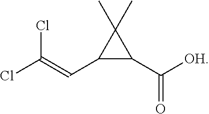

28. A composition comprising about 0.01-99.99% of a compound of Formula VII

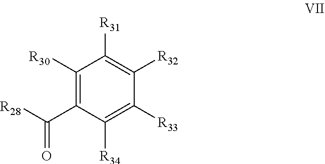

wherein R₂₈ is selected from the group consisting of hydrogen, haloalkyl, alkoxy, alkenyl, alkynyl, and C₁-C₆ linear or branched alkyl;

R₃₀, R₃₁, R₃₂, R₃₃, and R₃₄ are each independently selected from the group consisting of hydrogen, linear or branched C₁-C₆ alkyl, alkoxy, halo, cyano, haloalkyl, alkenyl, alkynyl, hydroxy, and

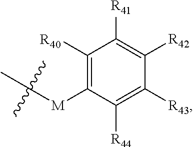

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy; and

about 99.99-0.01% of a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, VUAA-1, a compound of Formula II′, a compound of Formula III′, a compound of Formula IV, a compound of Formula V, and a compound of Formula VI

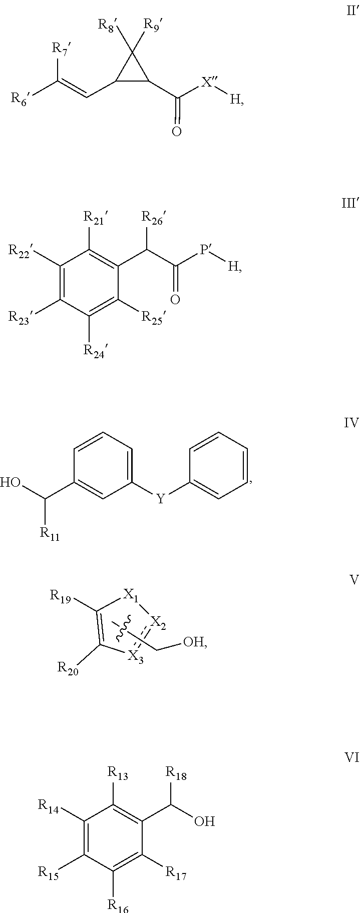

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl;

Y is selected from the group consisting of O, NH, and S;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, alkenyl, alkynyl, haloalkyl, and linear or branched C₁-C₆ alkyl,

wherein the composition is synergistic, in intimate admixture with a carrier.

29. The composition of embodiment 28, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.
30. The composition of embodiment 28 or 29, wherein said essential oil is citronella oil.
31. The composition of any one of embodiments 28-30, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.
32. The composition of any one of embodiments 28-31, wherein said composition comprises about 0.01-99.99% of a compound of Formula VII and about 99.99-0.01% of a compound selected from citronella oil and a compound of Formula II′.
33. The composition of any one of embodiments 28-32, wherein said composition comprises about 0.01-99.99% of a compound of Formula VII and about 99.99-0.01% of a compound selected from the group consisting of a compound of Formula IV, V, and VI.
34. The composition of any one of embodiments 28-33, wherein R₃₀, R₃₁, R₃₂, and R₃₄ are each hydrogen.
35. The composition of any one of embodiments 28-34, wherein R₃₃ is

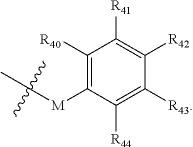

36. The compound of any one of embodiments 28-35, wherein R₄₀, R₄₁, R₄₂, R₄₃, and R₄₄ are each hydrogen.
37. The composition of any one of embodiments 28-36, wherein M is O.
38. The composition of any one of embodiments 28-37, wherein R₂₉ is hydrogen.
39. The composition of any one of embodiments 28-38, wherein said compound of Formula VII is

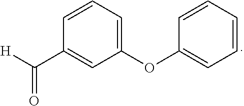 [SMILES: O=Cc1cccc(Oc2ccccc2)c1]

40. The composition of any one of embodiments 28-39, wherein said composition comprises from 0.01-99.99% of a compound of Formula VII having the structure

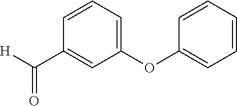 [SMILES: O=Cc1cccc(Oc2ccccc2)c1]

and from 99.99 to 0.01% of a compound of Formula II′ having the structure

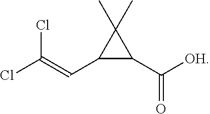

41. The composition of any one of embodiments 28-40, wherein said composition comprises from 0.01-99.99% of a compound of Formula VII having the structure

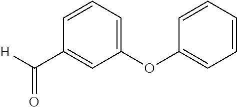 [SMILES: O=Cc1cccc(Oc2ccccc2)c1]

and from 99.99 to 0.01% of a compound of Formula II′ having the structure

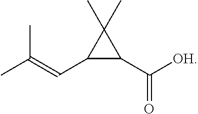

42. The composition of any one of embodiments 28-41, wherein said composition comprises from 0.01-99.99% of a compound of Formula VII having the structure

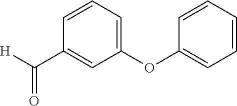 [SMILES: O=Cc1cccc(Oc2ccccc2)c1]

and from 99.99-0.1% of citronella oil.
43. The composition of any one of embodiments 28-42, wherein said composition comprises from 0.01-99.99% of a compound of Formula VII having the structure

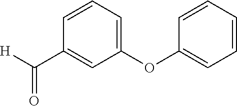 [SMILES: O=Cc1cccc(Oc2ccccc2)c1]

and from 99.99-0.01% of a compound of Formula VI having the structure

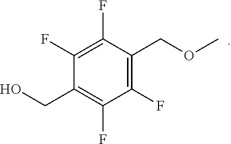

44. A composition comprising about 0.01-99.99% of a compound of Formula I′ and about 99.99-0.01% of a compound of Formula II′ or a compound of Formula III′

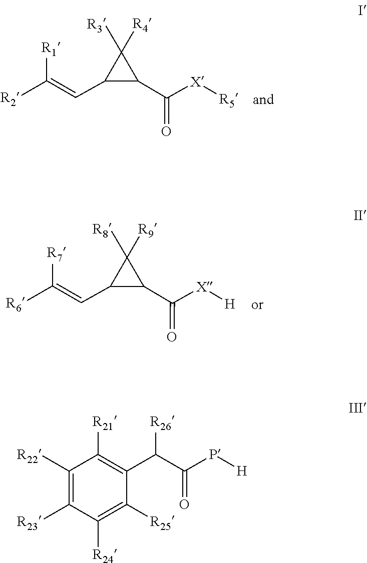

wherein R_{1′} and R_{2′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{1′} and R_{2′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{3′} and R_{4′} are each independently selected from hydrogen, haloalkyl, alkenyl, alkynyl, and C₁-C₆ alkyl;

R_{5′} is selected from the group consisting of linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X′ is selected from the group consisting of O, NH, and S;

R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{1′} and R_{2′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl, and

wherein the composition is synergistic, in intimate admixture with a carrier.

45. The composition of embodiment 44, wherein said composition comprises from 0.01-99.99% of a compound of Formula I′ and from 99.99 to 0.01% of a compound of Formula II′, wherein said compound of Formula II′ is

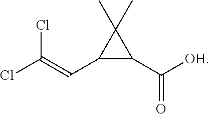

46. The composition of embodiment 44 or 45, wherein R_{3′} and R_{4′} are each methyl.
47. The composition of any one of embodiments 44-46, wherein X′ is O.
48. The composition of any one of embodiments 44-47, wherein R_{5′} is linear or branched C₁-C₆ alkyl.
49. The composition of any one of embodiments 44-48, wherein R_{5′} is C₃-C₆ cycloalkyl.
50. A method of repelling a species of insect from a locus to which said species of insect is normally attracted, comprising contacting on said locus a non-toxic effective insect repellent composition comprising a compound of Formula II′ or a compound of Formula III′ in an amount of about 0.01-99.99%:

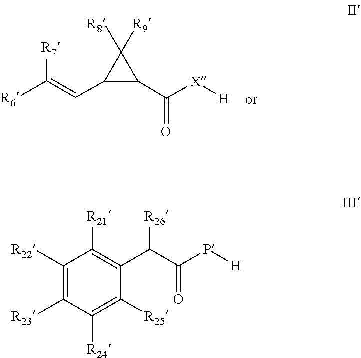

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl; and

a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, citronellol, citronellal, citral, oil of lemon eucalyptus, cinnamaldehyde, and VUAA-1, in an amount of about 99.99-0.01%,

wherein the composition is synergistic, in intimate admixture with a carrier.

51. The method of embodiment 50, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.
52. The method of embodiment 50 or 51, wherein said essential oil is citronella oil.
53. The method of any one of embodiments 50-52, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.
54. The method of any one of embodiments 50-53, wherein the composition contains about 0.0001 to 100000 parts per million of a compound of Formula II′ or a compound of Formula III′.
55. The method of any one of embodiments 50-54, wherein the composition is a solution, dust, granular formulation or emulsion.
56. The method of any one of embodiments 50-55, wherein the composition is a liquid solution or emulsion.
57. The method of any one of embodiments 50-56, wherein the composition contains about 0.0001 to 100000 parts per million of a compound of Formula II′ or a compound of Formula III′.
58. The method of any one of embodiments 50-57, wherein said species of insect is a fly, spider, butterfly, crab, mosquito, centipede, tick, millipede, or scorpion.
59. The method of any one of embodiments 50-58, wherein said insect is a mosquito.
60. The method of any one of embodiments 50-59, where the locus is an area, an environment, or the skin of an animal.
61. The method of any one of embodiments 50-60, wherein said composition comprises a compound of Formula II′.
62. The method of any one of embodiments 50-61, wherein X″ is O.
63. The method of any one of embodiments 50-62, wherein R_{8′} and R_{9′} are each C₁-C₆ alkyl.
64. The method of any one of embodiments 50-63, wherein R_{8′} and R_{9′} are each methyl.
65. The method of any one of embodiments 50-64, wherein R_{7′} and R_{6′} are each independently selected from halogen or C₁-C₆ alkyl.
66. The method of any one of embodiments 50-65, wherein said compound of Formula II′ is selected from

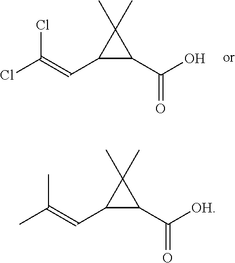

67. The method of any one of embodiments 50-66, wherein said composition comprises

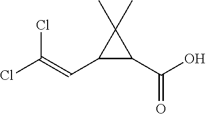

and transfluthrin.
68. The method of any one of embodiments 50-67, wherein said composition comprises

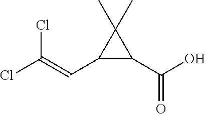

and citronella oil.
69. A method of repelling a species of insect from a locus to which said species of insect is normally attracted, comprising contacting on said locus a non-toxic effective insect repellent composition comprising a compound of Formula IV, a compound of Formula V, or a compound of Formula VI in an amount of about 0.01-99.99%:

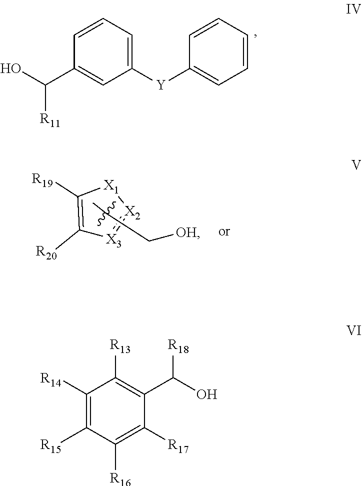

wherein Y is selected from the group consisting of O, NH, and S;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, haloalkyl, alkenyl, alkynyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, haloalkyl, alkenyl, alkynyl, C₁-C₆ alkyl, and C₁-C₃ alkyl-alkoxy;

R₁₈ is selected from the group consisting of hydrogen, haloalkyl, alkenyl, alkynyl, and linear or branched C₁-C₆ alkyl; and

a compound of Formula II′ or a compound of Formula III′ in an amount of about 99.99-0.01%:

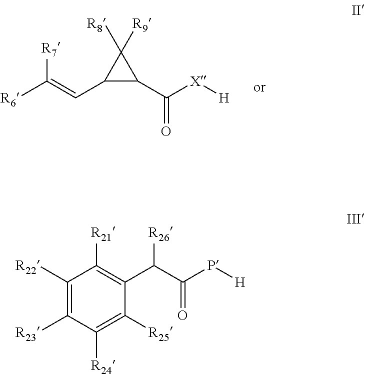

R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl,

wherein the composition is synergistic, in intimate admixture with a carrier.

70. The method of embodiment 69 wherein the composition contains about 0.0001 to 100000 parts per million of a compound of Formula VI.
71. The method of embodiment 69 or 70, wherein R₁₈ is hydrogen.
72. The method of any one of embodiments 69-71, wherein R₁₃, R₁₄, R₁₆, and R₁₇ are each halo.
73. The method of any one of embodiments 69-72, wherein R₁₃, R₁₄, R₁₆, and R₁₇ are each fluoro.
74. The method of any one of embodiments 69-73, wherein R₁₆ is selected from hydrogen or C₁-C₃ alkyl-alkoxy.
75. The method of any one of embodiments 69-74, wherein said compound of Formula VI is

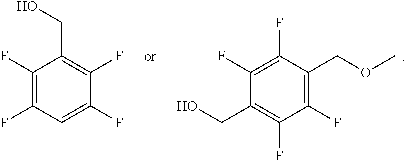

76. The method of any one of embodiments 69-75, wherein said composition comprises a compound of Formula VI and a compound of Formula II′.
77. The method of any one of embodiments 69-76, wherein X″ is O.
78. The method of any one of embodiments 69-77, wherein R_{8′} and R_{9′} are each methyl.
79. The method of any one of embodiments 69-78, wherein R_{7′} and R_{6′} are each independently selected from halogen or C₁-C₆ alkyl.
80. The method of any one of embodiments 69-79, wherein said compound of Formula VI is

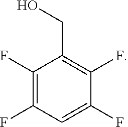 [SMILES: OCc1c(F)c(F)cc(F)c1F]

81. The method of any one of embodiments 69-80, wherein said composition comprises a compound of Formula VI of

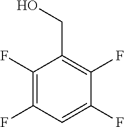 [SMILES: OCc1c(F)c(F)cc(F)c1F]

and a compound of Formula II′ of

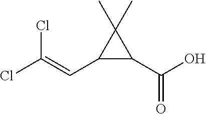

82. The method of any one of embodiments 69-81, wherein the composition is a solution, dust, granular formulation or emulsion.
83. The method of any one of embodiments 69-82, wherein the composition is a liquid solution or emulsion.
84. The method of any one of embodiments 69-83, wherein the composition contains about 0.0001 to 100000 parts per million of a compound of Formula IV, Formula V, or Formula VI.
85. The method of any one of embodiments 69-84, wherein said species of insect is a fly, spider, butterfly, crab, mosquito, centipede, tick, millipede, or scorpion.
86. The method of any one of embodiments 69-85, wherein said insect is a mosquito.
87. The method of any one of embodiments 69-86, where the locus is an area, an environment, or the skin of an animal.
88. A method of repelling a species of insect from a locus to which said species of insect is normally attracted, comprising contacting on said locus a non-toxic effective insect repellent composition comprising a compound of Formula VII in an amount of about 0.01-99.99%

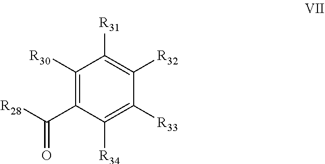

wherein R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, haloalkyl, alkenyl, alkynyl, linear or branched C₁-C₆ alkyl, alkoxy, halo, cyano, hydroxy, and

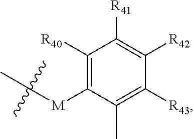

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy; and

a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, VUAA-1, a compound of Formula II′, a compound of Formula III′, a compound of Formula IV, a compound of Formula V, and a compound of Formula VI in an amount of about 99.99-0.01%

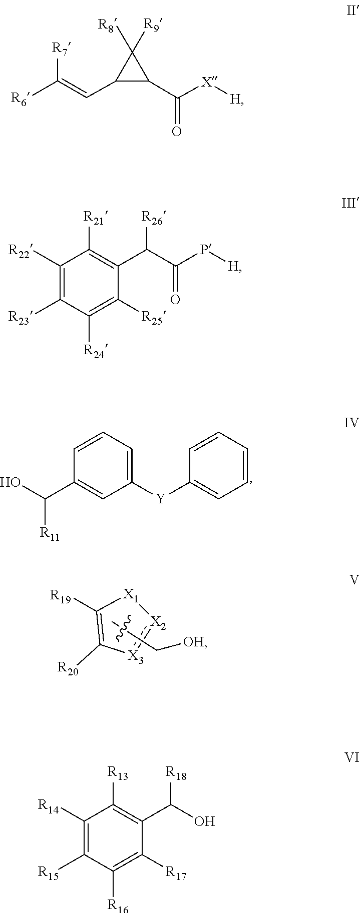

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl;

Y is selected from the group consisting of O, NH, and S;

R₁₁ is selected from the group consisting of hydrogen, alkenyl, alkynyl, C₁-C₆ alkyl, CN, halo, and haloalkyl;

R₁₉ and R₂₀ are each independently selected from the group consisting of hydrogen, C₁-C₆ alkyl, cycloalkylalkyl, arylalkyl, haloalkyl, alkenyl, alkynyl, cycloalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, haloalkyl, alkenyl, alkynyl, halo, C₁-C₆ alkyl, and C₁-C₃ alkyl-alkoxy;

R₁₈ is selected from the group consisting of hydrogen, haloalkyl, alkenyl, alkynyl, and linear or branched C₁-C₆ alkyl,

wherein the composition is synergistic, in intimate admixture with a carrier.

89. The method of embodiment 88 wherein the composition contains about 0.0001 to 100000 parts per million of a compound of Formula VII.
90. The method of embodiment 88 or 89, wherein R₃₀, R₃₁, R₃₂, and R₃₄ are each hydrogen.
91. The method of any one of embodiments 88-90, wherein R₃₃ is

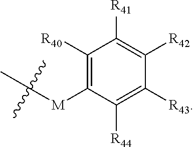

92. The method of any one of embodiments 88-91, wherein R₄₀, R₄₁, R₄₂, R₄₃, and R₄₄ are each hydrogen.
93. The method of any one of embodiments 88-92, wherein M is O.
94. The method of any one of embodiments 88-93, wherein R₂₈ is hydrogen.
95. The method of any one of embodiments 88-94, wherein said compound of Formula VII is

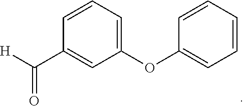 [SMILES: O=Cc1cccc(Oc2ccccc2)c1]

96. The method of any one of embodiments 88-95, wherein said composition comprises

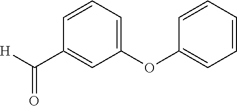 [SMILES: O=Cc1cccc(Oc2ccccc2)c1]

and citronella oil.
97. The method of any one of embodiments 88-96, wherein said composition comprises

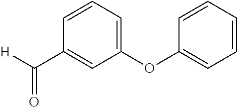 [SMILES: O=Cc1cccc(Oc2ccccc2)c1]

and a compound of Formula VI.
98. The method of any one of embodiments 88-97, wherein said composition comprises

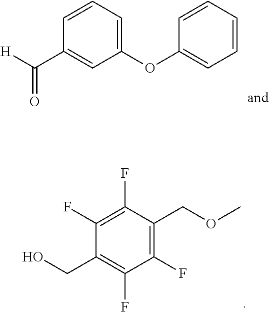

99. The method of any one of embodiments 88-98, wherein said composition comprises

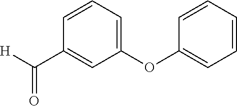 [SMILES: O=Cc1cccc(Oc2ccccc2)c1]

and a compound of Formula II′.
100. The method of any one of embodiments 88-99, wherein said composition comprises

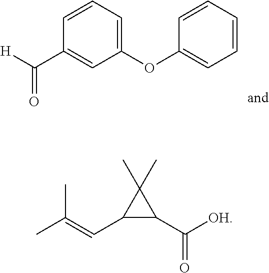

101. The method of any one of embodiments 88-100, wherein said composition comprises

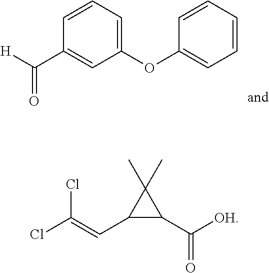

102. The method of any one of embodiments 88-101, wherein the composition is a solution, dust, granular formulation or emulsion.
103. The method of any one of embodiments 88-102, wherein the composition is a liquid solution or emulsion.
104. The method of any one of embodiments 88-103, wherein the composition contains about 0.0001 to 100000 parts per million of a compound of Formula VII.
105. The method of any one of embodiments 88-104, wherein said species of insect is a fly, spider, butterfly, crab, mosquito, centipede, tick, millipede, or scorpion.
106. The method of any one of embodiments 88-105, wherein said insect is a mosquito.
107. The method of any one of embodiments 88-106, where the locus is an area, an environment, or the skin of an animal.
108. A method of repelling a species of insect from a locus to which said species of insect is normally attracted, comprising contacting on said locus a non-toxic effective insect repellent composition comprising a compound of Formula I′ in an amount of about 0.01-99.99%

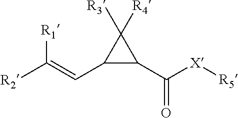

I′ and a compound of Formula II′ or a compound of Formula III′ in an amount of about 99.99-0.01%

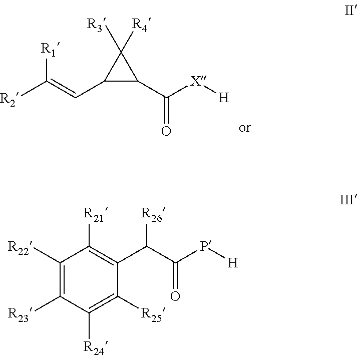

wherein R_{1′} and R_{2′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{3′} and R_{4′} are each independently selected from hydrogen, haloalkyl, alkenyl, alkynyl, and C₁-C₆ alkyl;

R_{5′} is selected from the group consisting of linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X′ is selected from the group consisting of O, NH, and S;

R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl,

wherein the composition is synergistic, in intimate admixture with a carrier.

109. The method of embodiment 108, wherein the composition contains about 0.0001 to 100000 parts per million of a compound of Formula I′.
110. The method of embodiment 108 or 109, wherein R_{3′} and R_{4′} are each methyl.
111. The method of any one of embodiments 108-110, wherein X′ is O.
112. The method of any one of embodiments 108-111, wherein R_{5′} is linear or branched C₁-C₆ alkyl.
113. The method of any one of embodiments 108-112, wherein the composition is a solution, dust, granular formulation, or emulsion.
114. The method of any one of embodiments 108-113, wherein the composition is a liquid solution or emulsion.
115. The method of any one of embodiments 108-114, wherein the composition contains about 0.0001 to 100000 parts per million of the compound of Formula I′.
116. The method of any one of embodiments 108-115, wherein the composition contains a compound of Formula I′ and a compound of Formula II′.
117. The method of any one of embodiments 108-116, wherein X″ is O.
118. The method of any one of embodiments 108-117, wherein R_{6′} and R_{7′} are each independently selected from halo or C₁-C₆ alkyl.
119. The method of any one of embodiments 108-118, wherein R_{8′} and R_{9′} are each methyl.
120. The method of any one of embodiments 108-119, wherein the compound of Formula II″ is

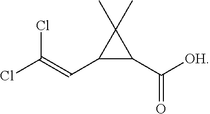

121. The method of any one of embodiments 108-120, wherein the composition comprises a compound of Formula I′ and

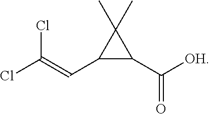

122. The method of any one of embodiments 108-121, wherein the compound of Formula I′ is

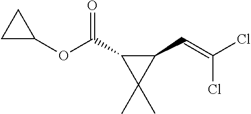

123. The method of any one of embodiments 108-122, wherein said species of insect is a fly, spider, butterfly, crab, mosquito, centipede, tick, millipede, or scorpion.
124. The method of any one of embodiments 108-123, wherein said insect is a mosquito.
125. The method of any one of embodiments 108-124, where the locus is an area, an environment, or the skin of an animal.
126. A method for controlling one or more insects, comprising contacting the insects with a synergistic composition comprising a compound of Formula II′ or a compound of Formula III′ in an amount of about 0.01-99.99%:

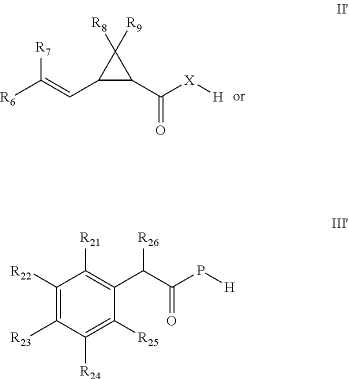

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl; and

a pyrethroid in an amount of about 99.99 to 0.01%,

wherein the synergistic composition produces, when the insects are brought into contact with the synergistic composition, a combined toxicant effect greater than the sum of the separate toxicant effects from the compound of Formula II′ or Formula III′ and the pyrethroid, at comparable concentrations.

127. The method of embodiment 126, wherein said toxicant effect is the vapor toxicity of the synergistic composition.
128. The method of embodiment 126 or 127, wherein the synergistic composition contains about 0.0001 to 100000 parts per million of a compound of Formula II′ or Formula III′.
129. The method of any one of embodiments 126-128, wherein said synergistic composition comprises a compound of Formula II′ and transfluthrin.
130. The method of any one of embodiments 126-129, wherein X″ is O.
131. The method of any one of embodiments 126-130, wherein R_{6′} and R_{7′} are selected from halo or C₁-C₆ alkyl.
132. The method of any one of embodiments 126-131, wherein R_{8′} and R_{9′} are each independently C₁-C₃ alkyl.
133. The method of any one of embodiments 126-132, wherein R_{8′} and R_{9′} are each methyl.
134. The method of any one of embodiments 126-133, wherein said compound of Formula II is

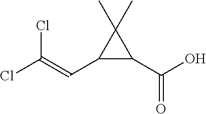

135. The method of any one of embodiments 126-134, wherein said composition comprises

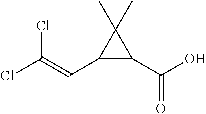

and transfluthrin.
136. The method of any one of embodiments 126-135, wherein the synergistic composition is a solution, dust, granular formulation or emulsion.
137. The method of any one of embodiments 126-136, wherein the synergistic composition is a liquid solution or emulsion.
138. The method of any one of embodiments 126-137, wherein the synergistic composition contains about 0.0001 to 100000 parts per million of a compound of Formula II′.
139. The method of any one of embodiments 126-138, wherein said insects are flies, spiders, butterflies, crabs, mosquitos, centipedes, ticks, millipedes, or scorpions.
140. The method of any one of embodiments 126-139, wherein said insects are mosquitos.
141. A method of repelling a species of insect from a locus to which said species of insect is normally attracted, comprising contacting on said locus a non-toxic effective insect repellent composition comprising a compound of Formula II, Formula III, Formula IV, Formula V, Formula VI, or Formula VII

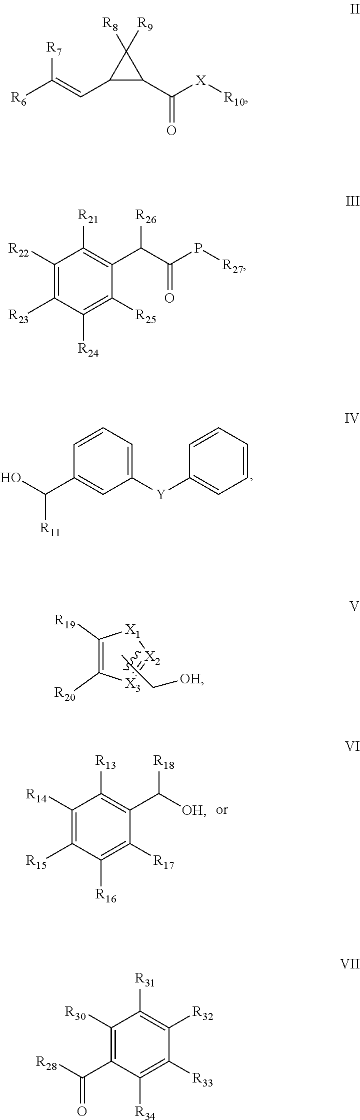

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₈ and R₉ are each independently selected from hydrogen, haloalkyl, alkenyl, alkynyl, and C₁-C₆ alkyl;

R₁₀ is selected from the group consisting of hydrogen, haloalkyl, alkenyl, alkynyl, linear or branched C₁-C₁₂ alkyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, alkoxy, halo, C₁-C₆ alkyl, haloalkyl, alkenyl, alkynyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl;

R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, haloalkyl, alkenyl, alkynyl, C₁-C₆ alkyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

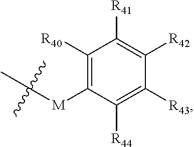

wherein M is selected from the group consisting of O, NH, and S; and

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy.

142. The method of embodiment 141, wherein the composition is a solution, dust, granular formulation or emulsion.
143. The method of embodiment 141 or 142, wherein the composition is a liquid solution or emulsion.
144. The method of any one of embodiments 141-143, wherein said species of insect is a fly, spider, butterfly, crab, mosquito, centipede, tick, millipede, or scorpion.
145. The method of any one of embodiments 141-144, wherein said insect is a mosquito.
146. The method of any one of embodiments 141-145, wherein the mosquito is Aedes aegypti.
147. The method of any one of embodiments 141-146, where the locus is an area, an environment, or the skin of an animal.
148. The method of any one of embodiments 141-147, wherein said compound is of Formula II, wherein X is O and R₁₀ is hydrogen.
149. The method of any one of embodiments 141-148, wherein R₈ and R₉ are each methyl.
150. The method of any one of embodiments 141-149, wherein said compound is selected from the group consisting of

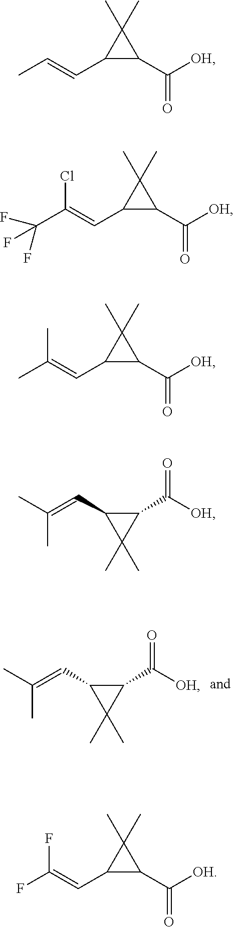

151. The method of any one of embodiments 141-150, wherein said compound is of Formula III.
152. The method of any one of embodiments 141-151, wherein P is O and R₂₇ is H.
153. The method of any one of embodiments 141-152, wherein R₂₃ is halo and R₂₁, R₂₂, R₂₄, and R₂₅ are each hydrogen.
154. The method of any one of embodiments 141-153, wherein R₂₃ is chloro.
155. The method of any one of embodiments 141-154, wherein said compound is

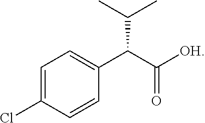

156. The method of any one of embodiments 141-155, wherein said compound is of Formula VI.
157. The method of any one of embodiments 141-156, wherein R₁₃, R₁₄, R₁₆, and R₁₇ are each halo.
158. The method of any one of embodiments 141-157, wherein R₁₃, R₁₄, R₁₆, and R₁₇ are each fluoro.
159. The method of any one of embodiments 141-158, wherein R₁₅ is hydrogen.
160. The method of any one of embodiments 141-159, wherein R₁₈ is hydrogen.
161. The method of any one of embodiments 141-160, wherein the compound of Formula VI is

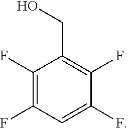 [SMILES: OCc1c(F)c(F)cc(F)c1F]

162. The method of any one of embodiments 141-161, wherein the compound is of Formula IV.
163. The method of any one of embodiments 141-162, wherein Y is O.
164. The method of any one of embodiments 141-163, wherein R₁₁ is CN.
165. The method of any one of embodiments 141-164, wherein the compound of Formula IV is

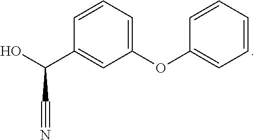 [SMILES: N#C[C@@H](O)c1cccc(Oc2ccccc2)c1]

166. The method of any one of embodiments 141-165, wherein the compound is of Formula VII.
167. The method of any one of embodiments 141-166, wherein R₃₀, R₃₁, R₃₂, and R₃₄ are each hydrogen.
168. The method of any one of embodiments 141-167, wherein R₃₃ is

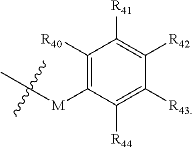

169. The method of any one of embodiments 141-168, wherein R₄₀, R₄₁, R₄₂, R₄₃, and R₄₄ are each hydrogen.
170. The method of any one of embodiments 141-169, wherein M is O.
171. The method of any one of embodiments 141-170, wherein R₂₈ is H.
172. The method of any one of embodiments 141-171, wherein the compound of Formula VII is

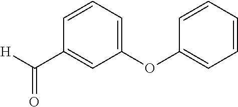 [SMILES: O=Cc1cccc(Oc2ccccc2)c1]

173. The method of any one of embodiments 141-172, wherein the compound is of Formula II, wherein X is O and R₁₀ is selected from C₃-C₆ cycloalkyl and linear or branched C₁-C₆ alkyl.
174. A method for controlling one or more insects, comprising exposing said one or more insects to a synergistic composition comprising a compound of Formula II′ or a compound of Formula III′, and a pyrethroid;

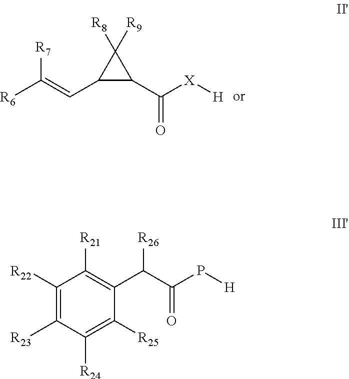

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl;

wherein the synergistic composition produces, when said one or more insects are exposed to the synergistic composition, a combined toxicant effect greater than the sum of the separate toxicant effects from the compound of Formula II′ or Formula III′ and the pyrethroid, at comparable concentrations.

175. The method of embodiment 174, wherein said toxicant effect is the vapor toxicity of the synergistic composition.
176. The method of embodiment 174 or 175, wherein the synergistic composition contains about 0.0001 to 100000 parts per million of a compound of Formula II′ or Formula III′.
177. The method of any one of embodiments 174-176, wherein said synergistic composition comprises a compound of Formula II′ and transfluthrin.
178. The method of any one of embodiments 174-177, wherein X″ is O.
179. The method of any one of embodiments 174-178, wherein R_{6′} and R_{7′} are selected from halo or C₁-C₆ alkyl.
180. The method of any one of embodiments 174-179, wherein R_{8′} and R_{9′} are each independently C₁-C₃ alkyl.
181. The method of any one of embodiments 174-180, wherein R_{8′} and R_{9′} are each methyl.
182. The method of any one of embodiments 174-181, wherein said compound of Formula II is

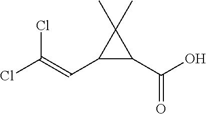

183. The method of any one of embodiments 174-1181, wherein said composition comprises

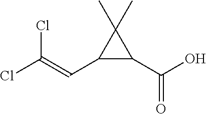

and transfluthrin.
184. The method of any one of embodiments 174-183, wherein the synergistic composition is a solution, dust, granular formulation or emulsion.
185. The method of any one of embodiments 174-183, wherein the synergistic composition is a liquid solution or emulsion.
186. The method of any one of embodiments 174-185, wherein the synergistic composition contains about 0.0001 to 100000 parts per million of a compound of Formula II′.
187. The method of any one of embodiments 174-186, wherein said one or more insects are flies, spiders, butterflies, crabs, mosquitos, centipedes, ticks, millipedes, or scorpions.
188. The method of any one of embodiments 174-186, wherein said one or more insects are mosquitos.
189. A composition comprising an essential oil, and a second compound selected from a pyrethroid, a compound of Formula II, Formula III, Formula IV, Formula V, Formula VI, or Formula VII, wherein the composition is synergistic, in intimate admixture with a carrier; or, comprising about 0.01-99.99% of an essential oil; and about 99.99-0.01% of a second compound selected from the group consisting of a pyrethroid, a compound of Formula II, Formula III, Formula IV, Formula V, Formula VI, or Formula VII

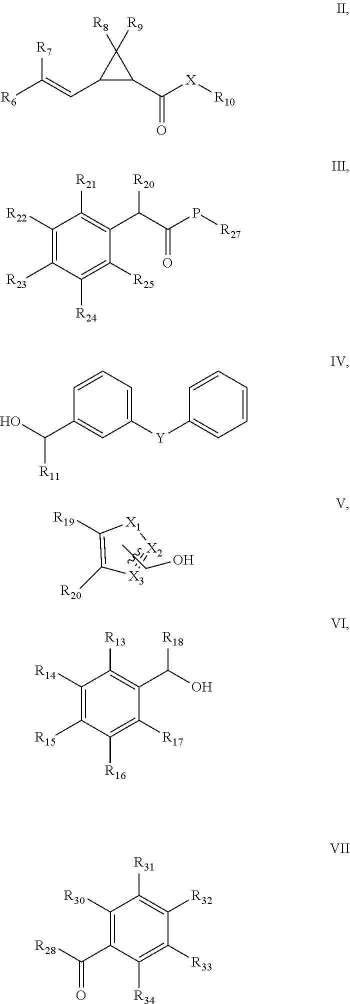

wherein R₆ and R₇ are each independently selected from the group consisting of hydrogen, halo, haloalkyl, C₆-C₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₈ and R₉ are each independently selected from hydrogen haloalkyl, alkenyl, alkynyl, and C₁-C₆ alkyl;

R₁₀ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, alkoxy, halo, haloalkyl, alkenyl, alkynyl, C₁-C₆ alkyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl;

R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, haloalkyl, alkenyl, alkynyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

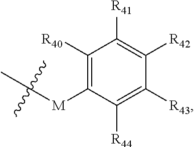

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy,

wherein the composition is synergistic, in intimate admixture with a carrier.

190. The composition of embodiment 189, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.
191. The composition of embodiment 189 or 190, wherein said essential oil is citronella oil.
192. The composition of embodiment 189, wherein said second compound is a pyrethroid.
193. The composition of any one of embodiments 189-192, wherein said pyrethroid is selected from the group consisting of permethrin, tetramethrin, metofluthrin, bifenthrin, kadethrin, allethrin, cyfluthrin, deltamethrin, empenthrin, cypermethrin, cyphenothrin, esfenvalerate, fenvalerate, flumethrin, tefluthrin, phenothrin, and transfluthrin.
194. The composition of any one of embodiments 189-193, wherein said pyrethroid is metofluthrin.
195. The composition of embodiment 189, wherein said composition comprises a pyrethroid, wherein said pyrethroid is metofluthrin and said essential oil is selected from the group consisting of citronella oil, Canadian balsam, galbanum oil, ginger root oil, dill seed oil, and cypress oil.
196. The composition of any one of embodiments 189-195, wherein said composition comprises metofluthrin having a concentration of about 0.1 μg/cm², and said essential oil having a concentration selected from the group consisting of 100 μg/cm², 30 μg/cm², and 20 μg/cm².
197. A method of repelling a species of insect from a locus to which said species of insect is normally attracted, comprising contacting on said locus a non-toxic effective insect repellent of an essential oil in an amount of about 0.01-99.99%, and a second compound selected from the group consisting of a pyrethroid, a compound of Formula II, Formula III, Formula IV, Formula V, Formula VI, and Formula VII, wherein said second compound is present in an amount from 99.99-0.01%,

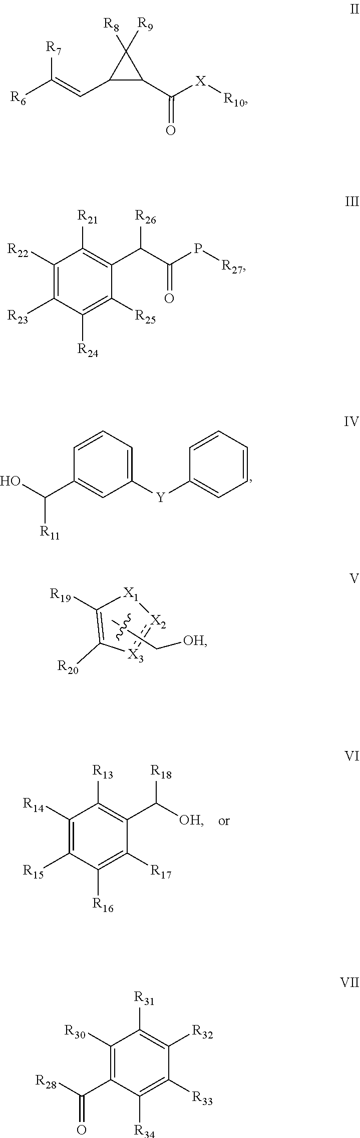

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₈ and R₉ are each independently selected from hydrogen haloalkyl, alkenyl, alkynyl, and C₁-C₆ alkyl;

R₁₀ is selected from the group consisting of hydrogen, linear or branched C₁-C₁₂ alkyl, C₃-C₁₂ cycloalkyl, haloalkyl, alkenyl, alkynyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, alkoxy, halo, C₁-C₆ alkyl, haloalkyl, alkenyl, alkynyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl;

R₂₇ is selected from the group consisting of hydrogen, linear or branched C₁-C₁₂ alkyl, C₃-C₁₂ cycloalkyl, haloalkyl, alkenyl, alkynyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and

wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

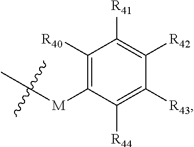

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy, and

wherein the composition is synergistic, in intimate admixture with a carrier.

198. The method of embodiment 197, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.
199. The method of embodiment 197 or 198, wherein said essential oil is citronella oil.
200. The method of any one of embodiments 197-199, wherein said second compound is a pyrethroid.
201. The method of any one of embodiments 197-200, wherein said pyrethroid is selected from the group consisting of permethrin, empenthrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.
202. The method of any one of embodiments 197-201, wherein said pyrethroid is metofluthrin.
203. The method of any one of embodiments 197-202, wherein said composition comprises a pyrethroid, wherein said pyrethroid is metofluthrin, and said essential oil is selected from the group consisting of citronella oil, Canadian balsam, galbanum oil, ginger root oil, dill seed oil, and cypress oil.
204. The method of any one of embodiments 197-203, wherein said composition comprises metofluthrin having a concentration of about 0.1 μg/cm², and said essential oil having a concentration selected from the group consisting of 100 μg/cm², 30 μg/cm², and 20 μg/cm².
205. The method of any one of embodiments 50-188 or 197-204, wherein said species of insect is not physically contacted with said locus.
206. A method of providing a composition of any one of embodiments 1-205 in an insect repellent medium from which a vaporized composition of any one of embodiments 1-205 can be dispersed.
208. The method of any one of embodiments 50-188 or 197-205, wherein the composition is formulated for use in a vaporizer, evaporator, fan, heat, candle, or wicked apparatus.
209. The method of any one of embodiments 197-204 or the composition comprising any one of embodiments 189-196, wherein said essential oil is citronella oil and said second compound is metofluthrin.
210. The method of any one of embodiments 197-204 or the composition comprising any one of embodiments 189-196, wherein said essential oil is citronella oil and said second compound is empenthrin.
211. A method of repelling an arthropod from an object or locus, comprising contacting on said object or locus a composition comprising a compound of Formula II′ or a compound of Formula III′:

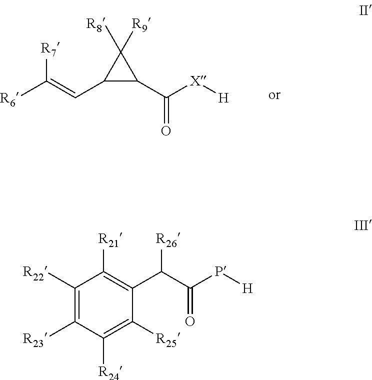

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl; and

a compound selected from the group consisting of an essential oil, a pyrethroid, methyl jasmonate, IR3535, 2-undecanone, picaridin, benzaldehyde, p-menthane-3,8-diol, alpha-terpineyl isovalerate, nootkatone, DEET, benzyl benzoate, ethyl hexanediol, diethyl phthalate, diethyl carbate, geraniol, citronellol, citronellal, citral, oil of lemon eucalyptus, cinnamaldehyde, and VUAA-1,

wherein the composition is synergistic, in admixture with a carrier.

212. The method of embodiment 211, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.
213. The method of embodiment 211 or 212, wherein said essential oil is citronella oil.
214. The method of embodiment 211, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, empenthrin, and transfluthrin.
215. The method of any one of embodiments 211-214, wherein the composition contains about 0.0001 to 100000 parts per million of a compound of Formula II′ or a compound of Formula III′.
216. The method of any one of embodiments 211-215, wherein the composition is a solution, dust, granular formulation or emulsion.
217. The method of any one of embodiments 211-216, wherein the composition is a liquid solution or emulsion.
218. The method of any one of embodiments 211-217, wherein the composition contains about 0.0001 to 100000 parts per million of a compound of Formula II′ or a compound of Formula III′.
219. The method of any one of embodiments 211-218, wherein said arthropod is a fly, spider, butterfly, crab, mosquito, centipede, tick, millipede, or scorpion.
220. The method of embodiment 219, wherein said arthropod is a mosquito.
221. The method of any one of embodiments 211-221, where the object or locus is an area, an environment, or the skin of an animal.
222. The method of any one of embodiments 211-221, wherein said composition comprises a compound of Formula II′.
223. The method of any one of embodiments 211-222, wherein X″ is O.
224. The method of any one of embodiments 211-223, wherein R_{8′} and R_{9′} are each C₁-C₆ alkyl.
225. The method of any one of embodiments 211-224, wherein R_{8′} and R_{9′} are each methyl.
226. The method of any one of embodiments 211-225, wherein R_{7′} and R_{6′} are each independently selected from halogen or C₁-C₆ alkyl.
227. The method of any one of embodiments 211-226, wherein said compound of Formula II′ is selected from

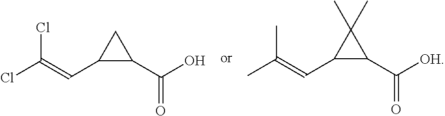

228. The method of any one of embodiments 211-227, wherein said composition comprises

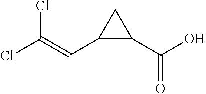

and transfluthrin.
229. The method of any one of embodiments 211-228, wherein said composition comprises

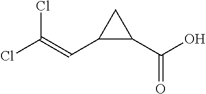

and citronella oil.
230. A method of repelling an arthropod from an object or locus, comprising contacting said object or locus with a composition comprising a compound of Formula IV, a compound of Formula V, or a compound of Formula VI

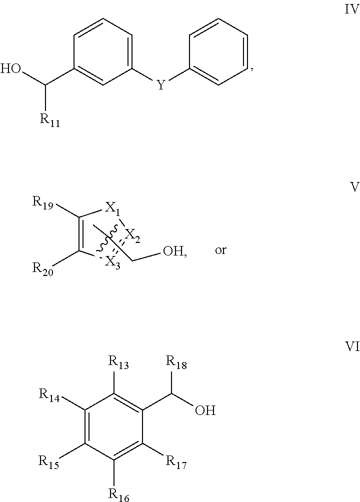

wherein Y is selected from the group consisting of O, NH, and S;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, haloalkyl, alkenyl, alkynyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and
wherein at least one of X₁, X₂, and X₃ is substituted with —COH;

R₁₃, R₁₄, R₁₅, R₁₆, and R₁₇ are each independently selected from the group consisting of hydrogen, alkoxy, halo, haloalkyl, alkenyl, alkynyl, C₁-C₆ alkyl, and C₁-C₃ alkyl-alkoxy;

R₁₈ is selected from the group consisting of hydrogen, haloalkyl, alkenyl, alkynyl, and linear or branched C₁-C₆ alkyl; and

a compound of Formula II′ or a compound of Formula III′

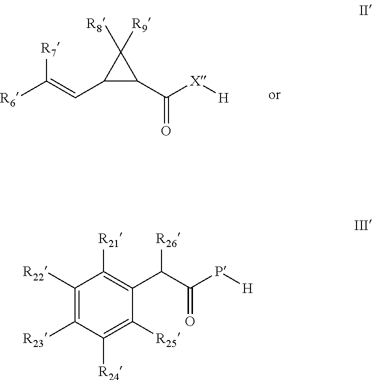

R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl,

wherein the composition is synergistic, in admixture with a carrier.

231. The method of embodiment 230, wherein the composition contains about 0.0001 to 100000 parts per million of a compound of Formula VI.
232. The method of embodiment 230 or 231, wherein R₁₈ is hydrogen.
233. The method of any one of embodiments 230-232, wherein R₁₃, R₁₄, R₁₆, and R₁₇ are each halo.
234. The method of any one of embodiments 230-233, wherein R₁₃, R₁₄, R₁₆, and R₁₇ are each fluoro.
235. The method of any one of embodiments 230-234, wherein R₁₆ is selected from hydrogen or C₁-C₃ alkyl-alkoxy.
236. The method of any one of embodiments 230-235, wherein said compound of Formula VI is

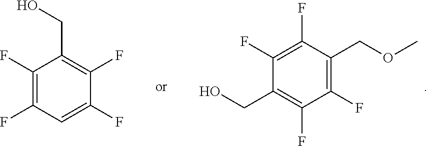

237. The method of any one of embodiments 230-236, wherein said composition comprises a compound of Formula VI and a compound of Formula II′.
238. The method of any one of embodiments 230-237, wherein X″ is O.
239. The method of any one of embodiments 230-238, wherein R_{8′} and R_{9′} are each methyl.
240. The method of any one of embodiments 230-239, wherein R_{7′} and R_{6′} are each independently selected from halogen or C₁-C₆ alkyl.
241. The method of any one of embodiments 230-240, wherein said compound of Formula VI is

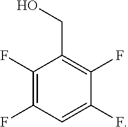 [SMILES: OCc1c(F)c(F)cc(F)c1F]

242. The method of any one of embodiments 230-241, wherein said composition comprises a compound of Formula VI of

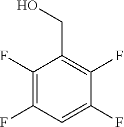 [SMILES: OCc1c(F)c(F)cc(F)c1F]

and a compound of Formula II′ of

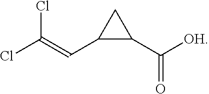

243. The method of any one of embodiments 230-243, wherein the composition is a solution, dust, granular formulation or emulsion.
244. The method of any one of embodiments 230-243, wherein the composition is a liquid solution or emulsion.
245. The method of any one of embodiments 230-244, wherein the composition contains about 0.0001 to 100000 parts per million of a compound of Formula IV, Formula V, or Formula VI.
246. The method of any one of embodiments 230-245, wherein said arthropod is a fly, spider, butterfly, crab, mosquito, centipede, tick, millipede, or scorpion.
247. The method of any one of embodiments 230-247, wherein said arthropod is a mosquito.
248. The method of any one of embodiments 230-247, wherein the object or locus is an area, an environment, or the skin of an animal.
249. A method of repelling an arthropod from an object or locus, comprising contacting said object or locus with a composition comprising a compound of Formula VII

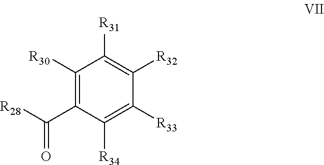

wherein R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, haloalkyl, alkenyl, alkynyl, linear or branched C₁-C₆ alkyl, alkoxy, halo, cyano, hydroxy, and

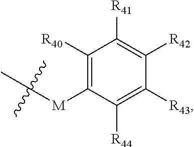

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy; and

a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, VUAA-1, nootkatone, a compound of Formula II′, a compound of Formula III′, a compound of Formula IV, a compound of Formula V, and a compound of Formula VI

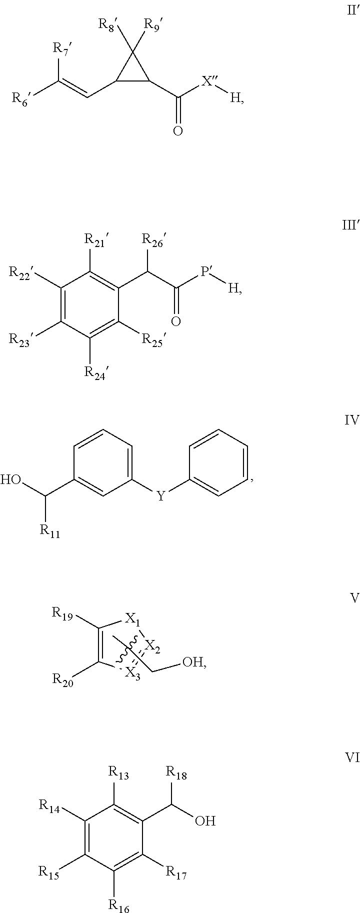

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl;

Y is selected from the group consisting of O, NH, and S;

R₁₁ is selected from the group consisting of hydrogen, alkenyl, alkynyl, C₁-C₆ alkyl, CN, halo, and haloalkyl;

R₁₉ and R₂₀ are each independently selected from the group consisting of hydrogen, C₁-C₆ alkyl, cycloalkylalkyl, arylalkyl, haloalkyl, alkenyl, alkynyl, cycloalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and
wherein at least one of X₁, X₂, and X₃ is substituted with —COH;

R₁₃, R₁₄, R₁₅, R₁₆, and R₁₇ are each independently selected from the group consisting of hydrogen, alkoxy, haloalkyl, alkenyl, alkynyl, halo, C₁-C₆ alkyl, and C₁-C₃ alkyl-alkoxy;

R₁₈ is selected from the group consisting of hydrogen, haloalkyl, alkenyl, alkynyl, and linear or branched C₁-C₆ alkyl,

wherein the composition is synergistic, in admixture with a carrier.

250. The method of embodiment 249 wherein the composition contains about 0.0001 to 100000 parts per million of a compound of Formula VII.
251. The method of embodiment 249 or 250, wherein R₃₀, R₃₁, R₃₂, and R₃₄ are each hydrogen.
252. The method of any one of embodiments 249-251, wherein R₃₃ is

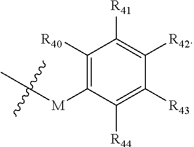

253. The method of any one of embodiments 249-252, wherein R₄₀, R₄₁, R₄₂, R₄₃, and R₄₄ are each hydrogen.
254. The method of any one of embodiments 249-253, wherein M is O.
255. The method of any one of embodiments 249-254, wherein R₂₈ is hydrogen.
256. The method of any one of embodiments 249-255, wherein said compound of Formula VII is

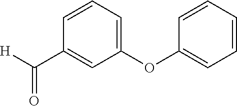 [SMILES: O=Cc1cccc(Oc2ccccc2)c1]

257. The method of any one of embodiments 249-256, wherein said composition comprises

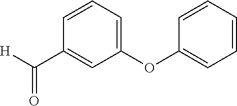 [SMILES: O=Cc1cccc(Oc2ccccc2)c1]

and citronella oil.
258. The method of any one of embodiments 249-257, wherein said composition comprises

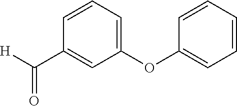 [SMILES: O=Cc1cccc(Oc2ccccc2)c1]

and a compound of Formula VI.
259. The method of any one of embodiments 249-259, wherein said composition comprises

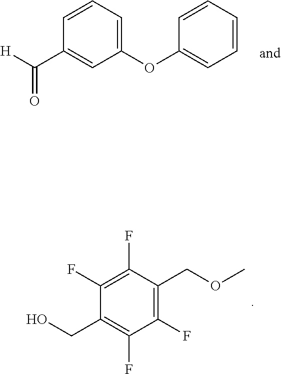

260. The method of any one of embodiments 249-259, wherein said composition comprises

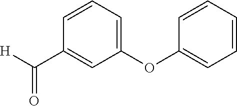 [SMILES: O=Cc1cccc(Oc2ccccc2)c1]

and a compound of Formula II′.
261. The method of any one of embodiments 249-260, wherein said composition comprises

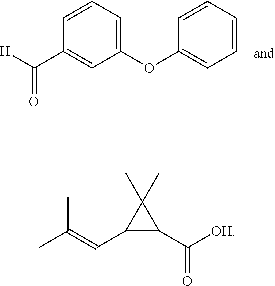

262. The method of any one of embodiments 249-261, wherein said composition comprises

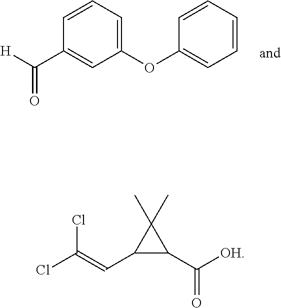

263. The method of any one of embodiments 249-262, wherein the composition is a solution, dust, granular formulation or emulsion.
264. The method of any one of embodiments 249-263, wherein the composition is a liquid solution or emulsion.
265. The method of any one of embodiments 249-264, wherein the composition contains about 0.0001 to 100000 parts per million of a compound of Formula VII.
266. The method of any one of embodiments 249-265, wherein said arthropod is a fly, spider, butterfly, crab, mosquito, centipede, tick, millipede, or scorpion.
267. The method of any one of embodiments 249-266, wherein said arthropod is a mosquito.
268. The method of any one of embodiments 249-267, where the object or locus is an area, an environment, or the skin of an animal.
269. A method of repelling an arthropod from an object or locus, comprising contacting said object or locus with a composition comprising a compound of Formula I′

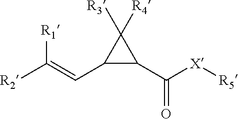

I′ and a compound of Formula II′ or a compound of Formula III′

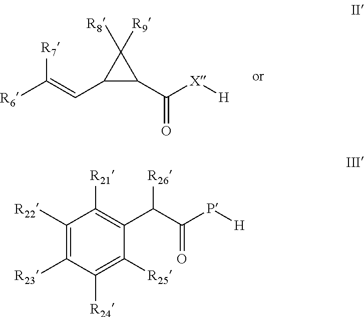

wherein R_{1′} and R_{2′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{3′} and R_{4′} are each independently selected from hydrogen, haloalkyl, alkenyl, alkynyl, and C₁-C₆ alkyl;

R_{5′} is selected from the group consisting of linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X′ is selected from the group consisting of O, NH, and S;

R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl,

wherein the composition is synergistic, in admixture with a carrier.

270. The method of embodiment 269, wherein the composition contains about 0.0001 to 100000 parts per million of a compound of Formula I′.
271. The method of embodiment 269 or 270, wherein R_{3′} and R_{4′} are each methyl.
272. The method of any one of embodiments 269-271, wherein X′ is O.
273. The method of any one of embodiments 269-272, wherein R_{5′} is linear or branched C₁-C₆ alkyl.
274. The method of any one of embodiments 269-273, wherein the composition is a solution, dust, granular formulation, or emulsion.
275. The method of any one of embodiments 269-274, wherein the composition is a liquid solution or emulsion.
276. The method of any one of embodiments 269-275, wherein the composition contains about 0.0001 to 100000 parts per million of the compound of Formula I′.
277. The method of any one of embodiments 269-276, wherein the composition contains a compound of Formula I′ and a compound of Formula II′.
278. The method of any one of embodiments 269-277, wherein X″ is O.
279. The method of any one of embodiments 269-278, wherein R_{6′} and R_{7′} are each independently selected from halo or C₁-C₆ alkyl.
280. The method of any one of embodiments 269-279, wherein R_{8′} and R_{9′} are each methyl.
281. The method of any one of embodiments 269-280, wherein the compound of Formula II″ is

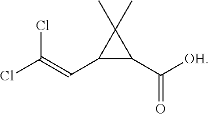

282. The method of any one of embodiments 269-281, wherein the composition comprises a compound of Formula I′ and

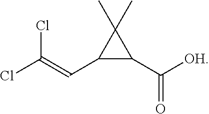

283. The method of any one of embodiments 269-282, wherein the compound of Formula I′ is

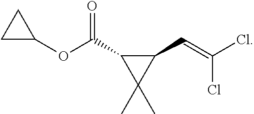

284. The method of any one of embodiments 269-283, wherein said arthropod is a fly, spider, butterfly, crab, mosquito, centipede, tick, millipede, or scorpion.
285. The method of any one of embodiments 269-284, wherein said arthropod is a mosquito.
286. The method of any one of embodiments 269-285, where the object or locus is an area, an environment, or the skin of an animal.
287. A method for controlling one or more arthropods, comprising exposing said one or more arthropods to a synergistic composition comprising a compound of Formula II′ or a compound of Formula III′:

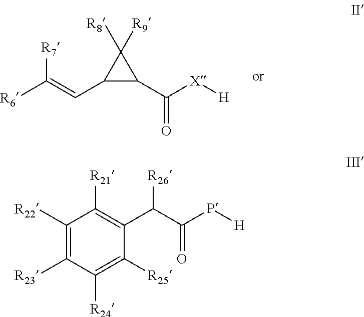

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;

R_{8′} and R_{9′} are each independently selected from hydrogen, haloalkyl, alkenyl, alkynyl, and C₁-C₆ alkyl;

X″ is selected from the group consisting of O, NH, and S;

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;

P′ is selected from the group consisting of O, NH, and S;

R_{26′} is selected from the group consisting of hydrogen, haloalkyl, alkenyl, alkynyl, and linear or branched C₁-C₆ alkyl; and

a pyrethroid,

wherein the synergistic composition produces, when said one or more arthropods are exposed to the synergistic composition, a combined toxicant effect greater than the sum of the separate toxicant effects from the compound of Formula II′ or Formula III′ and the pyrethroid, at comparable concentrations.

288. The method embodiment 287, wherein said toxicant effect is the vapor toxicity of the synergistic composition.
289. The method of embodiment 287 or 288, wherein the synergistic composition contains about 0.0001 to 100000 parts per million of a compound of Formula II′ or Formula III′.
290. The method of any one of embodiments 287-289, wherein said synergistic composition comprises a compound of Formula II′ and transfluthrin.
291. The method of any one of embodiments 287-290, wherein X″ is O.
292. The method of any one of embodiments 287-291, wherein R_{6′} and R_{7′} are selected from halo or C₁-C₆ alkyl.
293. The method of any one of embodiments 287-292, wherein R_{8′} and R_{9′} are each independently C₁-C₃ alkyl.
294. The method of any one of embodiments 287-293, wherein R_{8′} and R_{9′} are each methyl.
295. The method of any one of embodiments 287-294, wherein said compound of Formula II is

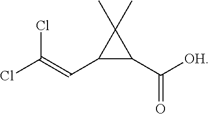

296. The method of any one of embodiments 287-295, wherein said composition comprises

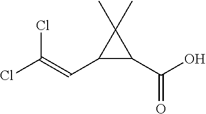

and transfluthrin.
297. The method of any one of embodiments 287-296, wherein the synergistic composition is a solution, dust, granular formulation or emulsion.
298. The method of any one of embodiments 287-297, wherein the synergistic composition is a liquid solution or emulsion.
299. The method of any one of embodiments 287-298, wherein the synergistic composition contains about 0.0001 to 100000 parts per million of a compound of Formula II′.
300. The method of any one of embodiments 287-299, wherein said one or more arthropods are flies, spiders, butterflies, crabs, mosquitos, centipedes, ticks, millipedes, or scorpions.
301. The method of any one of embodiments 287-300, wherein said one or more arthropods are mosquitos.
302. A method of repelling an arthropod from an object or locus, comprising contacting said object or locus with a composition comprising a compound of Formula II, Formula III, Formula IV, Formula V, Formula VI, or Formula VII

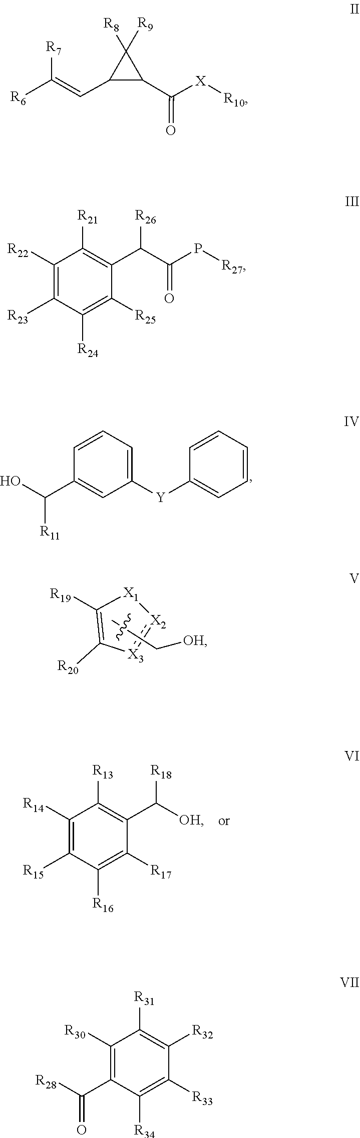

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₂ heterocycloalkyl;

R₈ and R₉ are each independently selected from hydrogen haloalkyl, alkenyl, alkynyl, and C₁-C₆ alkyl;

R₁₀ is selected from the group consisting of hydrogen, haloalkyl, alkenyl, alkynyl, linear or branched C₁-C₁₂ alkyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, haloalkyl, alkenyl, alkynyl, alkoxy, halo, C₁-C₆ alkyl, and C₁-C₃ alkyl-alkoxy;

P is selected from the group consisting of O, NH, and S;

R₂₆ is selected from the group consisting of hydrogen, haloalkyl, haloalkyl, alkenyl, alkynyl, and linear or branched C₁-C₆ alkyl;

R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, haloalkyl, alkenyl, alkynyl, C₁-C₆ alkyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and
wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

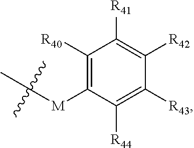

wherein M is selected from the group consisting of O, NH, and S; and

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy,

wherein the composition is synergistic, in admixture with a carrier.

303. The method of embodiment 302, wherein the composition is a solution, dust, granular formulation or emulsion.
304. The method of embodiment 302 or 303, wherein the composition is a liquid solution or emulsion.
305. The method of any one of embodiments 302-304 wherein said arthropod is a fly, spider, butterfly, crab, mosquito, centipede, tick, millipede, or scorpion.
306. The method of any one of embodiments 302-305, wherein said arthropod is a mosquito.
307. The method of any one of embodiments 302-306, wherein the mosquito is Aedes aegypti.
308. The method of any one of embodiments 302-307, where the object or locus is an area, an environment, or the skin of an animal.
309. The method of any one of embodiments 302-308, wherein said compound is of Formula II, wherein X is O and R₁₀ is hydrogen.
310. The method of any one of embodiments 302-209, wherein R₈ and R₉ are each methyl.
311. The method of any one of embodiments 302-310, wherein said compound is selected from the group consisting of

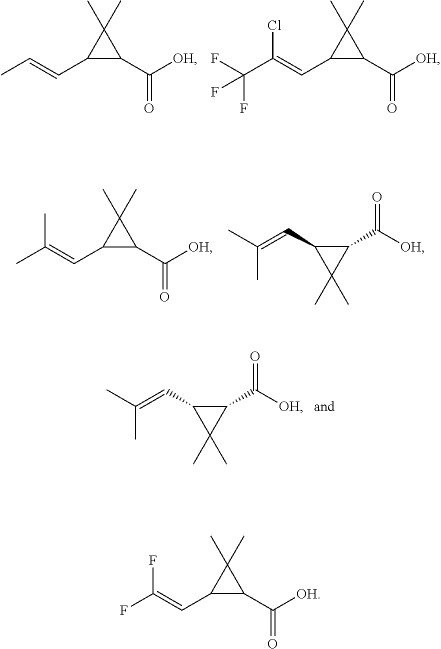

312. The method of any one of embodiments 302-311, wherein said compound is of Formula III.
313. The method of any one of embodiments 302-312, wherein P is O and R₂₇ is H.
314. The method of any one of embodiments 302-313, wherein R₂₃ is halo and R₂₁, R₂₂, R₂₄, and R₂₅ are each hydrogen.
315. The method of any one of embodiments 302-314, wherein R₂₃ is chloro.
316. The method of any one of embodiments 302-315, wherein said compound is

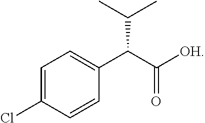

317. The method of any one of embodiments 302-316, wherein said compound is of Formula VI.
318. The method of any one of embodiments 302-317, wherein R₁₃, R₁₄, R₁₆, and R₁₇ are each halo.
319. The method of any one of embodiments 302-318, wherein R₁₃, R₁₄, R₁₆, and R₁₇ are each fluoro.
320. The method of any one of embodiments 302-319, wherein R₁₅ is hydrogen.
321. The method of any one of embodiments 302-320, wherein R₁₈ is hydrogen.
322. The method of any one of embodiments 302-321, wherein the compound of Formula VI is

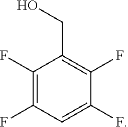 [SMILES: OCc1c(F)c(F)cc(F)c1F]

323. The method of any one of embodiments 302-322, wherein the compound is of Formula IV.
324. The method of any one of embodiments 302-323, wherein Y is O.
325. The method of any one of embodiments 302-325, wherein R₁₁ is CN.
326. The method of any one of embodiments 302-325, wherein the compound of Formula IV is

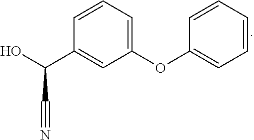 [SMILES: N#C[C@@H](O)c1cccc(Oc2ccccc2)c1]

327. The method of any one of embodiments 302-327, wherein the compound is of Formula VII.
328. The method of any one of embodiments 302-327, wherein R₃₀, R₃₁, R₃₂, and R₃₄ are each hydrogen.
329. The method of any one of embodiments 302-328, wherein R₃₃ is

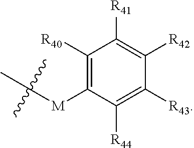

330. The method of any one of embodiments 302-329, wherein R₄₀, R₄₁, R₄₂, R₄₃, and R₄₄ are each hydrogen.
331. The method of any one of embodiments 302-330, wherein M is O.
332. The method of any one of embodiments 302-331, wherein R₂₈ is H.
333. The method of any one of embodiments 302-332, wherein the compound of Formula VII is

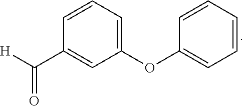 [SMILES: O=Cc1cccc(Oc2ccccc2)c1]

334. The method of any one of embodiments 302-333, wherein the compound is of Formula II, wherein X is O and R₁₀ is selected from C₃-C₆ cycloalkyl and linear or branched C₁-C₆ alkyl.
335. A composition comprising an essential oil; and a second compound selected from the group consisting of a pyrethroid, a compound of Formula II, Formula III, Formula IV, Formula V, Formula VI, and Formula VII

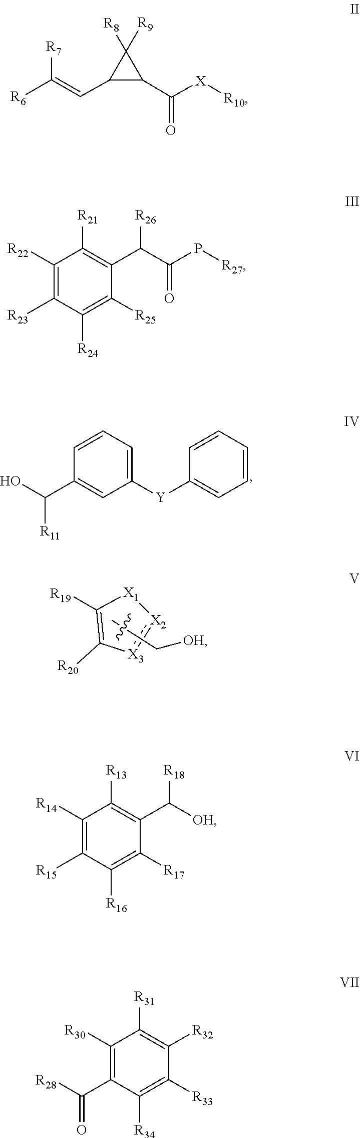

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₈ and R₉ are each independently selected from hydrogen haloalkyl, alkenyl, alkynyl, and C₁-C₆ alkyl;

R₁₀ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, alkoxy, halo, haloalkyl, alkenyl, alkynyl, C₁-C₆ alkyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl;

R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, haloalkyl, alkenyl, alkynyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and
wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

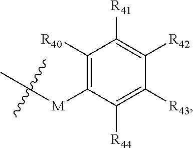

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy,

wherein the composition is synergistic, in admixture with a carrier.

336. The composition of embodiment 335, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.
337. The composition of embodiment 335 or 336, wherein said essential oil is citronella oil.
338. The composition of any one of embodiments 335-337, wherein said second compound is a pyrethroid.
339. The composition of any one of embodiments 335-338, wherein said pyrethroid is selected from the group consisting of permethrin, tetramethrin, metofluthrin, bifenthrin, kadethrin, allethrin, cyfluthrin, deltamethrin, empenthrin, cypermethrin, cyphenothrin, esfenvalerate, fenvalerate, flumethrin, tefluthrin, phenothrin, and transfluthrin.
340. The composition of any one of embodiments 335-339, wherein said pyrethroid is metofluthrin.
341. The composition of any one of embodiments 335-340, wherein said composition comprises a pyrethroid, wherein said pyrethroid is metofluthrin and said essential oil is selected from the group consisting of citronella oil, Canadian balsam, galbanum oil, ginger root oil, dill seed oil, and cypress oil.
342. The composition of embodiment 341, wherein said composition comprises metofluthrin having a concentration of about 0.1 μg/cm², and said essential oil having a concentration selected from the group consisting of 100 μg/cm², 30 μg/cm², and 20 μg/cm².
343. A method of repelling an arthropod from an object or locus, comprising contacting said object or locus with a composition comprising an essential oil, and a second compound selected from the group consisting of a pyrethroid, a compound of Formula II, Formula III, Formula IV, Formula V, Formula VI, and Formula VII

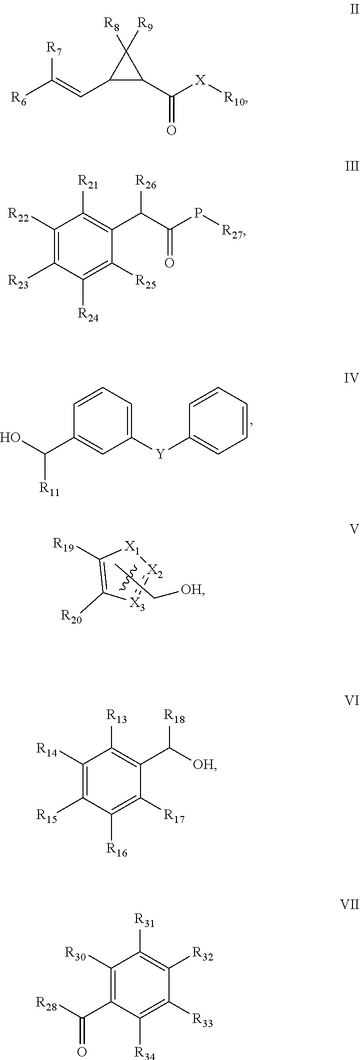

wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or

wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;

R₈ and R₉ are each independently selected from hydrogen haloalkyl, alkenyl, alkynyl, and C₁-C₆ alkyl;

R₁₀ is selected from the group consisting of hydrogen, linear or branched C₁-C₁₂ alkyl, C₃-C₁₂ cycloalkyl, haloalkyl, alkenyl, alkynyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

X is selected from the group consisting of O, NH, and S;

R₂₁, R₂₂, R₂₃, R₂₄, and R₂₅ are each independently selected from the group consisting of hydrogen, alkoxy, halo, C₁-C₆ alkyl, haloalkyl, alkenyl, alkynyl, and C₁-C₃ alkyl-alkoxy;

P is selected from the group consisting of O, NH, and S;

R₂₆ is selected from the group consisting of hydrogen, haloalkyl, alkenyl, alkynyl, and linear or branched C₁-C₆ alkyl;

R₂₇ is selected from the group consisting of hydrogen, linear or branched C₁-C₁₂ alkyl, C₃-C₁₂ cycloalkyl, haloalkyl, alkenyl, alkynyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;

R₁₁ is selected from the group consisting of hydrogen, C₁-C₆ alkyl, alkenyl, alkynyl, CN, halo, and haloalkyl;

R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or

where the substituents on R₁₉ and R₂₀, taken together with the carbon to which each is attached, form a C₅-C₇ cycloalkyl;

X₁, X₂, and X₃ are each independently selected from the group consisting of CH, CH₂, O, S, N, C═O, and NH; and
wherein at least one of X₁, X₂, and X₃ is substituted with —COH;

R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;

R₁₈ is selected from the group consisting of hydrogen, haloalkyl, alkenyl, alkynyl, and linear or branched C₁-C₆ alkyl;

R₂₈ is selected from the group consisting of hydrogen, haloalkyl, alkenyl, alkynyl, alkoxy, and C₁-C₆ linear or branched alkyl;

R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and

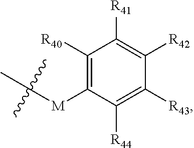

wherein M is selected from the group consisting of O, NH, and S;

R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy,

wherein the composition is synergistic, in admixture with a carrier.

344. The method of embodiment 343, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.
345. The method of embodiment 343 or 344, wherein said essential oil is citronella oil.
346. The method of any one of embodiments 343-345, wherein said second compound is a pyrethroid.
347. The method of any one of embodiments 343-346, wherein said pyrethroid is selected from the group consisting of permethrin, empenthrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.
348. The method of embodiments 343-347, wherein said pyrethroid is metofluthrin.
349. The method of any one of embodiments 343-348, wherein said composition comprises a pyrethroid, wherein said pyrethroid is metofluthrin, and said essential oil is selected from the group consisting of citronella oil, Canadian balsam, galbanum oil, ginger root oil, dill seed oil, and cypress oil.
350. The method of any one of embodiments 343-349, wherein said composition comprises metofluthrin having a concentration of about 0.1 μg/cm², and said essential oil having a concentration selected from the group consisting of 100 μg/cm², 30 μg/cm², and 20 μg/cm².
351. The method of any one of embodiments 343-350, or the composition of any one of embodiments 335-341, wherein said essential oil is citronella oil and said second compound is empenthrin.
352. The method of embodiment 351, wherein said composition comprises empenthrin having a concentration of about 0.1 μg/cm², and said essential oil having a concentration of about 100 μg/cm².
353. A method of repelling an arthropod from an object or locus, comprising contacting said object or locus with any single compound described herein, or a composition comprising any single compound and a carrier.
354. A method for controlling one or more arthropods, comprising exposing said one or more arthropods to any single compound described herein, or a composition comprising any single compound and a carrier.
355. A method of repelling an arthropod from an object or locus, comprising contacting said object or locus with a compound in Table 8, or a composition comprising a compound in Table 8 and a carrier.
356. A method for controlling one or more arthropods, comprising exposing said one or more arthropods to a compound in Table 8, or a composition comprising a compound in Table 8 and a carrier.
357. The method of any one of embodiments 211-356, wherein said arthropod is repelled from an adjacent area to said object or locus, wherein said arthropod is not physically contacted with said object or locus.
358. A method of providing a composition of any one of embodiments 1-357 in an arthropod repellent medium from which a vaporized composition of any one of embodiments 1-357 can be dispersed.
359. The method of any one of embodiments 1-358, wherein the composition is formulated for use in a vaporizer, evaporator, fan, heat, candle, or wicked apparatus.
360. The method of any one of embodiments 1-359, wherein the synergistic composition is a vapor.
361. The method or composition of any one of embodiments 1-360, wherein said arthropod is an insect.
362. The method of any one of embodiments 1-361, wherein said insect is repelled from an adjacent area to said object or locus, wherein said insect is not physically contacted with said object or locus.
363. A method of providing a composition of any one of embodiments 1-362 in an insect repellent medium from which a vaporized composition of any one of embodiments 1-362 can be dispersed.

The following examples are offered by way of illustration and not by way of limitation.

### EXAMPLES

### Example 1: Assay for Determining Repellency and Vapor Toxicity with DEET as Positive Control

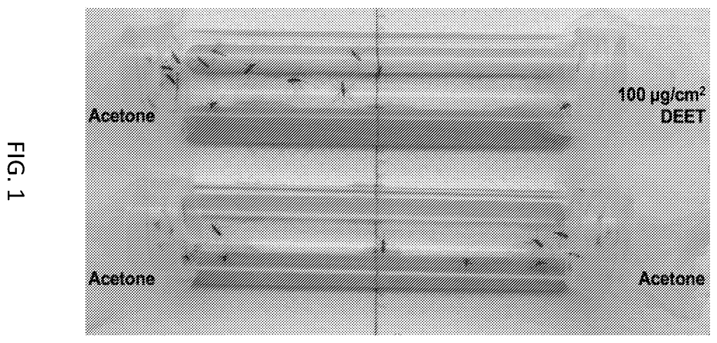

The following assay was conducted according to the procedures of Jiang et al., 2019 (Jiang S., Yang L, and Bloomquist J (2019) High-throughput screening method for evaluating spatial repellency and vapor toxicity to mosquitoes. Med. Vet. Entomol.). For assessing behavioral repellency (FIG. 1), sixteen female Aedes aegypti mosquitoes were transferred to a glass tube (12.5 cm long, 2.5 cm outer diameter) with the ends covered with netting. Metofluthrin, bifenthrin, permethrin, pyrethrum, transfluthrin, deltamethrin, esfenvalerate, metofluthrin acid, bifenthrin acid, phenothrin acid, transchryanthemic acid, transfluthrin acid, deltamethrin acid, and fenvalerate acid were each dissolved in 1 mL of acetone and serial dilutions ranging from 30-0.01 μg/ml were made. Circular filter papers (5 cm²) were saturated with 50 μL of each concentration and allowed 10 min for solvent evaporation. The filter papers were then placed in clear conical polypropylene caps and fixed to the end of the glass tubes. The netting prevented mosquito contact with the filter paper. Tubes were then aligned with a mark denoting the midline and the proportion of mosquitoes on the chemically treated side was scored at 15, 30, and 60 min. At the end of the test, the tube was placed in a cage and the ability of the mosquitoes to fly out of the tube was assessed for 30 min. For controls, after a 60 min observation period, all the mosquitoes left the tube within 10 min. This post-test evaluated whether any non-responding mosquitoes (not repelled) was due to intoxication or disorientation from the test chemical. For vapor toxicity, the assay was identical, except that mosquitoes were kept in the tube for 24 hr without the post-test at 1 hr, and the mortality was recorded at 24 hr.

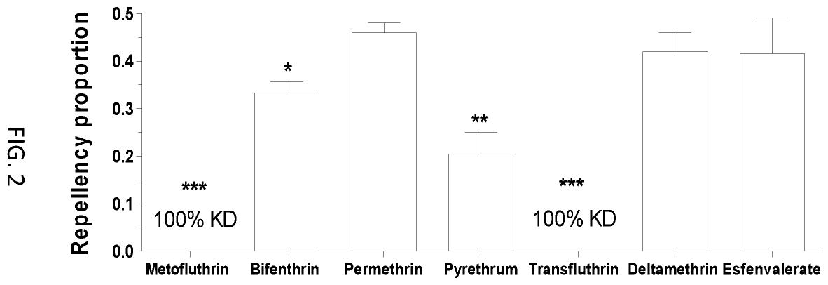

As shown in FIG. 2, the spatial repellency of each of the compounds investigated exhibited low activity. Although metofluthrin and transfluthrin displayed significant mortality, they did show repellency at lower level exposures. Furthermore, bifenthrin and pyrethrum exhibited some repellency at high exposure, while permethrin and deltamethrin were inactive as vapor phase repellents.

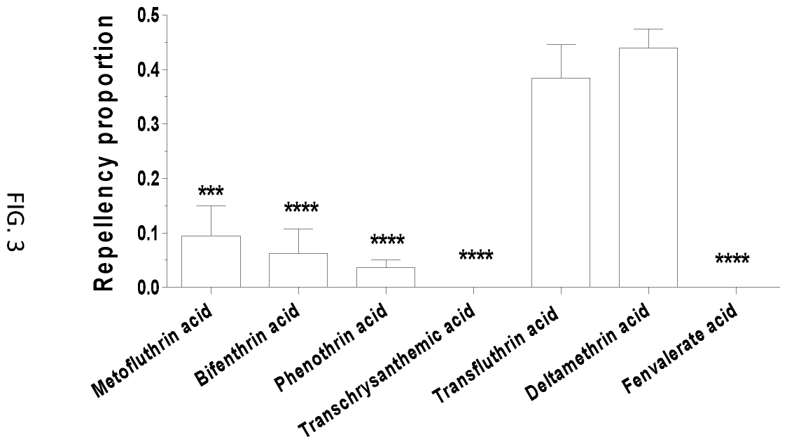

It was determined that the acid form of the compounds investigated was more active as spatial repellents than their respective parent compound, as shown in FIG. 3 and FIG. 4, as well as Tables 1 and 2. Exceptions were transfluthrin acid (TFA) and metofluthrin acid.

The repellency of the pyrethroid acids additionally exhibited much less cross resistance in the PR Puerto Rico (pyrethroid-resistant) strain of Ae. aegypti, unlike their parent compounds. Table 1 shows one hr repellency EC₅₀ values in g/cm² for some pyrethroid acids. EC₅₀ values were calculated from non-linear regression to a four parameter logistic equation (Prism, GraphPad Software, San Diego, Calif.) of the curves shown in FIG. 4. RR=resistance ratio. NA=data not yet available. Worthwhile noting are the low levels of resistance to repellency by pyrethroid acids in the insecticide-resistant PR strain of Ae. aegypti.

TABLE 1

| Chemical | MFA | BFA | PhenA | TCA | FTFA | FVA |
|---|---|---|---|---|---|---|
| 1 h EC₅₀-OR | 14 | 14 | 19 | 19 | 27 | 19 |
| 1 h EC₅₀-PR | 18 | 11 | 15 | 36 | 35 | 32 |
| RR (PR EC₅₀/OR EC₅₀) | 1.3 | 0.8 | 0.8 | 1.9 | 1.3 | 1.7 |

Table 2 displays the repellency EC₅₀ in OR and PR (g/cm²) for several intact compounds, which exhibited significant vapor repellency.

TABLE 2

| Chemical | Metofluthrin | Pyrethrum | Transfluthrin |
|---|---|---|---|
| 1 h EC₅₀-OR | 0.26 | 31 | 0.49 |
| 1 h EC₅₀-PR | 15.5 | 311 | 2.28 |
| RR (PR EC₅₀/OR EC₅₀) | 60 | 11 | 4.7 |

As shown in Tables 1 and 2, the acids exhibited substantially more repellency compared to their corresponding intact parent compounds, most of which showed little or no activity except for transfluthrin and metofluthrin, which were designed to work in the vapor phase. Moreover, the spatial activity of pyrethrum was about equal to that of one of its components, TCA, against OR (Orlando) mosquitoes. Additionally, the cross resistance (RR) to the acids was much less.

### Example 2: Synergy Experiments with TFA

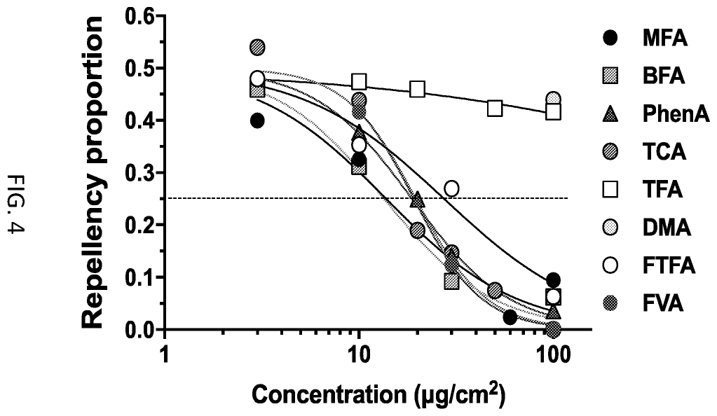

Repellents/contact irritants were screened for synergism in mixtures containing an inactive level of TFA (50 μg/cm²)+EC₂₀ treatment of test compound with the parentheses ( ) in FIG. 5 denoting the actual concentration in μg/cm² and assessed at 1 hr. Permethrin and VUAA-1 were tested at 30 and 100 μg/cm² respectively, to illustrate the synergistic effect, although they were inactive at 1 mg/cm². For these experiments, TFA was selected because it exhibited no repellent activity by itself, even at high concentrations (FIG. 4), so it provided a good baseline material for comparing the effects of mixtures. Compounds were applied alone, or as an acetone-based mixture pipetted onto the filter paper. VUAA-1 is an experimental repellent with a unique action on olfactory receptors, in vitro (Jones P, Pask G, Rinker D, and Zweibel L (2011) Functional agonism of insect odorant receptor ion channels. Proc. Natl. Acad. Sci. USA 108:8821-8825.), but is only vapor-detectable by mosquito antennae when heated to high temperatures (L. Yang, Y. Liu, G. M. Richoux, U. R. Bernier, K. R. Linthicum, J. R. Bloomquist (2019) Induction coil heating improves the efficiency of insect olfactory studies. Frontiers in Ecology and Evolution 7, 247; doi: 10.3389/fevo.2019.00247.). Data were also collected for the contact irritant, permethrin, which when combined with TFA, exhibited some spatial repellency. Without wishing to be bound by theory, it is understood that one possible action of TFA is to help volatilize compounds off of the filter. It is worthwhile noting that there was no statistically significant effect on either DEET or IR3535 repellency when applied as a mixture.

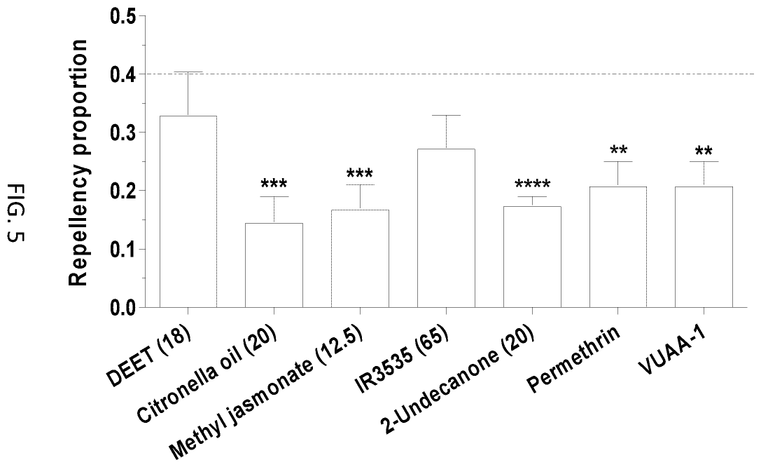

As shown in FIG. 6, an experiment with TFA and TCA promoted a shift in the concentration-response curves for 2-undecanone. TFA at 50 μg/cm² had no effect and TCA at 10 μg/cm² had 25% repellency. Thus, the synergism observed for TFA can be extended to other pyrethroid acids, as well.

Provided in Table 3 are the quantified repellency of standards that responded positively to the presence of 50 μg/cm² of TFA after 1 hr. Citronella, methyl jasmonate, and 2-undecanone are natural products with insect repellent properties. EC₅₀ values in μg/cm² were determined as described in Table 1 and synergist ratios (SR) were calculated as EC₅₀ compound alone/EC₅₀ for compound+TFA. All compounds were synergized by TFA, with very large effects on VUAA-1 and permethrin.

TABLE 3

| | | methyl | | | |
|---|---|---|---|---|---|
| | citronella | jasmonate | ^{a}2-undecanone | VUAA1 | permethrin |
| EC₅₀ | 33 | 39 | 59 | >2000 | >1000 |
| EC₅₀ (+TFA) | 10 | 6 | 5.1 | 30 | 9.5 |
| SR | 3.3 | 6.5 | 11.6 | >67 | >105 |

^{a}2-undecanone + TCA gave an EC₅₀ of 7 μg/cm² and a synergist ratio of 8.4.

### Example 3: TFA and TCA Synergism in a Human Arm Bite Protection Assay

A human bite protection assay was performed according to see if the repellency observed in glass tubes extended to bite protection. The assay was run in accordance with that of Tsikolia et al. (Tsikolia M, Bernier UR, Coy M, Chalaire K, Becnel J, Agramonte N, Tabanca N, Wedge D, Clark G, Linthicum K, Swale D, and J. R. Bloomquist J (2013) Insecticidal and fungicidal properties of novel trifluoromethylphenyl amides. Pestic. Biochem. Physiol. 107, 138-147). The results are provided in Table 4. Treated cloth patches were worn by human volunteers and the number of blood fed mosquitoes was determined after 1 min spent in a cage with 500 (±10%) female Ae. aegypti. Minimum Effective Dosage (MED in mg/cm²) is the minimum amount required to prevent 99% of bites in three human volunteers (UF IRB #201602334). Synergist ratio (SR)=MED chemical alone/MED chemical+TFA (0.047 mg/cm²) or TCA (0.094 mg/cm²). Thus, the synergism observed in the tube assays translated well to bite protection of human arms and was observed for both contact and spatial repellents.

TABLE 4

| | MED | MED | MED | | | |
|---|---|---|---|---|---|---|
| Compound | values | values | values | | | |
| Treatment | Male 4 | Female 9 | Female 10 | Mean | SEM | SR |
| DEET | 0.011 | 0.011 | 0.011 | 0.011 | 0 | — |
| methyl jasmonate | >1.5 | 0.75 | 0.75 | >1 | — | — |
| citronella | 0.047 | 0.047 | 0.047 | 0.047 | 0 | — |
| IR3535 | 0.094 | 0.047 | 0.047 | 0.063 | 0.016 | — |
| 2-undecanone | 0.094 | 0.187 | 0.375 | 0.219 | 0.083 | — |
| permethrin | 0.187 | 0.187 | 0.187 | 0.187 | 0.00 | |
| 3-phenoxybenzylaldehyde | >0.75 | >0.75 | 0.750 | >0.75 | | |
| TFA | 0.187 | 0.187 | 0.094 | 0.156 | 0.031 | — |
| TCA | 0.375 | 0.187 | 0.375 | 0.312 | 0.063 | — |
| DEET + TFA | 0.005 | 0.005 | 0.005 | 0.005 | 0 | 2.2 |
| methyl jasmonate + TFA | 0.047 | 0.047 | 0.023 | 0.039 | 0.008 | >25 |
| citronella + TFA | 0.011 | 0.005 | 0.0013 | 0.006 | 0.003 | 7.8 |
| IR3535 + TFA | 0.047 | 0.011 | 0.047 | 0.035 | 0.012 | 1.8 |
| 2-undecanone + TFA | 0.047 | 0.023 | 0.047 | 0.039 | 0.008 | 5.6 |
| 2-undecanone + TCA | 0.023 | 0.011 | 0.023 | 0.019 | 0.004 | 11.5 |
| permethrin + TFA | 0.011 | 0.005 | 0.002 | 0.006 | 0.003 | 31.2 |
| 3-phenoxybenzaldehyde + | 0.023 | 0.023 | 0.023 | 0.023 | 0.00 | 32.6 |
| TFA | | | | | | |

### Example 4: Examination of How Compounds Deployed on Filter Affect Synergistic Activity

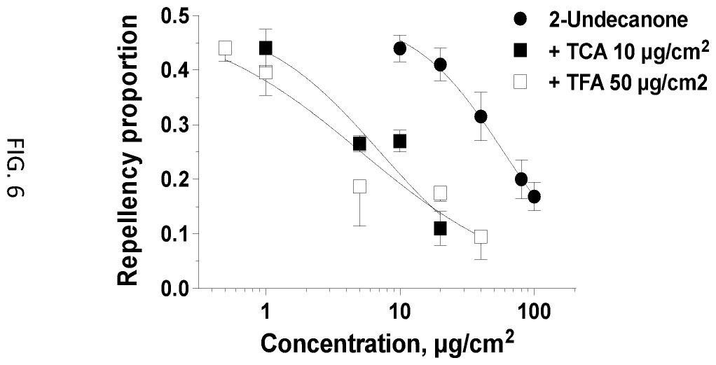

It is generally known in the art that DEET can “trap” some odorants on surfaces (Syed Z and Leal W (2008) Mosquitoes smell and avoid the insect repellent DEET. Proc. Natl. Acad. Sci. USA 105: 13598-13603.). As such, experiments were performed to see if treating each half of a filter paper produced the same results as the mixture experiment in Example 3 (FIGS. 7A and 7B). Repellency was monitored when DEET (18 ag/cm²) was applied side-by-side (S) or mixed (M) with TFA (50 μg/cm²). A synergistic effect was observed when DEET and TFA were applied in their fixed concentrations side-by-side. As shown in the concentration-response plot (FIG. 7 B), for the mixture experiment, as the DEET concentration declined, the curve deviated progressively from that of DEET alone, and showed more repellency at 10 and 20 μg/cm² as the DEET “trapping effect” presumably declined. In this experiment, the EC₅₀ for DEET was 39 μg/cm², +TFA(M) was 26 μg/cm², and +TFA(S) was 8.1 μg/cm². Thus, the addition of TFA in the mixture decreased the EC₅₀ value 1.5-fold, while the side-by-side treatment decreased it nearly 5-fold, indicating significantly better DEET repellent performance in the presence of TFA. Worthwhile noting is that when side-by-side treatment was performed with TFA and VUAA-1, the synergism disappeared. In this manner, the deployment of mixtures depends on the compounds of interest.

### Example 5: Analysis of TFA's Modes of Repellent Action

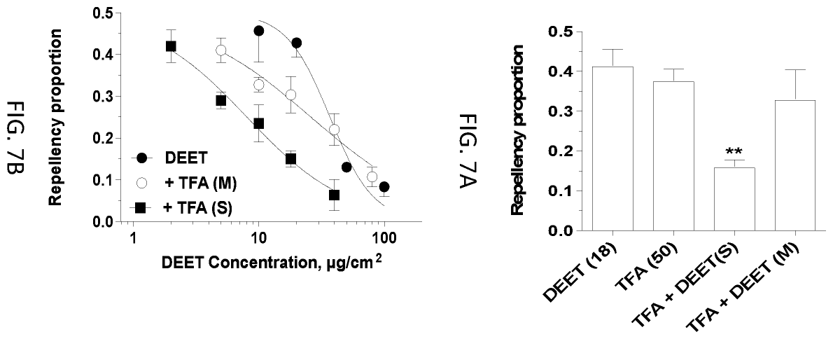

The repellency of transfluthrin (TF) alone and when mixed with TFA was examined in FIG. 8. The EC₅₀ for transfluthrin alone was 0.5 μg/cm² and 0.11 when mixed with TFA. Thus, the synergism ratio was about 5.

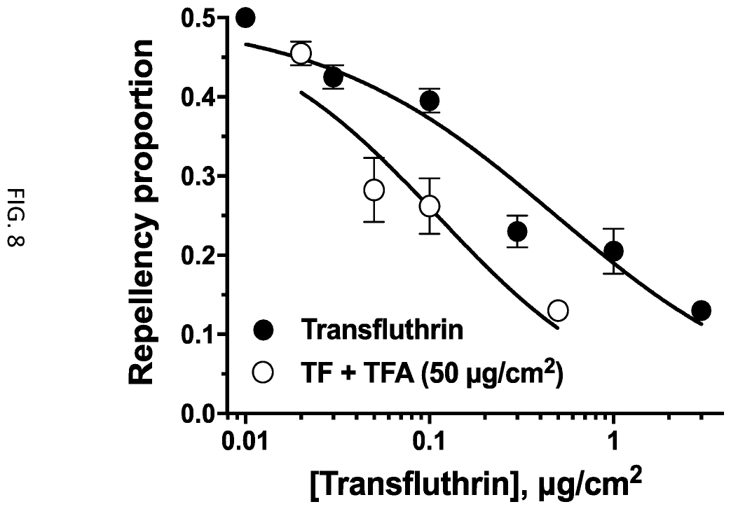

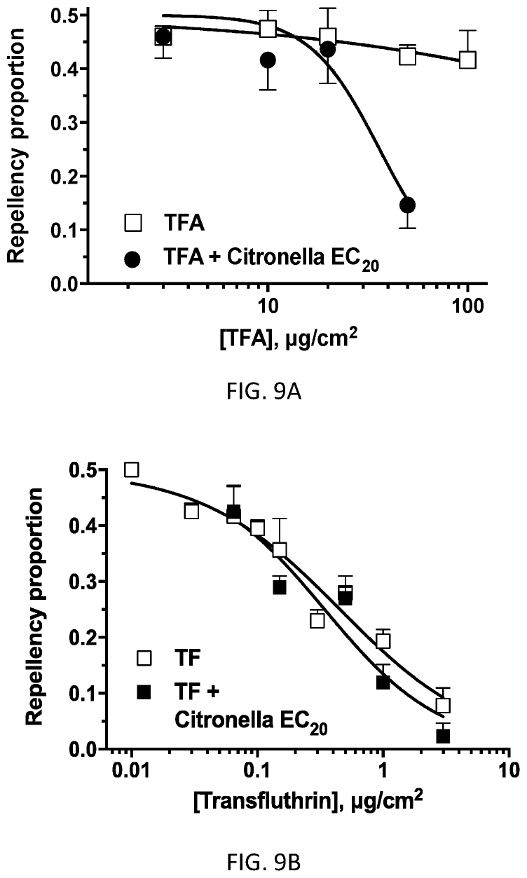

The synergistic interactions of TFA and citronella and transfluthrin (TF) and citronella were assessed in FIG. 9A and FIG. 9B, respectively. Synergistic interactions were discovered between TFA and citronella, but not between transfluthrin and citronella. This finding supports different modes of action of TFA and TF.

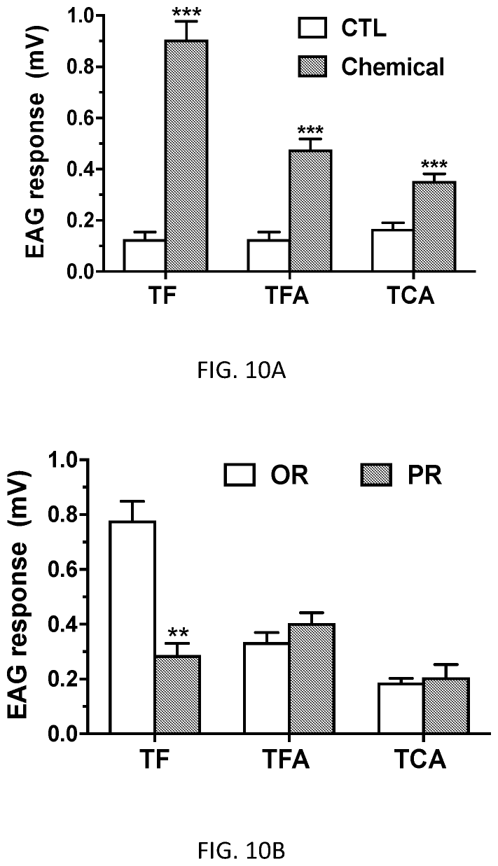

Electroantennography (EAG) responses of OR (susceptible) and PR (resistant) strains of Ae. Aegypti were then obtained for TF, TFA, and TCA (FIGS. 10A and 10B). In these experiments, an air puff stimulus with odorant was blown over an antenna suspended between 2 electrodes and represented the summed electrical response of all the chemo-receptive hairs. All compounds gave responses, with TF the largest (FIG. 10A). EAG responses to TF were reduced in the resistant PR strain (**P<0.01), but not to TFA or TCA, demonstrating the lack of cross resistance to the pyrethroid acids, but not TF (FIG. 10B).

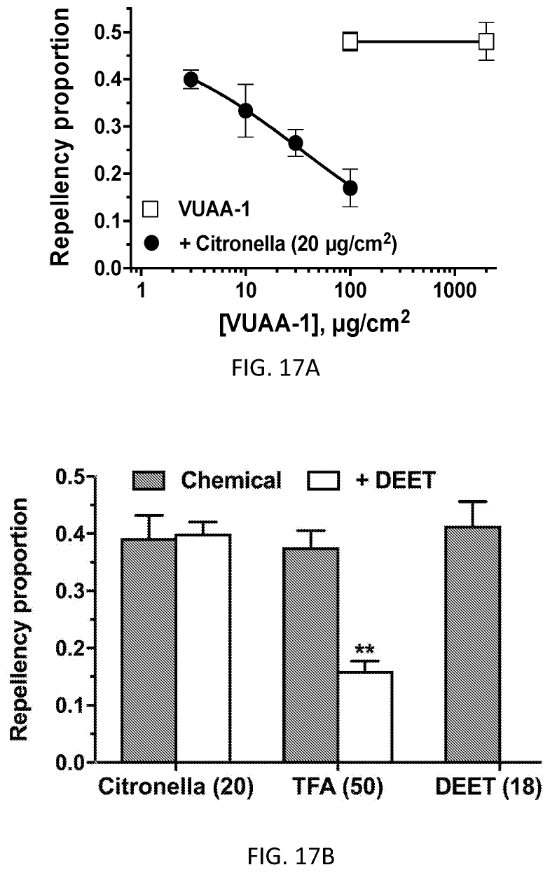

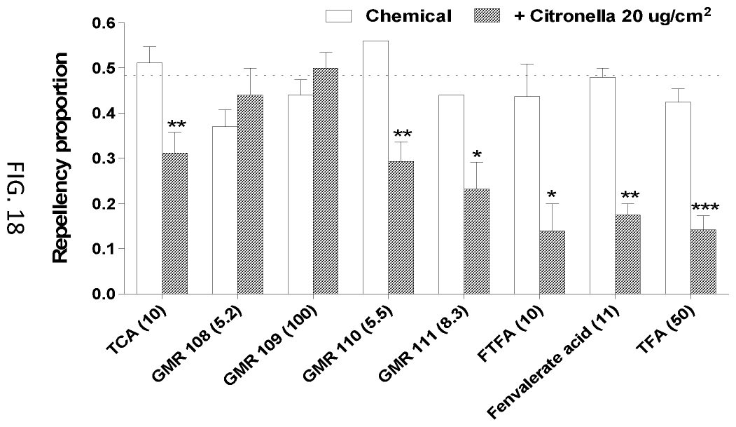

The manner in which Citronella synergizes VUAA-1, but not that of DEET was compared to TFA (FIGS. 17A and 17B). Citronella at its EC₂₀ greatly enhanced the spatial repellency of VUAA-1 (EC₅₀=34 μg/cm²), similar to TFA (FIG. 7 and Table 3). In contrast, it did not synergize the repellency of DEET, unlike TFA in matched experiments (concentration in μg/cm²).

### Example 6: Pyrethroid Alcohols Demonstrate Repellent Activity Against Female Ae. Aegypti

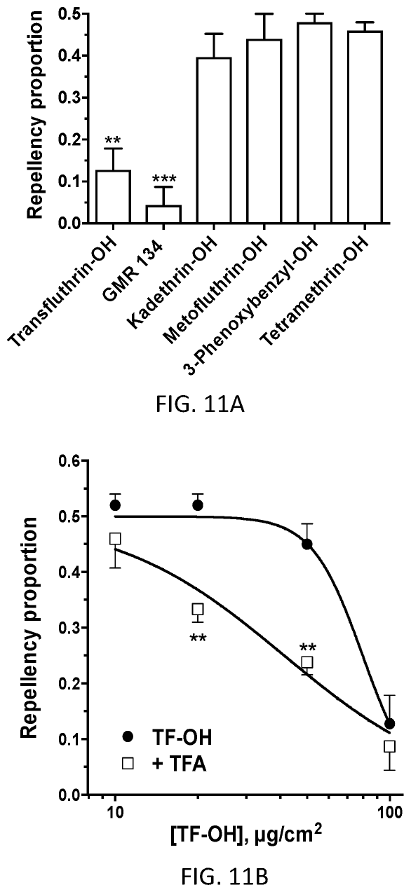

Pyrethroid alcohols were used to obtain repellency screening along and in combination with TFA (FIGS. 11A and 11B). The pyrethroid alcohols were screened at 100 μg/cm². Both transfluthrin alcohol and GMR 134 showed significant repellency compared to the controls (**P<0.01; ***P<0.001). As shown in FIG. 11B, transfluthrin alcohol (TF-OH) was synergized by 50 μg/cm² TFA. The EC₅₀ for TF-OH was 80 μg/cm² and in the presence of TFA was 41 μg/cm², giving a synergist ratio of about 2.

### Example 7: Pyrethroid Acids, Alcohols, and Aldehydes Show Synergistic Repellency

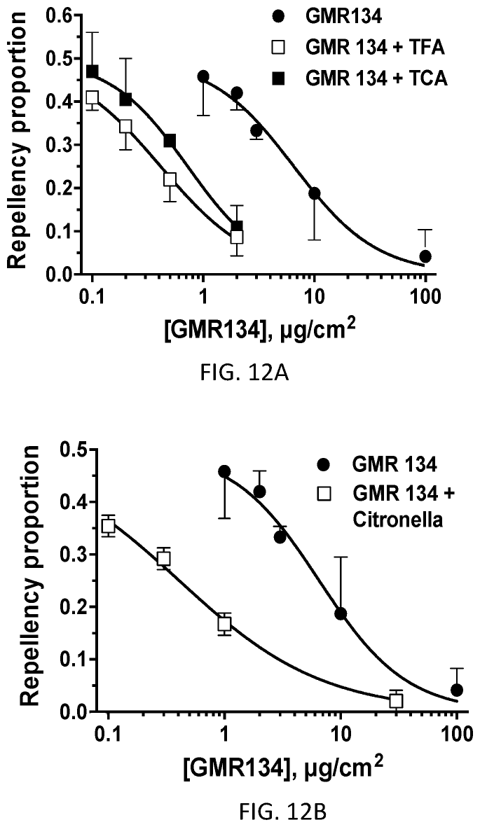

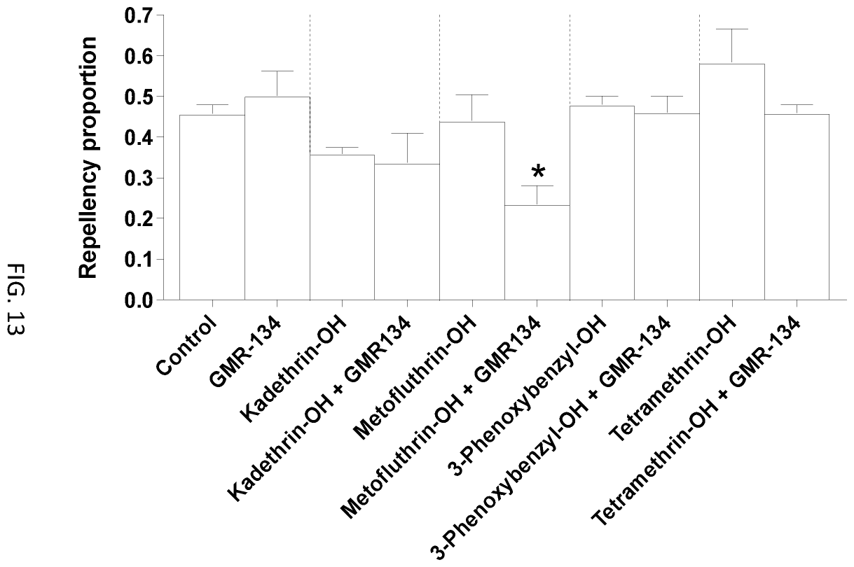

Chemical analysis found that instability resulted in the rearrangement of GMR134 to its corresponding aldehyde. The aldehyde exhibited good repellency, as shown in FIGS. 12A and 12B, which gave excellent performance in combination with pyrethroid acids and citronella. The data in FIGS. 12A and 12B gave synergism ratios for TFA, TCA, and citronella of 16, 9.3, and 16, respectively.

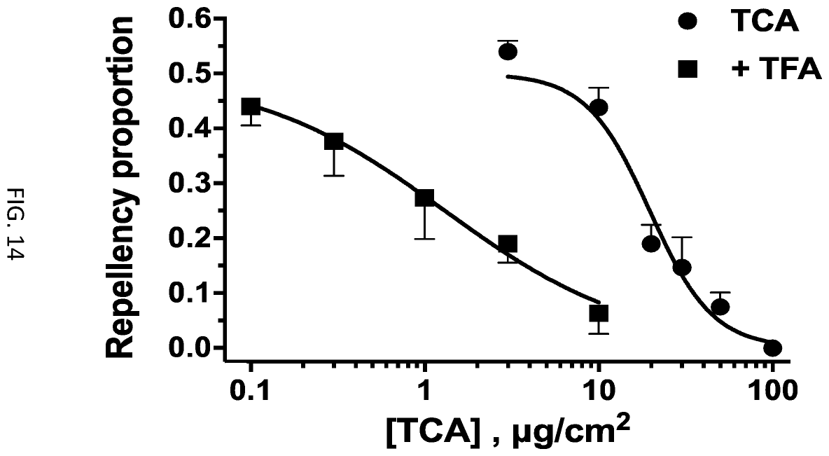

Because GMR 134 had good repellency and synergism with pyrethroid acids and citronella, it was tested in mixtures with pyrethroid alcohols (FIG. 13). It was determined that only metofluthrin alcohol (metofluthrin-OH) significantly synergized GMR 134 (*P<0.05).

It was next documented that different pyrethroid acids can synergize one another (FIG. 14).

### Example 8: TFA Esters Show Repellency Against Ae. aegypti Females

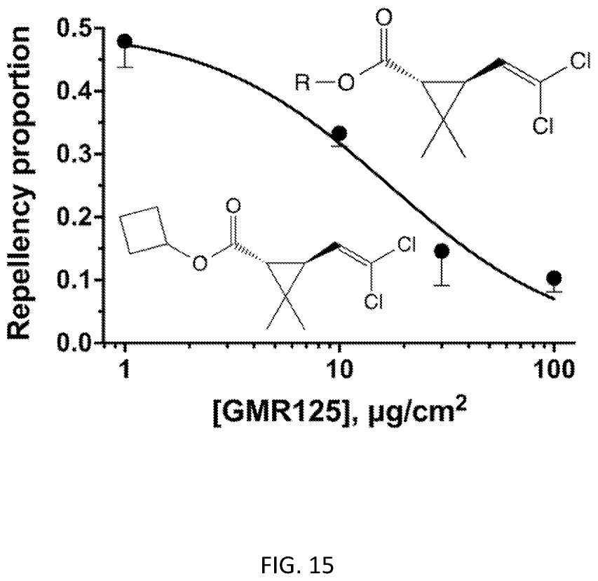

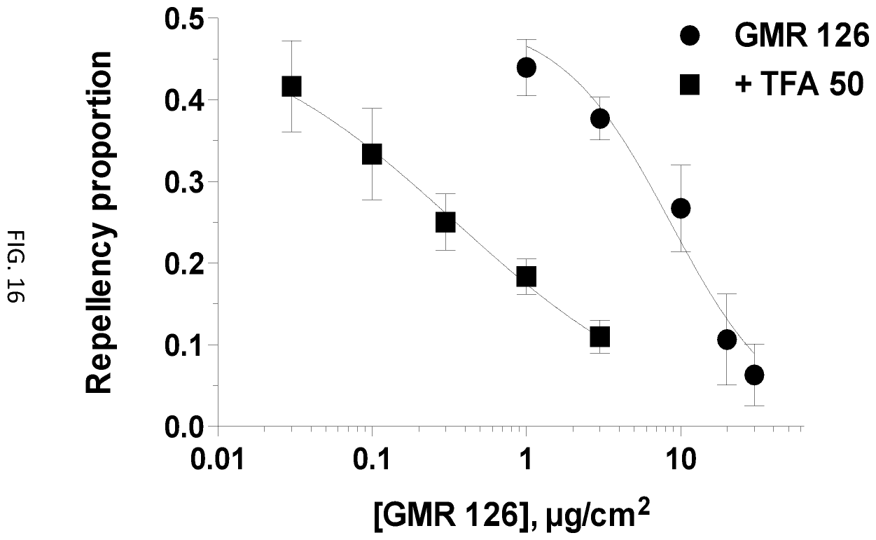

Table 5 shows EC₅₀ values for repellency of experimental TFA esters in 1 hr exposures that displayed little knockdown at a screening concentration of 100 μg/cm². FIG. 15 shows a representative concentration-response curve for GMR125, along with a generic structure for TFA esters and the structure of GMR 125. EC₅₀ values are in μg/cm². GMR 088, 115, 116 and 082, showed considerable knockdown at 1 hr when tested at 100 μg/cm².

TABLE 5

| Compound | R | EC₅₀ (95% CL) |
|---|---|---|
| GMR 120 | n-pentyl | 10 (7-15) |
| GMR 121 | n-butyl | 24 (12-45) |
| GMR 122 | t-butyl | 25 (12-57) |
| GMR 124 | cyclopentyl | 11 (8-15) |
| GMR 125 | cyclobutyl | 17 (11-27) |
| GMR126 | cyclopropyl | 8.5 (5-13) |

### Example 9: Synergism Screening of Pyrethroid Acids with Citronella

Screening results of TFA esters for synergism with citronella are provided in FIG. 18. The pyrethroid acids synergized citronella, but the tested esters did not, and this lack of effect was consistent with that observed for the pyrethroid, transfluthrin (FIGS. 9A and 9B).

### Example 10: Synergism Screening of Pyrethroid Esters with Citronella

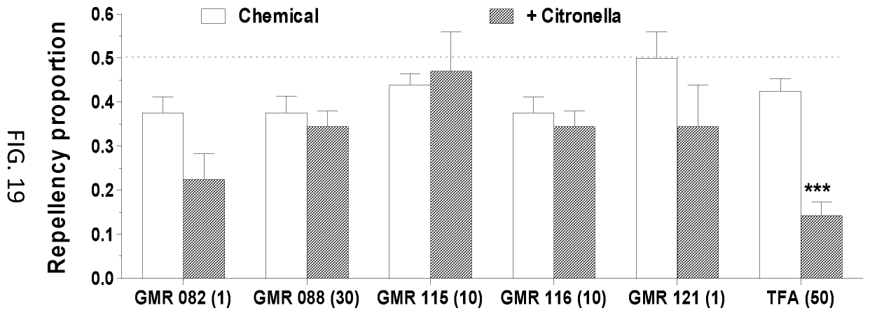

The results of screening TFA esters for synergism with citronella are provided in FIG. 19. The pyrethroid acids synergized citronella, but the tested esters did not, and this lack of effect was consistent with that observed for the pyrethroid, transfluthrin (FIGS. 9A and 9B).

### Example 11: Pyrethroid Acids and Citronella Synergize Vapor Toxicity of Pyrethroids

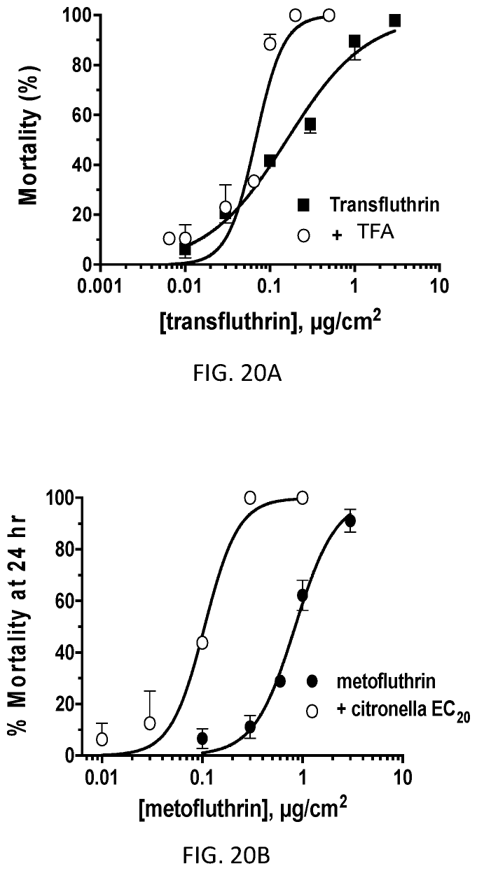

As shown in FIGS. 20A and 20B, TFA (50 μg/cm²) synergized the 24 h toxicity of transfluthrin, and citronella synergized the toxicity of metofluthrin. Transfluthrin LC₅₀=0.16 μg/cm² and +TFA=0.07 μg/cm², yielding a synergism ratio=2.3. Similarly, metofluthrin LC₅₀=0.84 μg/cm² and +citronella=0.11 μg/cm², giving a synergism ratio=7.6. Additional investigations describing the synergy of metrofluthrin with citronella oil are described in Example 17.

### Example 12: Synergistic Compositions of GMR 134 with TFA

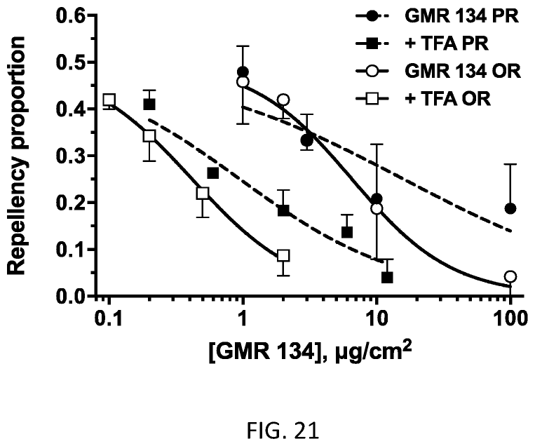

Concentration-response curves for repellent mixtures of GMR 134 with and without TFA (50 μg/cm²) against OR and PR strains of Ae. Aegypti are provided in FIG. 21. Provided in Table 6 are one hr repellency EC₅₀ values in g/cm² for some pyrethroid acids. EC₅₀ values were calculated from non-linear regression to a four parameter logistic equation (Prism, GraphPad Software, San Diego, Calif.) of the curves shown in FIG. 21. RR=resistance ratio. Worthwhile noting are the low levels of resistance to repellency by GMR 134 itself, as well as synergistic mixtures with TFA (50 μg/cm²) in the insecticide-resistant PR strain of Ae. aegypti. These results demonstrate low cross resistance of synergistic mixtures.

TABLE 6

| Chemical | GMR 134 | +TFA |
|---|---|---|
| 1 h EC₅₀-OR | 6.5 | 0.42 |
| 1 h EC₅₀-PR | 16 | 0.95 |
| RR (PR EC₅₀/OR EC₅₀) | 2.4 | 2.3 |

### Example 13: Synergistic Compositions Comprising TFA and Benzaldehyde

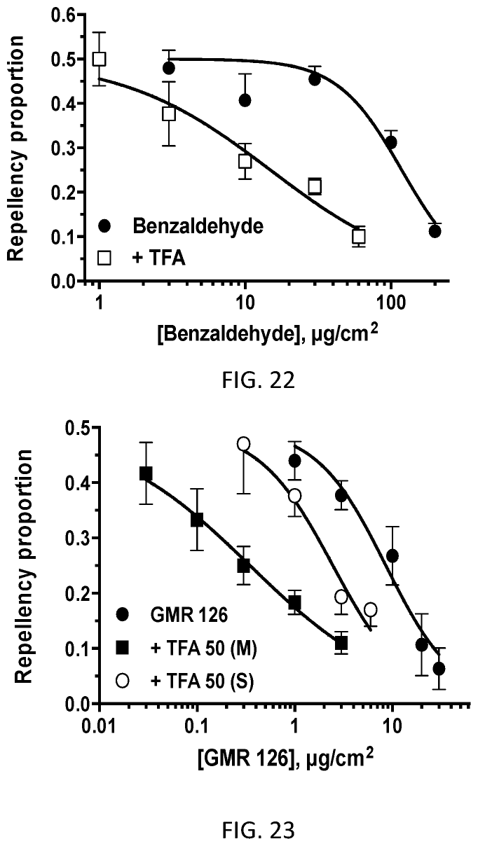

Provided in FIG. 22 are response curves demonstrating un-substituted benzaldehyde repellency on Orlando mosquitoes. Alone, un-substituted benzaldehyde exhibited EC₅₀=118 μg/cm². When combined with TFA (50 μg/cm²), the EC₅₀ was 15 μg/cm², demonstrating an 8-fold increase.

FIG. 23 shows concentration-response curves for repellency of GMR 126 with TFA (50 μg/cm²) applied as a mixture (M) or separate (S) treated halves of the same filter. Compounds were tested against the insecticide-susceptible Orlando (OR) strain of Ae. aegypti. The EC₅₀ values with non-overlapping 95% confidence limits were statistically significant. Synergism was present, but reduced when applied separately. This data is summarized in Table 7.

TABLE 7

| GMR 126 | 1 h EC₅₀ | 95% Confidence Limits |
|---|---|---|
| GMR 126 | 8.6 | 6-12 |
| +TFA Mixed | 0.35 | 0.2-0.6 |
| +TFA Separate | 2.5 | 1.6-3.9 |

### Example 14: Investigation of Benzaldehyde Derivatives as Vapor Active Repellents and Toxicants

Table 8 shows the differential efficacy of benzaldehyde derivatives as both vapor active repellents and toxicants at screening concentrations. All data is reported as repellency ratios and percentage mortality at 24 hr. Fractions represent repellency as defined by Jiang et al. (Jiang S., Yang L, and Bloomquist J (2019). Med. Vet. Entomol.), where 0.5=0% repellency and 0=100% repellency. Numbers for 3-tert-butylbenzaldehyde and 3-chlorobenzaldehyde are % mortality, since they were rapidly lethal in 1 hr exposures.

TABLE 8

| | | Horizontal tube orientation | | | |
|---|---|---|---|---|---|
| | | | | | 24 hr |
| Treatment | Concentration | 15 min | 30 min | 1 hr | mort (%) |
| Control (acetone) | | 0.50 | 0.46 | 0.48 | 2.1 |
| m-toluamide | 100 μg/cm² | 0.19 | 0.31 | 0.06 | NA |
| | 10 μg/cm² | 0.50 | 0.50 | 0.38 | NA |
| benzyloxybenzaldehyde | 100 μg/cm² | 0.69 | 0.81 | 0.44 | NA |
| | 10 μg/cm² | 0.69 | 0.69 | 0.63 | NA |
| 3-tert-butylbenzaldehyde | 100 μg/cm² | 96.88 | 100.00 | 100.00 | 81.3 |
| | 10 μg/cm² | 0.31 | 0.25 | 0.31 | 6.3 |
| 3-formyl benzoic acid | 100 μg/cm² | 0.53 | 0.56 | 0.56 | 9.4 |
| | 10 μg/cm² | 0.50 | 0.47 | 0.47 | 6.3 |
| 3-hydroxybenzaldehyde | 100 μg/cm² | 0.41 | 0.41 | 0.56 | 15.6 |
| | 10 μg/cm² | 0.56 | 0.53 | 0.56 | 21.9 |
| isophthalaldehdye | 100 μg/cm² | 0.03 | 0.09 | 0.06 | 87.5 |
| | 10 μg/cm² | 0.41 | 0.41 | 0.41 | 40.6 |
| 3-chlorobenzaldehyde | 100 μg/cm² | 50.00 | 75.00 | 100.00 | 100.0 |
| | 10 μg/cm² | 0.28 | 0.34 | 0.41 | 31.3 |
| α, α, α-trifluoromethyltolualdehyde | 100 μg/cm² | 0.38 | 0.50 | 0.63 | 0.0 |
| | 10 μg/cm² | 0.38 | 0.44 | 0.31 | 0.0 |
| 3-vinylbenzaldehyde | 100 μg/cm² | 0.19 | 0.25 | 0.19 | 6.3 |
| | 10 μg/cm² | 0.50 | 0.50 | 0.31 | 100.0 |
| biphenyl-3-carboxaldehyde | 100 μg/cm² | 0.44 | 0.31 | 0.19 | 6.3 |
| | 10 μg/cm² | 0.44 | 0.38 | 0.38 | 50.0 |
| trifluoromethoxybenzaldehyde | 100 μg/cm² | 0.31 | 0.31 | 0.50 | 0.0 |
| | 10 μg/cm² | 0.38 | 0.38 | 0.44 | 6.3 |

Example 15: Synergism of Synthetic Pyrethroid Spatial Repellency and Vapor Toxicity with Essential Oils on Susceptible Orlando Ae. aegypti

Table 9 shows Synergism ratios of 24 hr vapor toxicity by plant essential oils (EO) at two concentrations; the repellency EC₂₀ of each oil and at 100 μg/cm², on Aedes aegypti adult female mosquitoes. Synergism ratio (SR)=LC₅₀ pyrethroid/LC₅₀ pyrethroid+EO.

TABLE 9

| | Synergism Ratios | | | | | |
|---|---|---|---|---|---|---|
| | empenthrin | | metofluthrin | | transfluthrin | |
| | @EC₂₀ | @100 | @EC₂₀ | @100 | @EC₂₀ | 100 |
| | oil | μg/cm2 | oil | μg/cm2 | oil | μg/cm2 |
| +citronella | 2.29 | 2.83 | 6.33 | 30.40 | NA | 18.00 |
| +Amyris | 6.57 | 18.88 | 16.52 | 40.00 | 12.00 | 10.91 |
| +geranium | 1.26 | 15.10 | 2.81 | 28.15 | 5.14 | 36.00 |
| +CWT | 7.68 | 36.45 | 44.71 | 38.00 | 12.00 | 15.00 |
| (Cedarwood | | | | | | |
| Texas oil) | | | | | | |
| +CWV | 10.07 | 75.50 | 28.15 | 380.00 | 15.00 | 6.32 |
| (Cedarwood | | | | | | |
| Virginia oil) | | | | | | |
| +fir needle | 2.04 | 4.19 | 1.73 | 1.17 | 3.00 | 0.69 |
| +galbanum | — | — | — | 5.85 | — | — |
| +dill seed | — | — | — | 5.07 | — | — |
| +Canadian balsam | — | — | — | 15.20 | — | — |
| +cypress oil | — | — | — | 2.53 | — | — |
| +ginger root | — | — | — | 10.86 | — | — |

### Example 16: Additional Investigations of Plant Essential Oils in Combination with Metofluthrin

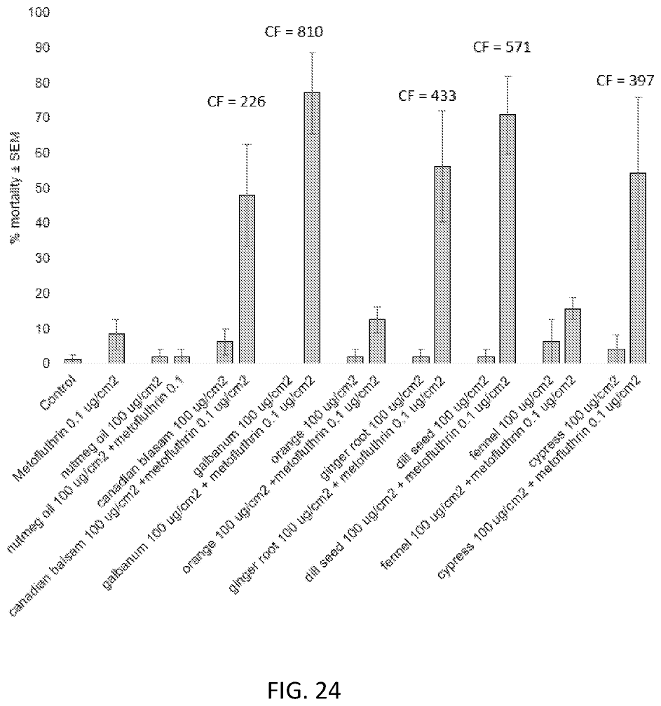

The mortality of plant essential oils applied at various concentrations alone or in combination with 0.1 μg/cm² in vapor toxicity assays on insecticide-susceptible Aedes aegypti (Orlando strain) females was investigated. The results are provided in FIG. 24. The most significant synergists of metofluthrin activity were Canadian balsam, galbanum, ginger root, dill seed, and cypress oils. Co-toxicity factors (CF) can indicate a measure of synergism, as described by Mansour et al. 1966, Toxicological studies on the Egyptian cotton Leafworm, Prodenia litura. VI. Potentiation and antagonism of organophosphorus and carbamate insecticides. J. Econ Entomol 59(2): 307-311. CF values greater than 20 are considered synergistic.

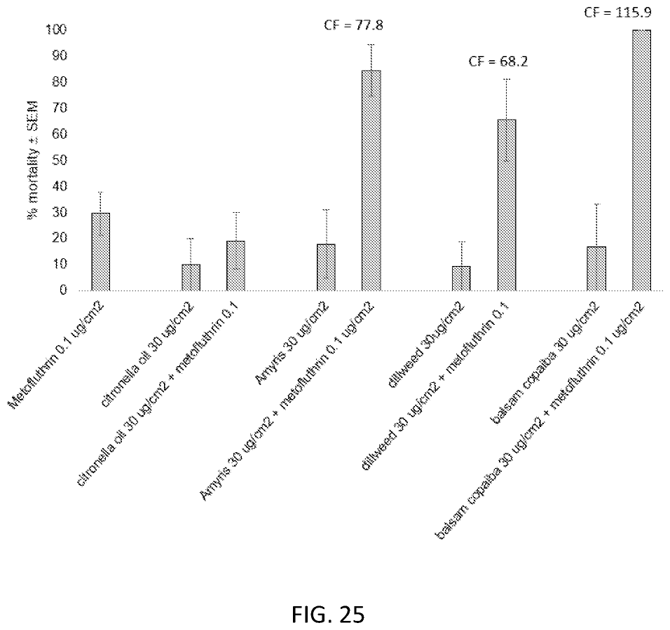

Next, the mortality of plant essential oils applied at various concentrations alone or in combination with 0.1 μg/cm² in vapor toxicity assays on insecticide-susceptible Anopheles gambiae (G3 strain) females was investigated. The results are provided in FIG. 25. The most significant synergists of metofluthrin activity of those screened were Amyris, dillweed, and balsam copaiba oil. In accordance with the teachings of Mansour et al. 1966, CF values greater than 20 are considered synergistic.

### Example 17: Further Investigations of Synergism of Pyrethroid Spatial Repellency and Vapor Toxicity with Citronella Oil on Pyrethroid-Resistant Puerto Rico Ae. aegypti

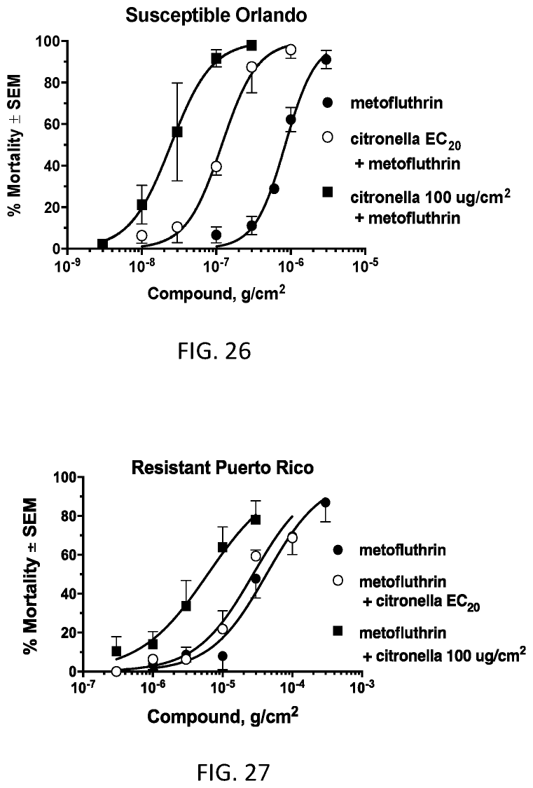

The vapor toxicity of metofluthrin alone and in combination with citronella oil (CO) on susceptible Orlando and pyrethroid-resistant Puerto Rico strains of Ae. aegypti was investigated. The results are provided in Table 10 and in FIGS. 26 and 27. Significant synergism was observed for combined mixtures of metofluthrin and citronella oil. Synergism ratio (SR)=LC50 metofluthrin/LC50 metofluthrin+citronella. Moreover, resistance to metofluthrin (RR=50.7) was significantly reduced by co-applying metofluthrin in combination with citronella oil (RR=7.3). Resistance ratio (RR)=LC₅₀ metofluthrin+CO on Puerto Rico/LC₅₀ metofluthrin on Orlando.

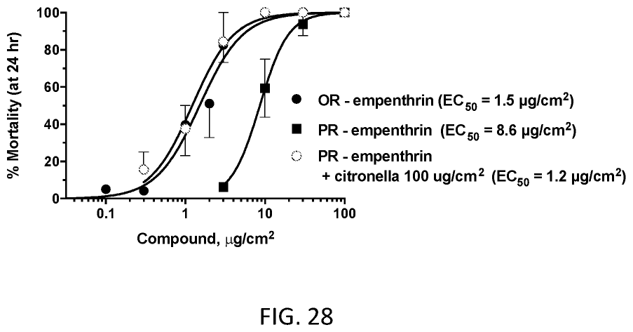

Next, the vapor toxicity of empenthrin applied to two strains of Aedes aegypti, the pyrethroid-susceptible Orlando and pyrethroid-resistant Puerto Rico strain, was investigated. The results are shown in FIG. 28. Empenthrin was applied alone and in combination with citronella oil (100 μg/cm², a nontoxic exposure). The RR for empenthrin alone was 5.7-fold. Citronella oil reversed resistance to empenthrin in the Puerto Rico strain, giving toxicity equivalent to empenthrin alone in the Orlando strain.

### Example 18: Preparation of nPentyl (1R,3S)-3-(2,2-dichlorovinyl)-2,2-dimethylcyclopropane-1-carboxylate (GMR 120)

The title compound was prepared using General Method B from 1-pentanol (0.043 mL, 0.397 mmol), 1R-trans-permethrinic acid (0.12 g, 0.562 mmol), N,N-Dicyclohexylcarbodiimide (DCC) (0.102 g, 0.49 mmol), 4-Dimethylaminopyridine (DMAP) (8.6 mg, 0.07 mmol) and dichloromethane (DCM) (1.5 mL). Automated flash chromatography of the crude oil yielded the product as a clear oil (110.5 mg, 99% yield). ¹H NMR (500 MHz, Chloroform-d) δ 5.62 (d, J=8.4 Hz, 1H), 4.08 (td, J=6.8, 3.9 Hz, 2H), 2.23 (dd, J=8.4, 5.3 Hz, 1H), 1.68-1.59 (m, 3H), 1.38-1.32 (m, 4H), 1.29 (s, 3H), 1.19 (s, 3H), 0.91 (t, J=7.1 Hz, 3H). ¹³C NMR (126 MHz, Chloroform-d) δ 171.10, 127.12, 121.84, 64.80, 34.85, 32.69, 28.68, 28.39, 28.06, 22.59, 22.31, 20.05, 13.94.

### Example 19: Preparation Cyclopentyl (1R,3S)-3-(2,2-dichlorovinyl)-2,2-dimethylcyclopropane-1-carboxylate (GMR 124)

The title compound was prepared using General Method B from cyclopentanol (0.0426 mL, 0.469 mmol), 1R-trans-permethrinic acid (0.12 g, 0.562 mmol), DCC (0.102 g, 0.49 mmol), DMAP (8.6 mg, 0.07 mmol) and DCM (1.5 mL). Automated flash chromatography of the crude oil yielded the product as a clear oil (111.5 mg, 86%). 1H NMR (500 MHz, Chloroform-d) δ 5.61 (d, J=8.4 Hz, 1H), 5.17 (tt, J=5.9, 2.9 Hz, 1H), 2.21 (dd, J=8.4, 5.4 Hz, 1H), 1.87 (dddd, J=13.0, 8.0, 3.7, 1.5 Hz, 2H), 1.79-1.54 (m, 7H), 1.29 (s, 3H), 1.19 (s, 3H). 13C NMR (126 MHz, Chloroform-d) δ 170.84, 127.24, 121.75, 77.36, 35.13, 32.86, 32.69, 32.57, 28.63, 23.84-23.69 (m), 22.65, 20.05.

### Example 20: Preparation of Cyclobutyl (1R,3S)-3-(2,2-dichlorovinyl)-2,2-dimethylcyclopropane-1-carboxylate (GMR 125)

The title compound was prepared using General Method B from cyclobutanol (0.0367 mL, 0.469 mmol), 1R-trans-permethrinic acid (0.12 g, 0.562 mmol), DCC (0.102 g, 0.49 mmol), DMAP (8.6 mg, 0.07 mmol) and DCM (1.5 mL). Automated flash chromatography of the crude oil yielded the product as a clear oil (106.15 mg, 86% yield). ¹H NMR (500 MHz, Chloroform-d) δ 5.61 (d, J=8.4 Hz, 1H), 4.99 (p, J=7.6 Hz, 1H), 2.36 (dtdd, J=11.3, 5.3, 2.7, 1.6 Hz, 2H), 2.22 (dd, J=8.4, 5.4 Hz, 1H), 2.14-2.03 (m, 2H), 1.81 (tddt, J=11.6, 9.0, 2.7, 1.3 Hz, 1H), 1.68-1.59 (m, 1H), 1.58 (d, J=5.3 Hz, 1H), 1.29 (s, 3H), 1.20 (s, 3H). ¹³C NMR (126 MHz, Chloroform-d) δ 170.40, 127.12, 121.86, 69.02, 34.77, 32.66, 30.45, 30.44, 28.79, 22.61, 20.00, 13.56.

### Example 21: Preparation of Cyclopropyl (1R,3S)-3-(2,2-dichlorovinyl)-2,2-dimethylcyclopropane-1-carboxylate (GMR 126)

The title compound was prepared using General Method B from cyclopropanol (0.0297 mL, 0.469 mmol), 1R-trans-permethrinic acid (0.12 g, 0.562 mmol), DCC (0.102 g, 0.49 mmol), DMAP (8.6 mg, 0.07 mmol) and DCM (1.5 mL). Automated flash chromatography of the crude oil yielded the product as a clear oil (94.9 mg, 81% yield). ¹H NMR (500 MHz, Chloroform-d) δ 5.60 (d, J=8.3 Hz, 1H), 4.20-4.04 (m, 1H), 2.23 (dd, J=8.4, 5.3 Hz, 1H), 1.55 (d, J=5.3 Hz, 1H), 1.29 (s, 3H), 1.18 (s, 3H), 0.76-0.64 (m, 4H). ¹³C NMR (126 MHz, Chloroform-d) δ 171.88, 126.94, 122.03, 49.05, 34.60, 32.86, 29.01, 22.56, 19.98, 5.16, 5.12.

### Example 22: Preparation of (1R,3S)-3-(2,2-dichlorovinyl)-2,2-dimethyl-N-(2,2,2-trifluoroethyl)cyclopropane-1-carboxamide (GMR 127)

Prepared from the general procedure above (Method B) from 2,2,2-trifluoroethan-1-amine (0.0368 mL, 0.469 mmol), 1R-trans-permethrinic acid (0.12 g, 0.562 mmol), DCC (0.102 g, 0.49 mmol), DMAP (8.6 mg, 0.07 mmol) and DCM (1.5 mL). Automated flash chromatography of the crude oil yielded the product as a clear oil (112.78 mg, 83% yield). ¹H NMR (500 MHz, Chloroform-d) δ 6.65 (t, J=6.5 Hz, 1H), 5.62 (d, J=8.2 Hz, 1H), 4.02-3.79 (m, 2H), 2.28 (dd, J=8.2, 5.2 Hz, 1H), 1.50 (d, J=5.2 Hz, 1H), 1.25 (s, 3H), 1.18 (s, 3H). ¹³C NMR (126 MHz, Chloroform-d) δ 170.37, 127.07, 122.97 (q, J=278.4 Hz), 121.99, 40.71 (q, J=34.7 Hz), 36.39, 31.46, 30.89, 22.57, 19.72.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A compound of Formula I 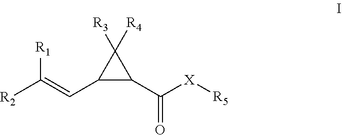
wherein R₁ and R₂ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R₁ and R₂ are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R₃ and R₄ are each independently selected from hydrogen, haloalkyl, alkenyl, alkynyl, and C₁-C₆ alkyl;
R₅ is selected from the group consisting of linear or branched C₅-C₁₂ alkyl, haloalkyl, alkenyl, alkynyl, C₃-C₅ cycloalkyl, C₃-C₁₂ cycloalkylalkyl, C₆-C₁₂ heteroaryl, and C₃-C₁₂ heterocycloalkyl; and
X is selected from the group consisting of O, NH, and S.

2. The compound of claim 1, wherein R₃ and R₄ are each C₁-C₃ alkyl.

3. The compound of claim 2, wherein R₃ and R₄ are each methyl.

4. The compound of claim 1, wherein R₁ and R₂ are each halo.

5. The compound of claim 4, wherein R₁ and R₂ are each independently selected from the group consisting of chloro, bromo, iodo, and fluoro.

6. The compound of claim 5, wherein R₁ and R₂ are each independently chloro.

7. The compound of claim 1, wherein X is O.

8. The compound of claim 1, wherein X in NH.

9. The compound of claim 1, wherein R₅ is selected from the group consisting of linear C₅-C₁₂ alkyl, haloalkyl, and C₃-C₅ cycloalkyl.

10. The compound of claim 9, wherein R₅ is selected from the group consisting of pentyl, trifluoroethyl, cyclobutyl, cyclopentyl, and cyclopropyl.

11. The compound of claim 1, wherein said compound is selected from the group consisting of:

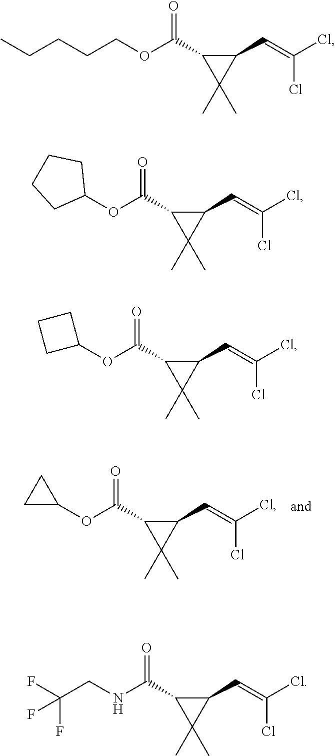 [SMILES: CCCCCOC(=O)[C@@H]1[C@@H](C=C(Cl)Cl)C1(C)C ; CC1(C)[C@H](C=C(Cl)Cl)[C@H]1C(=O)OC1CCCC1 ; CC1(C)[C@H](C=C(Cl)Cl)[C@H]1C(=O)OC1CCC1 ; CC1(C)[C@H](C=C(Cl)Cl)[C@H]1C(=O)OC1CC1 ; CC1(C)[C@H](C=C(Cl)Cl)[C@H]1C(=O)NCC(F)(F)F]

12. A composition comprising a compound of claim 1 and a carrier.

13. A composition comprising a compound of Formula II′ or a compound of Formula III′: 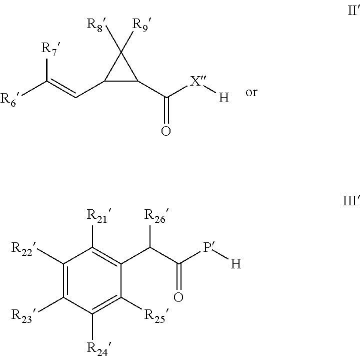
where R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl; and
a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, benzyl benzoate, ethyl hexanediol, diethyl phthalate, diethyl carbate, geraniol, nootkatone, citronellol, citronellal, citral, oil of lemon eucalyptus, cinnamaldehyde, and VUAA-1,
wherein the composition is synergistic, in admixture with a carrier.

14. The composition of claim 13, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.

15. The composition of claim 14, wherein said essential oil is citronella oil.

16. The composition of claim 13, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, empenthrin, and transfluthrin.

17. The composition of claim 13, wherein said composition comprises a compound of Formula II′, wherein X″ is O.

18. The composition of claim 17, wherein R_{8′} and R_{9′} are each methyl.

19. The composition of claim 13, wherein said compound of Formula II′ is

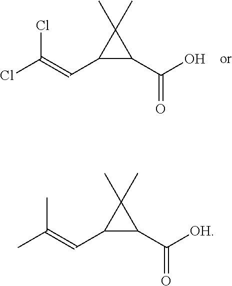

20. The composition of claim 13, comprising about 0.01-99.99% of 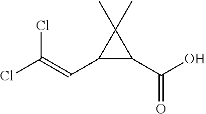
and about 99.99-0.01% transfluthrin.

21. The composition of claim 13, comprising about 0.01-99.99% 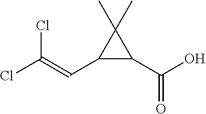
and about 99.99-0.01% citronella oil.

22. The composition of claim 13, wherein said composition comprises about 0.01-99.99%% of a compound of Formula II′ and about 99.99-0.01% citronella oil.

23. A composition comprising a compound of Formula IV, a compound of Formula V, or a compound of Formula VI: 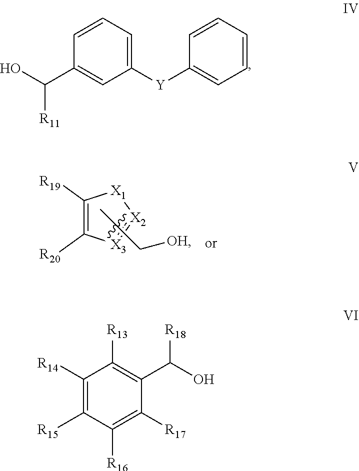
wherein Y is selected from the group consisting of O, NH, and S;
R₁₁ is selected from the group consisting of hydrogen, C₁-C₆ alkyl, alkenyl, alkynyl, CN, halo, and haloalkyl;
R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;
R₁₈ is selected from the group consisting of hydrogen, haloalkyl, and linear or branched C₁-C₆ alkyl; and
a compound of Formula II′ or a compound of Formula III′: 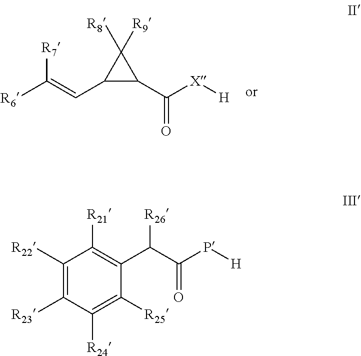
wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl,
wherein the composition is synergistic, in admixture with a carrier.

24. The composition of claim 23, wherein said composition comprises about 20-80% of a compound of Formula IV, a compound of Formula V, or a compound of Formula VI, and about 80-20% of a compound of Formula II′ or a compound of Formula III′.

25. The composition of claim 23, wherein said composition comprises about 40-60% of a compound of Formula IV, a compound of Formula V, or a compound of Formula VI, and about 60-40% of a compound of Formula II′ or a compound of Formula III′.

26. The composition of claim 23, wherein said composition comprises a compound of Formula VI and a compound of Formula II′.

27. The composition of claim 26, wherein said compound of Formula VI is 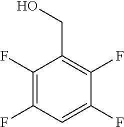 [SMILES: OCc1c(F)c(F)cc(F)c1F]
and said compound of Formula II′ is

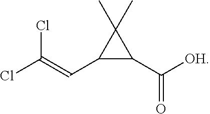

28. A composition comprising a compound of Formula VII 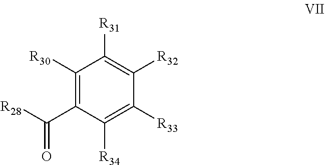
wherein R₂₈ is selected from the group consisting of hydrogen, haloalkyl, alkoxy, alkenyl, alkynyl, and C₁-C₆ linear or branched alkyl;
R₃₀, R₃₁, R₃₂, R₃₃, and R₃₄ are each independently selected from the group consisting of hydrogen, linear or branched C₁-C₆ alkyl, alkoxy, halo, cyano, haloalkyl, alkenyl, alkynyl, hydroxy, and 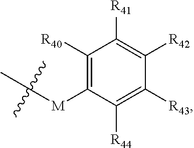
wherein M is selected from the group consisting of O, NH, and S;
R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy; and
a compound selected from the group consisting of an essential oil, a pyrethroid, methyl jasmonate, IR3535, 2-undecanone, picaridin, benzaldehyde, p-menthane-3,8-diol, alpha-terpineyl isovalerate, nootkatone, DEET, VUAA-1, a compound of Formula II′, a compound of Formula III′, a compound of Formula IV, a compound of Formula V, and a compound of Formula VI 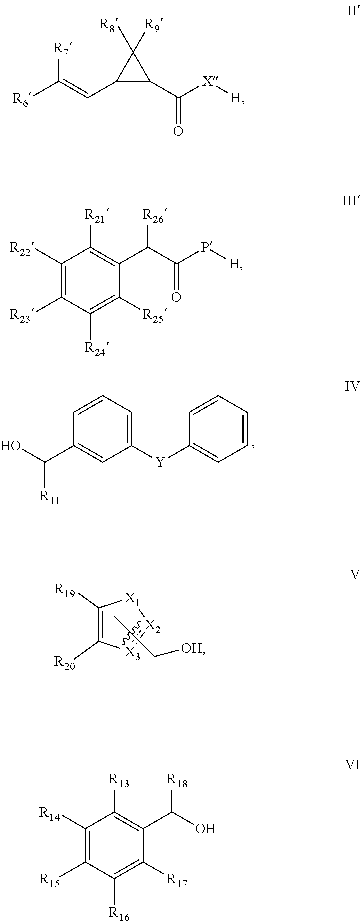
wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl;
Y is selected from the group consisting of O, NH, and S;
R₁₁ is selected from the group consisting of hydrogen, C₁-C₆ alkyl, alkenyl, alkynyl, CN, halo, and haloalkyl;
R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;
R₁₈ is selected from the group consisting of hydrogen, alkenyl, alkynyl, haloalkyl, and linear or branched C₁-C₆ alkyl,
wherein the composition is synergistic, in admixture with a carrier.

29. The composition of claim 28, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.

30. The composition of claim 29, wherein said essential oil is citronella oil.

31. The composition of claim 28, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, empenthrin, phenothrin, and transfluthrin.

32. The composition of claim 28, wherein said composition comprises about 0.01-99.99% of a compound of Formula VII and about 99.99-0.01% of a compound selected from citronella oil and a compound of Formula II′.

33. The composition of claim 28, wherein said composition comprises about 0.01-99.99% of a compound of Formula VII and about 99.99-0.01% of a compound selected from the group consisting of a compound of Formula IV, V, and VI.

34. The composition of claim 28, wherein R₃₀, R₃₁, R₃₂, and R₃₄ are each hydrogen.

35. The composition of claim 34, wherein R₃₃ is

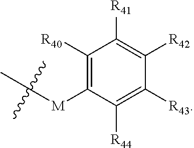

36. The compound of claim 35, wherein R₄₀, R₄₁, R₄₂, R₄₃, and R₄₄ are each hydrogen.

37. The composition of claim 36, wherein M is O.

38. The composition of claim 37, wherein R₂₉ is hydrogen.

39. The composition of claim 38, wherein said compound of Formula VII is

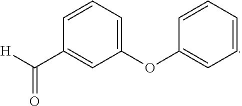 [SMILES: O=Cc1cccc(Oc2ccccc2)c1]

40. The composition of claim 28, wherein said composition comprises from 0.01-99.99% of a compound of Formula VII having the structure 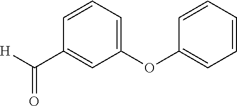 [SMILES: O=Cc1cccc(Oc2ccccc2)c1]
and from 99.99 to 0.01% of a compound of Formula II′ having the structure

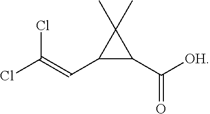

41. The composition of claim 28, wherein said composition comprises from 0.01-99.99% of a compound of Formula VII having the structure 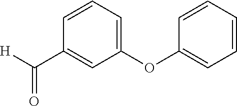 [SMILES: O=Cc1cccc(Oc2ccccc2)c1]
and from 99.99 to 0.01% of a compound of Formula II′ having the structure

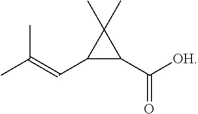

42. The composition of claim 28, wherein said composition comprises from 0.01-99.99% of a compound of Formula VII having the structure 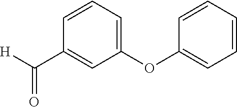 [SMILES: O=Cc1cccc(Oc2ccccc2)c1]
and from 99.99-0.1% of citronella oil.

43. The composition of claim 28, wherein said composition comprises from 0.01-99.99% of a compound of Formula VII having the structure 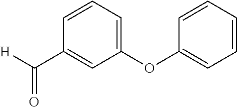 [SMILES: O=Cc1cccc(Oc2ccccc2)c1]
and from 99.99-0.01% of a compound of Formula VI having the structure

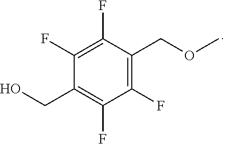

44. A composition comprising a compound of Formula I′, and a compound of Formula II′ or a compound of Formula III′ 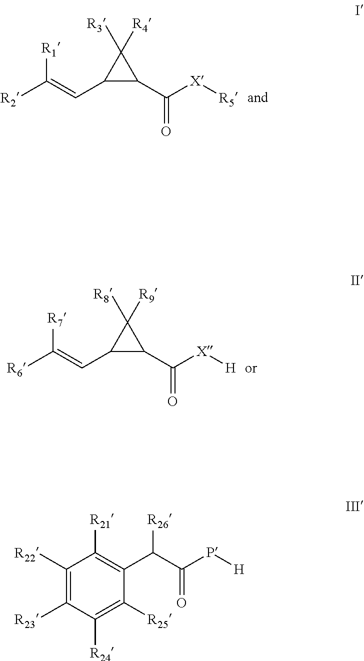
wherein R_{1′} and R_{2′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{1′} and R_{2′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{3′} and R_{4′} are each independently selected from hydrogen, haloalkyl, alkenyl, alkynyl, and C₁-C₆ alkyl;
R_{5′} is selected from the group consisting of linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
X′ is selected from the group consisting of O, NH, and S;
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{1′} and R_{2′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen, haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl, and
wherein the composition is synergistic, in admixture with a carrier.

45. The composition of claim 44, wherein said composition comprises from 0.01-99.99% of a compound of Formula I′ and from 99.99 to 0.01% of a compound of Formula II′, wherein said compound of Formula II′ is

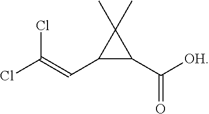

46. The composition of claim 44, wherein R₃, and R₄, are each methyl.

47. The composition of claim 44, wherein X′ is O.

48. The composition of claim 44, wherein R₅, is linear or branched C₁-C₆ alkyl.

49. The composition of claim 44, wherein R₅, is C₃-C₆ cycloalkyl.

50. A method of repelling an arthropod from an object or locus, comprising contacting said object or locus with a composition comprising a compound of Formula II′ or a compound of Formula III′: 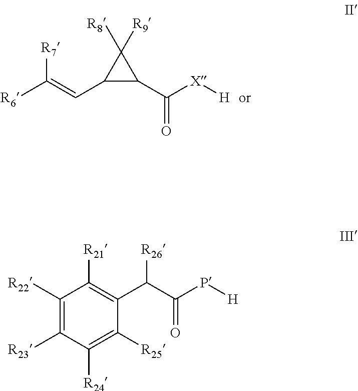
wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen, haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl; and
a compound selected from the group consisting of an essential oil, a pyrethroid, methyl jasmonate, IR3535, 2-undecanone, picaridin, benzaldehyde, p-menthane-3,8-diol, alpha-terpineyl isovalerate, nootkatone, DEET, benzyl benzoate, ethyl hexanediol, diethyl phthalate, diethyl carbate, geraniol, citronellol, citronellal, citral, oil of lemon eucalyptus, cinnamaldehyde, and VUAA-1,
wherein the composition is synergistic, in admixture with a carrier.

51. The method of claim 50, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.

52. The method of claim 51, wherein said essential oil is citronella oil.

53. The method of claim 50, wherein said pyrethroid is selected from the group consisting of permethrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, empenthrin, and transfluthrin.

54. The method of claim 50, wherein the composition contains about 0.0001 to 100000 parts per million of a compound of Formula II′ or a compound of Formula III′.

55. The method of claim 50, wherein the composition is a solution, dust, granular formulation or emulsion.

56. The method of claim 50, wherein the composition is a liquid solution or emulsion.

57. The method of claim 56, wherein the composition contains about 0.0001 to 100000 parts per million of a compound of Formula II′ or a compound of Formula III′.

58. The method of claim 50, wherein said arthropod is a fly, spider, butterfly, crab, mosquito, centipede, tick, millipede, or scorpion.

59. The method of claim 58, wherein said arthropod is a mosquito.

60. The method of claim 50, where the object or locus is an area, an environment, or the skin of an animal.

61. The method of claim 50, wherein said composition comprises a compound of Formula II′.

62. The method of claim 61, wherein X″ is O.

63. The method of claim 62, wherein R_{8′} and R_{9′} are each C₁-C₆ alkyl.

64. The method of claim 63, wherein R_{8′} and R_{9′} are each methyl.

65. The method of claim 64, wherein R_{7′} and R_{6′} are each independently halogen or C₁-C₆ alkyl.

66. The method of claim 65, wherein said compound of Formula II′ is

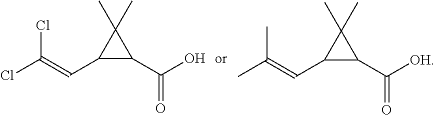

67. The method of claim 50, wherein said composition comprises 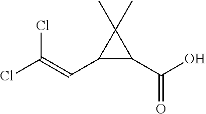
and transfluthrin.

68. The method of claim 50, wherein said composition comprises 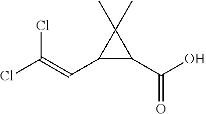
and citronella oil.

69. A method of repelling an arthropod from an object or locus, comprising contacting said object or locus with a composition comprising a compound of Formula IV, a compound of Formula V, or a compound of Formula VI 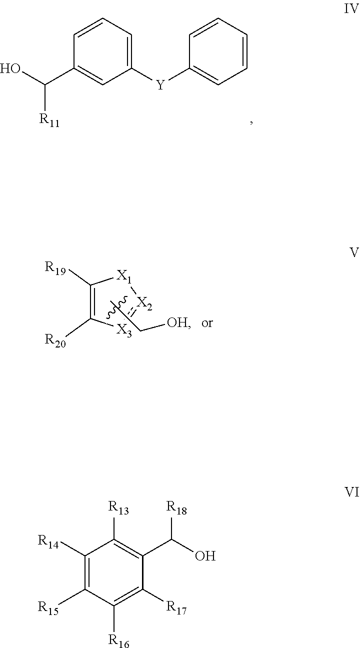
wherein Y is selected from the group consisting of O, NH, and S;
R₁₁ is selected from the group consisting of hydrogen, C₁-C₆ alkyl, alkenyl, alkynyl, CN, halo, and haloalkyl;
R₁₉ and R₂₀ are each independently selected from the group consisting of hydrogen, C₁-C₆ alkyl, haloalkyl, alkenyl, alkynyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, halo, haloalkyl, alkenyl, alkynyl, C₁-C₆ alkyl, and C₁-C₃ alkyl-alkoxy;
R₁₈ is selected from the group consisting of hydrogen, haloalkyl, alkenyl, alkynyl, and linear or branched C₁-C₆ alkyl; and
a compound of Formula II′ or a compound of Formula III′ 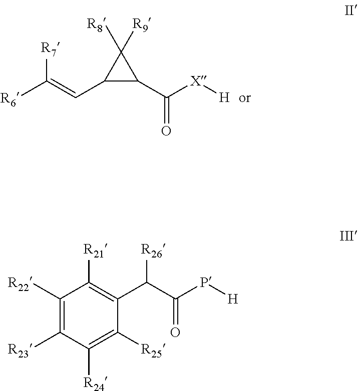
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen, haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl,
wherein the composition is synergistic, in admixture with a carrier.

70. The method of claim 69 wherein the composition contains about 0.0001 to 100000 parts per million of a compound of Formula VI.

71. The method of claim 69, wherein R₁₈ is hydrogen.

72. The method of claim 71, wherein R₁₃, R₁₄, R₁₆, and R₁₇ are each halo.

73. The method of claim 72, wherein R₁₃, R₁₄, R₁₆, and R₁₇ are each fluoro.

74. The method of claim 73, wherein R₁₆ is hydrogen or C₁-C₃ alkyl-alkoxy.

75. The method of claim 69, wherein said compound of Formula VI is

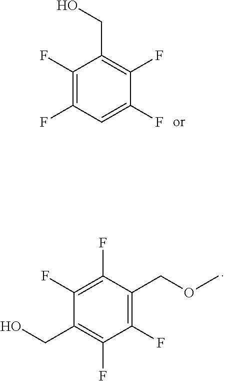

76. The method of claim 69, wherein said composition comprises a compound of Formula VI and a compound of Formula II′.

77. The method of claim 69, wherein X″ is O.

78. The method of claim 77, wherein R_{8′} and R_{9′} are each methyl.

79. The method of claim 78, wherein R_{7′} and R_{6′} are each independently halogen or C₁-C₆ alkyl.

80. The method of claim 69, wherein said compound of Formula VI is

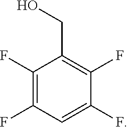 [SMILES: OCc1c(F)c(F)cc(F)c1F]

81. The method of claim 69, wherein said composition comprises a compound of Formula VI of 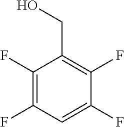 [SMILES: OCc1c(F)c(F)cc(F)c1F]
and a compound of Formula II′ of

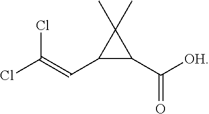

82. The method of claim 69, wherein the composition is a solution, dust, granular formulation, or emulsion.

83. The method of claim 69, wherein the composition is a liquid solution or emulsion.

84. The method of claim 83, wherein the composition contains about 0.0001 to 100000 parts per million of a compound of Formula IV, Formula V, or Formula VI.

85. The method of claim 69, wherein said arthropod is a fly, spider, butterfly, crab, mosquito, centipede, tick, millipede, or scorpion.

86. The method of claim 85, wherein said arthropod is a mosquito.

87. The method of claim 69, where the locus is an area, an environment, or the skin of an animal.

88. A method of repelling an arthropod from an object or locus, comprising contacting said object or locus with a composition comprising a compound of Formula VII 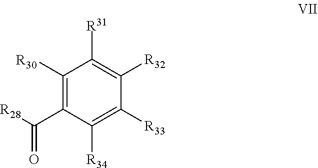
wherein R₂₈ is selected from the group consisting of hydrogen, haloalkyl, alkenyl, alkynyl, alkoxy, and C₁-C₆ linear or branched alkyl;
R₃₀, R₃₁, R₃₂, R₃₃, and R₃₄ are each independently selected from the group consisting of hydrogen, haloalkyl, alkenyl, alkynyl, linear or branched C₁-C₆ alkyl, alkoxy, halo, cyano, hydroxy, and 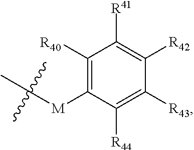
wherein M is selected from the group consisting of O, NH, and S;
R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy; and
a compound selected from the group consisting of an essential oil, a pyrethroid, methyl jasmonate, IR3535, 2-undecanone, picaridin, benzaldehyde, p-menthane-3,8-diol, alpha-terpineyl isovalerate, DEET, VUAA-1, nootkatone, a compound of Formula II′, a compound of Formula III′, a compound of Formula IV, a compound of Formula V, and a compound of Formula VI 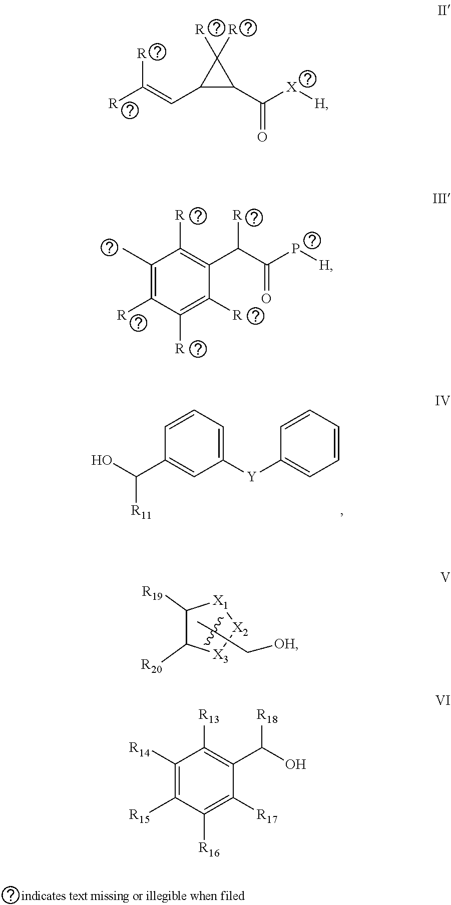
wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen, haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, haloalkyl, alkenyl, alkynyl, C₁-C₆ alkyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl;
Y is selected from the group consisting of O, NH, and S;
R₁₁ is selected from the group consisting of hydrogen, alkenyl, alkynyl, C₁-C₆ alkyl, CN, halo, and haloalkyl;
R₁₉ and R₂₀ are each independently selected from the group consisting of hydrogen, C₁-C₆ alkyl, cycloalkylalkyl, arylalkyl, haloalkyl, alkenyl, alkynyl, cycloalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, haloalkyl, alkenyl, alkynyl, halo, C₁-C₆ alkyl, and C₁-C₃ alkyl-alkoxy;
R₁₈ is selected from the group consisting of hydrogen, haloalkyl, alkenyl, alkynyl, and linear or branched C₁-C₆ alkyl,
wherein the composition is synergistic, in admixture with a carrier.

89. The method of claim 88 wherein the composition contains about 0.0001 to 100000 parts per million of a compound of Formula VII.

90. The method of claim 88, wherein R₃₀, R₃₁, R₃₂, and R₃₄ are each hydrogen.

91. The method of claim 90, wherein R₃₃ is

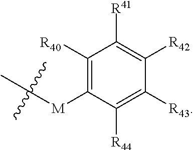

92. The method of claim 91, wherein R₄₀, R₄₁, R₄₂, R₄₃, and R₄₄ are each hydrogen.

93. The method of claim 92, wherein M is O.

94. The method of claim 93, wherein R₂₈ is hydrogen.

95. The method of claim 94, wherein said compound of Formula VII is

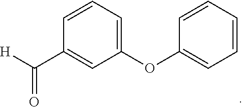 [SMILES: O=Cc1cccc(Oc2ccccc2)c1]

96. The method of claim 88, wherein said composition comprises 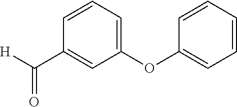 [SMILES: O=Cc1cccc(Oc2ccccc2)c1]
and citronella oil.

97. The method of claim 88, wherein said composition comprises 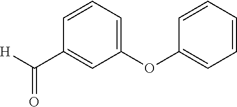 [SMILES: O=Cc1cccc(Oc2ccccc2)c1]
and a compound of Formula VI.

98. The method of claim 88, wherein said composition comprises

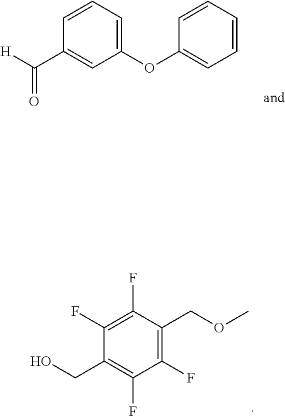

99. The method of claim 88, wherein said composition comprises 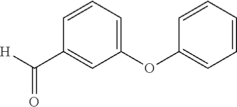 [SMILES: O=Cc1cccc(Oc2ccccc2)c1]
and a compound of Formula II′.

100. The method of claim 88, wherein said composition comprises

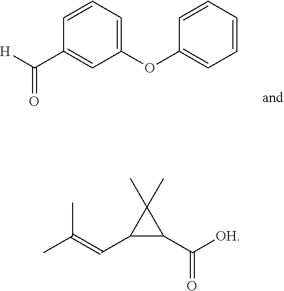

101. The method of claim 88, wherein said composition comprises

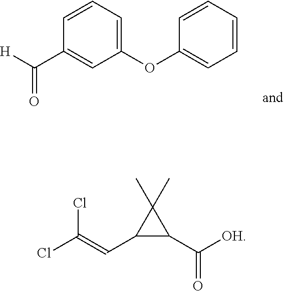

102. The method of claim 88, wherein the composition is a solution, dust, granular formulation or emulsion.

103. The method of claim 88, wherein the composition is a liquid solution or emulsion.

104. The method of claim 103, wherein the composition contains about 0.0001 to 100000 parts per million of a compound of Formula VII.

105. The method of claim 88, wherein said arthropod is a fly, spider, butterfly, crab, mosquito, centipede, tick, millipede, or scorpion.

106. The method of claim 105, wherein said arthropod is a mosquito.

107. The method of claim 88, where the object or locus is an area, an environment, or the skin of an animal.

108. A method of repelling an arthropod from an object or locus, comprising contacting said object or locus with a composition comprising a compound of Formula I′ 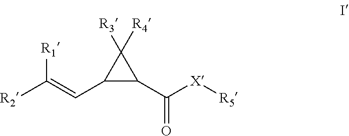
and a compound of Formula II′ or a compound of Formula III′ 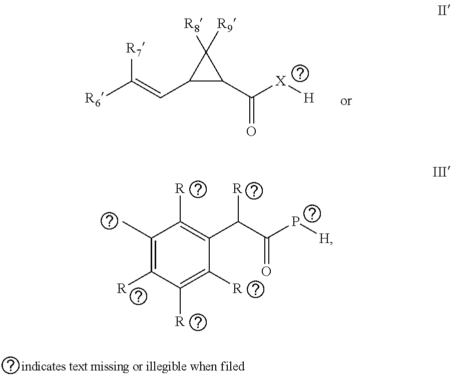
wherein R_{1′} and R_{2′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{3′} and R_{4′} are each independently selected from hydrogen, haloalkyl, alkenyl, alkynyl, and C₁-C₆ alkyl;
R_{5′} is selected from the group consisting of linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
X′ is selected from the group consisting of O, NH, and S;
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{8′} and R_{9′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R_{8′} and R_{9′} are each independently selected from hydrogen, haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl,
wherein the composition is synergistic, in admixture with a carrier.

109. The method of claim 108, wherein the composition contains about 0.0001 to 100000 parts per million of a compound of Formula I′.

110. The method of claim 108, wherein R₃, and R₄, are each methyl.

111. The method of claim 110, wherein X′ is O.

112. The method of claim 111, wherein R₅, is linear or branched C₁-C₆ alkyl.

113. The method of claim 108, wherein the composition is a solution, dust, granular formulation, or emulsion.

114. The method of claim 108, wherein the composition is a liquid solution or emulsion.

115. The method of claim 108, wherein the composition contains about 0.0001 to 100000 parts per million of the compound of Formula I′.

116. The method of claim 108, wherein the composition contains a compound of Formula I′ and a compound of Formula II′.

117. The method of claim 108, wherein X″ is O.

118. The method of claim 117, wherein R_{6′} and R_{7′} are each independently halo or C₁-C₆ alkyl.

119. The method of claim 118, wherein R_{8′} and R_{9′} are each methyl.

120. The method of claim 119, wherein the compound of Formula II″ is

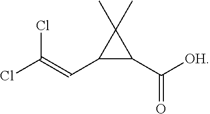

121. The method of claim 108 wherein the composition comprises a compound of Formula I′ and

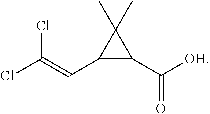

122. The method of claim 121, wherein the compound of Formula I′ is

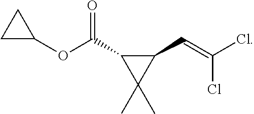

123. The method of claim 108, wherein said arthropod is a fly, spider, butterfly, crab, mosquito, centipede, tick, millipede, or scorpion.

124. The method of claim 123, wherein said arthropod is a mosquito.

125. The method of claim 108, where the object or locus is an area, an environment, or the skin of an animal.

126. A method for controlling one or more arthropods, comprising exposing said one or more arthropods to a synergistic composition comprising a compound of Formula II′ or a compound of Formula III′: 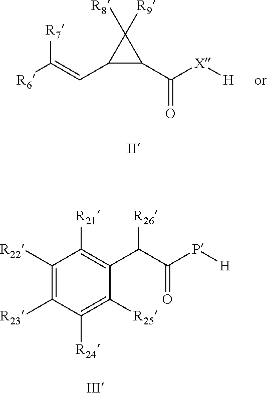
wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
R_{8′} and R_{9′} are each independently selected from hydrogen, haloalkyl, alkenyl, alkynyl, and C₁-C₆ alkyl;
X″ is selected from the group consisting of O, NH, and S;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S;
R_{26′} is selected from the group consisting of hydrogen, haloalkyl, alkenyl, alkynyl, and linear or branched C₁-C₆ alkyl; and
a pyrethroid,
wherein the synergistic composition produces, when the one or more arthropods are exposed to the synergistic composition, a combined toxicant effect greater than the sum of the separate toxicant effects from the compound of Formula II′ or Formula III′ and the pyrethroid, at comparable concentrations.

127. The method of claim 126, wherein said toxicant effect is the vapor toxicity of the synergistic composition.

128. The method of claim 126, wherein the synergistic composition contains about 0.0001 to 100000 parts per million of a compound of Formula II′ or Formula III′.

129. The method of claim 126, wherein said synergistic composition comprises a compound of Formula II′ and transfluthrin.

130. The method of claim 126, wherein X″ is O.

131. The method of claim 130, wherein R_{6′} and R_{7′} are halo or C₁-C₆ alkyl.

132. The method of claim 131, wherein R_{8′} and R_{9′} are each independently C₁-C₃ alkyl.

133. The method of claim 132, wherein R_{8′} and R_{9′} are each methyl.

134. The method of claim 133, wherein said compound of Formula II is

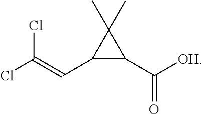

135. The method of claim 126, wherein said composition comprises 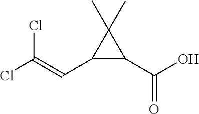
and transfluthrin.

136. The method of claim 126, wherein the synergistic composition is a solution, dust, granular formulation, or emulsion.

137. The method of claim 126, wherein the synergistic composition is a liquid solution or emulsion.

138. The method of claim 126, wherein the synergistic composition contains about 0.0001 to 100000 parts per million of a compound of Formula II′.

139. The method of claim 126, wherein said one or more arthropods are flies, spiders, butterflies, crabs, mosquitos, centipedes, ticks, millipedes, or scorpions.

140. The method of claim 139, wherein said one or more arthropods are mosquitos.

141. A method of repelling an arthropod from an object or locus, comprising contacting said object or locus with a composition comprising a carrier and a compound of Formula II, Formula III, Formula IV, Formula V, Formula VI, or Formula VII 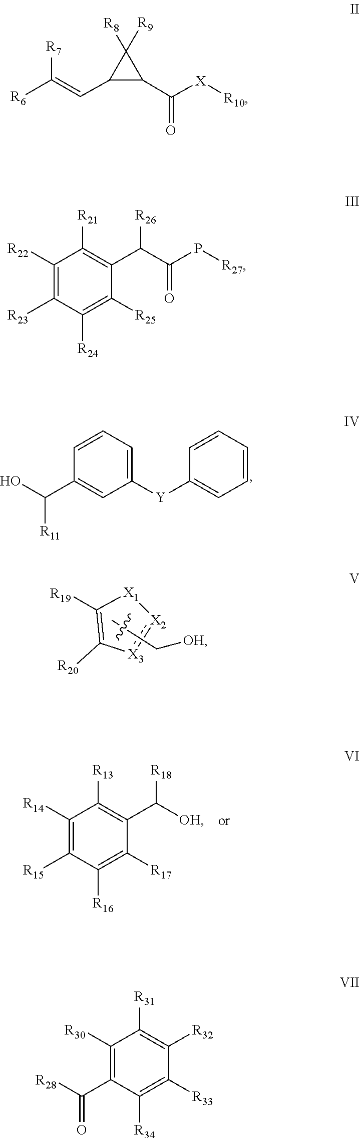
wherein R₆ and R₇ are each independently selected from the group consisting of hydrogen, halo, haloalkyl, C₆-C₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R₈ and R₉ are each independently selected from hydrogen, haloalkyl, alkenyl, alkynyl, and C₁-C₆ alkyl;
R₁₀ is selected from the group consisting of hydrogen, haloalkyl, alkenyl, alkynyl, linear or branched C₁-C₁₂ alkyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
X is selected from the group consisting of O, NH, and S;
R₂₁, R₂₂, R₂₃, R₂₄, and R₂₅ are each independently selected from the group consisting of hydrogen, alkoxy, halo, C₁-C₆ alkyl, haloalkyl, alkenyl, alkynyl, and C₁-C₃ alkyl-alkoxy;
P is selected from the group consisting of O, NH, and S;
R₂₆ is selected from the group consisting of hydrogen, haloalkyl, alkenyl, alkynyl, and linear or branched C₁-C₆ alkyl;
R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
R₁₁ is selected from the group consisting of hydrogen, C₁-C₆ alkyl, alkenyl, alkynyl, CN, halo, and haloalkyl;
R₁₉ and R₂₀ are each independently selected from the group consisting of hydrogen, haloalkyl, alkenyl, alkynyl, C₁-C₆ alkyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;
R₁₈ is selected from the group consisting of hydrogen, haloalkyl, alkenyl, alkynyl, and linear or branched C₁-C₆ alkyl;
R₂₈ is selected from the group consisting of hydrogen, haloalkyl, alkenyl, alkynyl, alkoxy, and C₁-C₆ linear or branched alkyl;
R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and 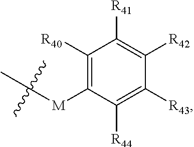
wherein M is selected from the group consisting of O, NH, and S; and
R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy.

142. The method of claim 141, wherein the composition is a solution, dust, granular formulation, or emulsion.

143. The method of claim 142, wherein the composition is a liquid solution or emulsion.

144. The method of claim 143, wherein said arthropod is a fly, spider, butterfly, crab, mosquito, centipede, tick, millipede, or scorpion.

145. The method of claim 144, wherein said arthropod is a mosquito.

146. The method of claim 145, wherein the mosquito is Aedes aegypti.

147. The method of claim 141, where the object or locus is an area, an environment, or the skin of an animal.

148. The method of claim 141, wherein said compound is of Formula II, wherein X is O and R₁₀ is hydrogen.

149. The method of claim 148, wherein R₈ and R₉ are each methyl.

150. The method of claim 149, wherein said compound is selected from the group consisting of

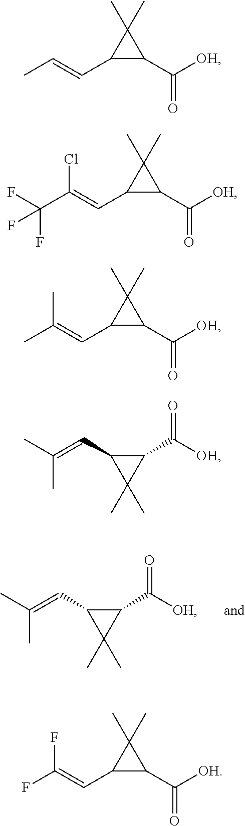

151. The method of claim 141, wherein said compound is of Formula III.

152. The method of claim 151, wherein P is O and R₂₇ is H.

153. The method of claim 152, wherein R₂₃ is halo and R₂₁, R₂₂, R₂₄, and R₂₅ are each hydrogen.

154. The method of claim 153, wherein R₂₃ is chloro.

155. The method of claim 154, wherein said compound is

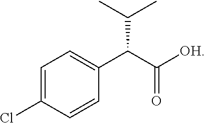

156. The method of claim 141, wherein said compound is of Formula VI.

157. The method of claim 156, wherein R₁₃, R₁₄, R₁₆, and R₁₇ are each halo.

158. The method of claim 157, wherein R₁₃, R₁₄, R₁₆, and R₁₇ are each fluoro.

159. The method of claim 158, wherein R₁₅ is hydrogen.

160. The method of claim 159, wherein R₁₈ is hydrogen.

161. The method of claim 160, wherein the compound of Formula VI is

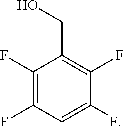 [SMILES: OCc1c(F)c(F)cc(F)c1F]

162. The method of claim 141, wherein the compound is of Formula IV.

163. The method of claim 162, wherein Y is O.

164. The method of claim 163, wherein R₁₁ is CN.

165. The method of claim 164, wherein the compound of Formula IV is

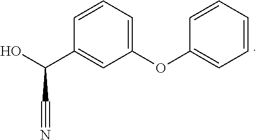 [SMILES: N#C[C@@H](O)c1cccc(Oc2ccccc2)c1]

166. The method of claim 141, wherein the compound is of Formula VII.

167. The method of claim 141, wherein R₃₀, R₃₁, R₃₂, and R₃₄ are each hydrogen.

168. The method of claim 167, wherein R₃₃ is

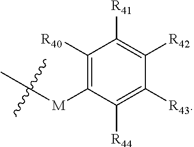

169. The method of claim 168, wherein R₄₀, R₄₁, R₄₂, R₄₃, and R₄₄ are each hydrogen.

170. The method of claim 169, wherein M is O.

171. The method of claim 170, wherein R₂₈ is H.

172. The method of claim 171, wherein the compound of Formula VII is

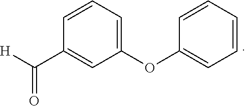 [SMILES: O=Cc1cccc(Oc2ccccc2)c1]

173. The method of claim 141, wherein the compound is of Formula II, wherein X is O and R₁₀ is selected from C₃-C₆ cycloalkyl and linear or branched C₁-C₆ alkyl.

174. A composition comprising an essential oil; and a second compound selected from the group consisting of a pyrethroid, a compound of Formula II, Formula III, Formula IV, Formula V, Formula VI, and Formula VII 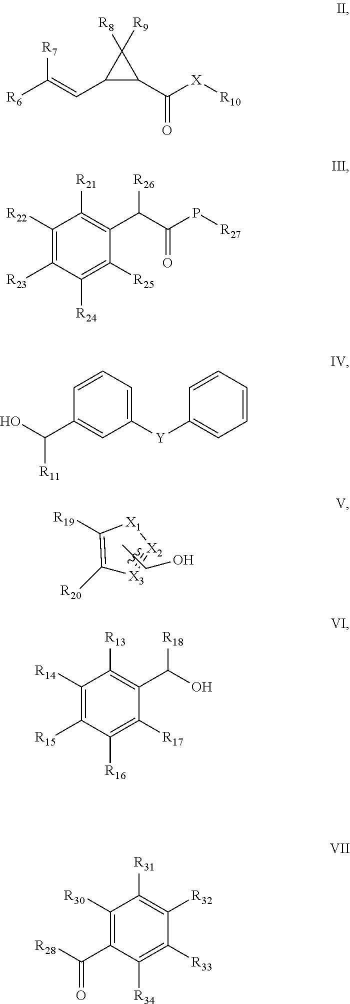
wherein R₆ and R₇ are each independently selected from the group consisting of hydrogen, halo, haloalkyl, C₆-C₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₂ heteroaryl, C₆-C₁₂ aryl, C₃-C₁₂ cycloalkyl, haloaryl, or C₃-C₁₂ heterocycloalkyl;
R₈ and R₉ are each independently selected from hydrogen haloalkyl, alkenyl, alkynyl, and C₁-C₆ alkyl;
R₁₀ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
X is selected from the group consisting of O, NH, and S;
R₂₁, R₂₂, R₂₃, R₂₄, and R₂₅ are each independently selected from the group consisting of hydrogen, alkoxy, halo, haloalkyl, alkenyl, alkynyl, C₁-C₆ alkyl, and C₁-C₃ alkyl-alkoxy;
P is selected from the group consisting of O, NH, and S;
R₂₆ is selected from the group consisting of hydrogen, haloalkyl, alkenyl, alkynyl, and linear or branched C₁-C₆ alkyl;
R₂₇ is selected from the group consisting of hydrogen, linear or branched C₁-C₁₂ alkyl, haloalkyl, alkenyl, alkynyl, C₃-C₁₂ cycloalkyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
R₁₁ is selected from the group consisting of hydrogen, C₁-C₆ alkyl, alkenyl, alkynyl, CN, halo, and haloalkyl;
R₁₉ and R₂₀ are each independently selected from the group consisting of hydrogen, C₁-C₆ alkyl, haloalkyl, alkenyl, alkynyl, cycloalkyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;
R₁₈ is selected from the group consisting of hydrogen, haloalkyl, alkenyl, alkynyl, and linear or branched C₁-C₆ alkyl;
R₂₈ is selected from the group consisting of hydrogen, haloalkyl, alkenyl, alkynyl, alkoxy, and C₁-C₆ linear or branched alkyl;
R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and 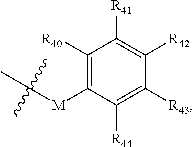
wherein M is selected from the group consisting of O, NH, and S;
R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy,
wherein the composition is synergistic, in admixture with a carrier.

175. The composition of claim 174, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.

176. The composition of claim 175, wherein said essential oil is citronella oil.

177. The composition of claim 174, wherein said second compound is a pyrethroid.

178. The composition of claim 177, wherein said pyrethroid is selected from the group consisting of permethrin, tetramethrin, metofluthrin, bifenthrin, kadethrin, allethrin, cyfluthrin, deltamethrin, empenthrin, cypermethrin, cyphenothrin, esfenvalerate, fenvalerate, flumethrin, tefluthrin, phenothrin, and transfluthrin.

179. The composition of claim 178, wherein said pyrethroid is metofluthrin.

180. The composition of claim 174, wherein said composition comprises a pyrethroid, wherein said pyrethroid is metofluthrin and said essential oil is selected from the group consisting of citronella oil, Canadian balsam, galbanum oil, ginger root oil, dill seed oil, and cypress oil.

181. The composition of claim 180, wherein said composition comprises metofluthrin having a concentration of about 0.1 μg/cm², and said essential oil having a concentration selected from the group consisting of 100 μg/cm², 30 μg/cm², and 20 μg/cm².

182. A method of repelling an arthropod from an object or locus, comprising contacting said object or locus with a composition comprising an essential oil, and a second compound selected from the group consisting of a pyrethroid, a compound of Formula II, Formula III, Formula IV, Formula V, Formula VI, and Formula VII 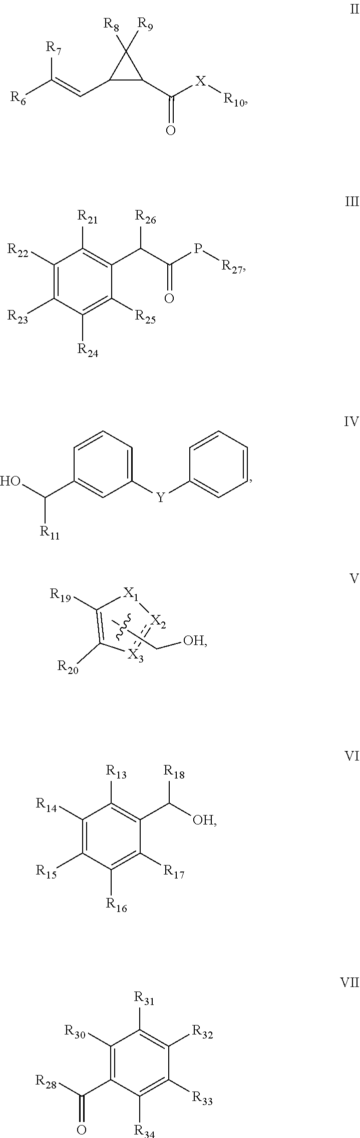
wherein R₆ and R₇ are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl;
R₈ and R₉ are each independently selected from hydrogen haloalkyl, alkenyl, alkynyl, and C₁-C₆ alkyl;
R₁₀ is selected from the group consisting of hydrogen, linear or branched C₁-C₁₂ alkyl, C₃-C₁₂ cycloalkyl, haloalkyl, alkenyl, alkynyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
X is selected from the group consisting of O, NH, and S;
R₂₁, R₂₂, R₂₃, R₂₄, and R₂₅ are each independently selected from the group consisting of hydrogen, alkoxy, halo, C₁-C₆ alkyl, haloalkyl, alkenyl, alkynyl, and C₁-C₃ alkyl-alkoxy;
P is selected from the group consisting of O, NH, and S;
R₂₆ is selected from the group consisting of hydrogen, haloalkyl, alkenyl, alkynyl, and linear or branched C₁-C₆ alkyl;
R₂₇ is selected from the group consisting of hydrogen, linear or branched C₁-C₁₂ alkyl, C₃-C₁₂ cycloalkyl, haloalkyl, alkenyl, alkynyl, C₃-C₁₂ cycloalkylalkyl, C₆-C₁₂ aryl, C₆-C₁₂ arylalkyl, C₆-C₁₂ heteroaryl, and C₃-C₁₂ heterocycloalkyl;
R₁₁ is selected from the group consisting of hydrogen, C₁-C₆ alkyl, alkenyl, alkynyl, CN, halo, and haloalkyl;
R₁₉ and R₂₀ are each independently selected from the group consisting of hydrogen, C₁-C₆ alkyl, cycloalkyl, haloalkyl, alkenyl, alkynyl, cycloalkylalkyl, arylalkyl, heterocycloalkyl, heteroarylalkyl, halo, and alkoxy, wherein said cycloalkyl, cycloalkylalkyl, arylalkyl, heterocycloalkyl, or heteroarylalkyl is optionally substituted with C₁-C₆ alkyl; or
where the substituents on R₁₉ and R₂₀, taken together with the carbon to which each is attached, form a C₅-C₇ cycloalkyl;
X₁, X₂, and X₃ are each independently selected from the group consisting of CH, CH₂, O, S, N, C═O, and NH; and
wherein at least one of X₁, X₂, and X₃ is substituted with —COH;
R₁₃, R₁₄, R₁₅, R₁₆, and R₁₇ are each independently selected from the group consisting of hydrogen, alkoxy, halo, C₁-C₆ alkyl, haloalkyl, alkenyl, alkynyl, and C₁-C₃ alkyl-alkoxy;
R₁₈ is selected from the group consisting of hydrogen, haloalkyl, alkenyl, alkynyl, and linear or branched C₁-C₆ alkyl;
R₂₈ is selected from the group consisting of hydrogen, haloalkyl, alkenyl, alkynyl, alkoxy, and C₁-C₆ linear or branched alkyl;
R₃₀, R₃₁, R₃₂, R₃₃, and R₃₄ are each independently selected from the group consisting of hydrogen, linear or branched C₁-C₆ alkyl, haloalkyl, alkenyl, alkynyl, alkoxy, halo, cyano, hydroxy, and 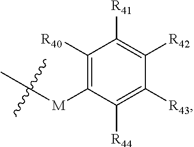
wherein M is selected from the group consisting of O, NH, and S;
R₄₀, R₄₁, R₄₂, R₄₃, and R₄₄ are each independently selected from the group consisting of hydrogen, linear or branched C₁-C₆ alkyl, haloalkyl, alkenyl, alkynyl, alkoxy, halo, cyano, hydroxy,
wherein the composition is synergistic, in admixture with a carrier.

183. The method of claim 182, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, Canadian balsam, nutmeg oil, fennel oil, dill weed oil, balsam copaiba, and tea tree oil.

184. The method of claim 183, wherein said essential oil is citronella oil.

185. The method of claim 182, wherein said second compound is a pyrethroid.

186. The method of claim 185, wherein said pyrethroid is selected from the group consisting of permethrin, empenthrin, tetramethrin, metofluthrin, bifenthrin, kadethrin, allethrin, cyfluthrin, deltamethrin, cypermethrin, cyphenothrin, esfenvalerate, fenvalerate, flumethrin, tefluthrin, phenothrin, and transfluthrin.

187. The method of claim 186, wherein said pyrethroid is metofluthrin.

188. The method of claim 182, wherein said composition comprises a pyrethroid, wherein said pyrethroid is metofluthrin, and said essential oil is selected from the group consisting of citronella oil, Canadian balsam, galbanum oil, ginger root oil, dill seed oil, and cypress oil.

189. The method of claim 188, wherein said composition comprises metofluthrin having a concentration of about 0.1 μg/cm², and said essential oil having a concentration selected from the group consisting of 100 μg/cm², 30 μg/cm², and 20 μg/cm².

190. The method of claim 182, wherein said essential oil is citronella oil and said second compound is empenthrin.

191. The method of claim 190, wherein said composition comprises empenthrin having a concentration of about 0.1 μg/cm², and said essential oil having a concentration of about 100 μg/cm².

192. The composition of claim 174, wherein said essential oil is citronella oil and said second compound is empenthrin.

193. The composition of claim 192, wherein said composition comprises empenthrin having a concentration of about 0.1 μg/cm², and said essential oil having a concentration of about 100 μg/cm².

194. A method for controlling one or more arthropods, comprising exposing said one or more arthropods to any single compound described herein, or a composition comprising any single compound and a carrier.

195. A method of repelling an arthropod from an object or locus, comprising contacting said object or locus with any single compound described herein, or a composition comprising any single compound and a carrier.

196. A method of repelling an arthropod from an object or locus, comprising contacting said object or locus with a compound in Table 8, or a composition comprising a compound in Table 8 and a carrier.

197. A method for controlling one or more arthropods, comprising exposing said one or more arthropods to a compound in Table 8, or a composition comprising a compound in Table 8 and a carrier.

198. A method for controlling one or more arthropods, comprising exposing said one or more arthropods to the composition of any one of claims 12-49 or 174-181.

199. The method of any one of claims 50, 69, 88, 108, 141, 182, 195, or 196, wherein said arthropod is repelled from an adjacent area to said object or locus, wherein said arthropod is not physically contacted with said object or locus.

200. A method of providing a composition of any one of claims 12-49 or 174-181 in an arthropod repellent medium from which a vaporized composition of any one of claims 13-49 or 174-181 can be dispersed.

201. The method of any one of claims 50, 69, 88, 108, 126, 194, 197, or 198, wherein the composition is formulated for use in a vaporizer, evaporator, fan, heat, candle, or wicked apparatus.

202. The method of claim 126, 194, 197, or 198, wherein the synergistic composition is a vapor.
